SEQUENCE LISTING (124 sequences)

SEQ ID NO: 1 - DNA, 28 bp, Arabidopsis thaliana
aagcttatggtgaggcctccttgttgtg

SEQ ID NO: 2 - DNA, 33 bp, Arabidopsis thaliana
tctagaccggatatgagcgagcattttttggtc

SEQ ID NO: 3 - DNA, 20 bp, Arabidopsis thaliana
gtgaaaaactcgccgaagac

SEQ ID NO: 4 - DNA, 20 bp, Arabidopsis thaliana
gcacactccttcccatcatc

SEQ ID NO: 5 - DNA, 21 bp, Arabidopsis thaliana
tcctagtaagcgcgagtcatc

SEQ ID NO: 6 - DNA, 20 bp, Arabidopsis thaliana
cgaacacttcaccggatcat

SEQ ID NO: 7 - DNA, 25 bp, Arabidopsis thaliana
tgctttcgccattaaatagcgacgg

SEQ ID NO: 8 - DNA, 23 bp, Arabidopsis thaliana
cgctgcggacatctacatttttg

SEQ ID NO: 9 - DNA, 22 bp, Arabidopsis thaliana
tcccggacatgaagccatttac

SEQ ID NO: 10 - DNA, 16 bp, Arabidopsis thaliana
  unsure (1)..(1): n stands for any base
  misc_feature (1)..(1): n is a, c, g, or t
  unsure (11)..(11): n stands for any base
  misc_feature (11)..(11): n is a, c, g, or t
ngtcgaswganawgaa

SEQ ID NO: 11 - protein, 323 aa, Arabidopsis thaliana
MVRPPCCDKGGVKKGPWTPEEDIILVTYIQEHGPGNWRAVPTNTGLLRCSKSCRLRWTNYLRPGIKRGNFTEHEEKMIVHLQALLGNRWAAIASYLPQRTDNDIKNYWNTHLKKKLNKVNQDSHQELDRSSLSSSPSSSSANSNSNISRGQWERRLQTDIHLAKKALSEALSPAVAPIITSTVTTTSSSAESRRSTSSASGFLRTQETSTTYASSTENIAKLLKGWVKNSPKTQNSADQIASTEVKEVIKSDDGKECAGAFQSFSEFDHSYQQAGVSPDHETKPDITGCCSNQSQWSLFEKWLFEDSGGQIGDILLDENTNFF

SEQ ID NO: 12 - DNA, 972 bp, Arabidopsis thaliana
atggtgaggcctccttgttgtgacaaaggaggagtgaagaaagggccatggactcctgaagaagatatcattttagtcacttacatccaagaacatggtcctggtaattggagagctgttcctaccaatactgggctgcttagatgcagcaagagttgtagacttagatggacaaactatttaaggccaggaatcaaaagaggcaatttcacagaacatgaagaaaagatgattgttcatctccaagccctcttaggaaatagatgggctgcaattgcgtcatatcttccacaaaggacagacaatgacattaagaactattggaacactcatttgaagaagaaactcaacaaagtcaatcaagattctcatcaagaacttgaccgttcctcgctctcatcttcaccatcgtcttcttctgctaattccaactcaaacatctcaagaggccaatgggaaaggcgacttcaaaccgatatccacttggcgaaaaaggctctctctgaggctttatctcctgccgttgcaccaatcattacatctacagtgacaacaacgtcttcctctgctgaatcaagacgctctacttcctcagctagcgggtttcttaggacgcaagaaacatctacaacttatgcctcaagcaccgaaaatatcgcgaaattgctcaaagggtgggtgaaaaactcgccgaagactcaaaactccgcggatcaaatcgcttctacagaggtaaaagaagtgatcaagagtgatgatgggaaggagtgtgcaggggcatttcagtcattttctgagtttgatcactcatatcaacaggctggtgtttcacctgatcatgagaccaaaccagacataactggatgctgcagtaaccaaagtcaatggtctttgtttgagaagtggttgtttgaggattctggtggacagattggtgatattctattggatgaaaacactaatttcttctga

SEQ ID NO: 13 - protein, 615 aa, Arabidopsis thaliana
MERRLMIPCFFWLILVLDLVLRVSGNAEGDALSALKNSLADPNKVLQSWDATLVTPCTWFHVTCNSDNSVTRVDLGNANLSGQLVMQLGQLPNLQYLELYSNNITGTIPEQLGNLTELVSLDLYLNNLSGPIPSTLGRLKKLRFLRLNNNSLSGEIPRSLTAVLTLQVLDLSNNPLTGDIPVNGSFSLFTPISFANTKLTPLPASPPPPISPTPPSPAGSNRITGAIAGGVAAGAALLFAVPAIALAWWRRKKPQDHFFDVPAEEDPEVHLGQLKRFSLRELQVASDNFSNKNILGRGGFGKVYKGRLADGTLVAVKRLKEERTQGGELQFQTEVEMISMAVHRNLLRLRGFCMTPTERLLVYPYMANGSVASCLRERPESQPPLDWPKRQRIALGSARGLAYLHDHCDPKIIHRDVKAANILLDEEFEAVVGDFGLAKLMDYKDTHVTTAVRGTIGHIAPEYLSTGKSSEKTDVFGYGVMLLELITGQRAFDLARLANDDDVMLLDWVKGLLKEKKLEALVDVDLQGNYKDEEVEQLIQVALLCTQSSPMERPKMSEVVRMLEGDGLAERWEEWQKEEMFRQDFNYPTHHPAVSGWIIGDSTSQIENEYPSGPR

SEQ ID NO: 14 - DNA, 1848 bp, Arabidopsis thaliana
atggaacgaagattaatgatcccttgcttcttttggttgattctcgttttggatttggttctcagagtctcgggcaacgccgaaggtgatgctctaagtgcactgaaaaacagtttagccgaccctaataaggtgcttcaaagttgggatgctactcttgttactccatgtacatggtttcatgttacttgcaatagcgacaatagtgttacacgtgttgaccttgggaatgcaaatctatctggacagctcgtaatgcaacttggtcagcttccaaacttgcagtacttggagctttatagcaataacattactgggacaatcccagaacagcttggaaatctgacggaattggtgagcttggatctttacttgaacaatttaagcgggcctattccatcaactctcggccgacttaagaaactccgtttcttgcgtcttaataacaatagcttatctggagaaattccaaggtctttgactgctgtcctgacgctacaagttctggatctctcaaacaatcctctcaccggagatattcctgttaatggttccttttcacttttcactccaatcagttttgccaacaccaagttgactccccttcctgcatctccaccgcctcctatctctcctacaccgccatcacctgcagggagtaatagaattactggagcgattgcgggaggagttgctgcaggtgctgcacttctatttgctgttccggccattgcactagcttggtggcgaaggaaaaagccgcaggaccacttctttgatgtaccagctgaagaggacccagaagttcatttaggacaactgaagaggttttcattgcgtgaactacaagttgcttcggataattttagcaacaagaacatattgggtagaggtggttttggtaaagtttataaaggacggttagctgatggtactttagtggccgttaaaaggctaaaagaggagcgcacccaaggtggcgaactgcagttccagacagaggttgagatgattagtatggcggttcacagaaacttgcttcggcttcgtggattttgcatgactccaaccgaaagattgcttgtttatccctacatggctaatggaagtgttgcctcctgtttaagagaacgtcccgagtcccagccaccacttgattggccaaagagacagcgtattgcgttgggatctgcaagagggcttgcgtatttacatgatcattgcgacccaaagattattcatcgagatgtgaaagctgcaaatattttgttggatgaagagtttgaagccgtggttggggattttggacttgcaaaactcatggactacaaagacacacatgtgacaaccgcagtgcgtgggacaattggtcatatagcccctgagtacctttccactggaaaatcatcagagaaaaccgatgtctttgggtatggagtcatgcttcttgagcttatcactggacaaagggcttttgatcttgctcgcctcgcgaatgatgatgatgtcatgttactagactgggtgaaagggttgttaaaagagaagaaattggaagcactagtagatgttgatcttcagggtaattacaaagacgaagaagtggagcagctaatccaagtggctttactctgcactcagagttcaccaatggaaagacccaaaatgtctgaagttgtaagaatgcttgaaggagatggtttagctgagagatgggaagagtggcaaaaggaggaaatgttcagacaagatttcaactacccaacccaccatccagccgtgtctggctggatcattggcgattccacttcccagatcgaaaacgaatacccctcgggtccaagataa

SEQ ID NO: 15 - protein, 1196 aa, Arabidopsis thaliana
MKTFSSFFLSVTTLFFFSFFSLSFQASPSQSLYREIHQLISFKDVLPDKNLLPDWSSNKNPCTFDGVTCRDDKVTSIDLSSKPLNVGFSAVSSSLLSLTGLESLFLSNSHINGSVSGFKCSASLTSLDLSRNSLSGPVTTLTSLGSCSGLKFLNVSSNTLDFPGKVSGGLKLNSLEVLDLSANSISGANVVGWVLSDGCGELKHLAISGNKISGDVDVSRCVNLEFLDVSSNNFSTGIPFLGDCSALQHLDISGNKLSGDFSRAISTCTELKLLNISSNQFVGPIPPLPLKSLQYLSLAENKFTGEIPDFLSGACDTLTGLDLSGNHFYGAVPPFFGSCSLLESLALSSNNFSGELPMDTLLKMRGLKVLDLSFNEFSGELPESLTNLSASLLTLDLSSNNFSGPILPNLCQNPKNTLQELYLQNNGFTGKIPPTLSNCSELVSLHLSFNYLSGTIPSSLGSLSKLRDLKLWLNMLEGEIPQELMYVKTLETLILDFNDLTGEIPSGLSNCTNLNWISLSNNRLTGEIPKWIGRLENLAILKLSNNSFSGNIPAELGDCRSLIWLDLNTNLFNGTIPAAMFKQSGKIAANFIAGKRYVYIKNDGMKKECHGAGNLLEFQGIRSEQLNRLSTRNPCNITSRVYGGHTSPTFDNNGSMMFLDMSYNMLSGYIPKEIGSMPYLFILNLGHNDISGSIPDEVGDLRGLNILDLSSNKLDGRIPQAMSALTMLTEIDLSNNNLSGPIPEMGQFETFPPAKFLNNPGLCGYPLPRCDPSNADGYAHHQRSHGRRPASLAGSVAMGLLFSFVCIFGLILVGREMRKRRRKKEAELEMYAEGHGNSGDRTANNTNWKLTGVKEALSINLAAFEKPLRKLTFADLLQATNGFHNDSLIGSGGFGDVYKAILKDGSAVAIKKLIHVSGQGDREFMAEMETIGKIKHRNLVPLLGYCKVGDERLLVYEFMKYGSLEDVLHDPKKAGVKLNWSTRRKIAIGSARGLAFLHHNCSPHIIHRDMKSSNVLLDENLEARVSDFGMARLMSAMDTHLSVSTLAGTPGYVPPEYYQSFRCSTKGDVYSYGVVLLELLTGKRPTDSPDFGDNNLVGWVKQHAKLRISDVFDPELMKEDPALEIELLQHLKVAVACLDDRAWRRPTMVQVMAMFKEIQAGSGIDSQSTIRSIEDGGFSTIEMVDMSIKEVPEGKL

SEQ ID NO: 16 - DNA, 3591 bp, Arabidopsis thaliana
atgaagactttttcaagcttctttctctctgtaacaactctcttcttcttctccttcttttctctttcatttcaagcttcaccatctcagtctttatacagagaaatccatcagcttataagcttcaaagacgttcttcctgacaagaatcttctcccagactggtcttccaacaaaaacccgtgtactttcgatggcgttacttgcagagacgacaaagttacttcgattgatctcagctccaagcctctcaacgtcggattcagtgccgtgtcctcgtctctcctgtctctcaccggattagagtctctgtttctctcaaactcacacatcaatggctccgtttctggcttcaagtgctctgcttctttaaccagcttggatctatctagaaactctctttcgggtcctgtaacgactctaacaagccttggttcttgctccggtctgaagtttcttaacgtctcttccaatacacttgattttcccgggaaagtttcaggtgggttgaagctaaacagcttggaagttctggatctttctgcgaattcaatctccggtgctaacgtcgttggttgggttctctccgatgggtgtggagagttgaaacatttagcgattagcggaaacaaaatcagtggagacgtcgatgtttctcgctgcgtgaatctcgagtttctcgatgtttcctccaacaatttctccactgggattcctttcctcggagattgctctgctctgcaacatcttgacatctccgggaacaaattatccggcgatttctcccgtgctatctctacttgcacagagctcaagttgttgaacatctctagtaaccaattcgtcggaccaatccctccgctaccgcttaaaagtctccaatacctctctctggccgagaacaaattcaccggcgagatccctgactttctctccggcgcgtgtgatacactcactggtctcgatctctctggaaatcatttctacggtgcggttcctccattcttcggttcatgttctcttctcgaatcactcgcgttgtcgagtaacaacttctctggcgagttaccgatggatacgttgttgaagatgagaggactcaaagtacttgatctgtctttcaacgagttttccggcgaattaccggaatctctgacgaatctatccgcttcgttgctaacgttagatctcagctccaacaatttctccggtccgattctcccaaatctctgccagaaccctaaaaacactctgcaggagctttaccttcagaacaatggcttcaccgggaagattccaccgactttaagcaactgttctgagctggtttcgcttcacttgagcttcaattacctctccgggacaatcccttcgagcttaggctctctatcgaagcttcgagatctgaaactatggctgaatatgttagaaggagagatccctcaggagctcatgtatgtcaagaccttagagactctgatcctcgacttcaacgatttaaccggtgaaatcccttccggtttaagtaactgtaccaatcttaactggatttctctgtcgaataaccggttaaccggtgagattccgaaatggattggccggttagagaatctcgctatcctcaagctaagcaacaattcattctccgggaacattccggctgagctcggcgactgcagaagcttaatctggcttgatctcaacaccaatctcttcaatggaacgattccggcggcgatgtttaaacaatccgggaaaatcgctgccaatttcatcgccggtaagaggtacgtttatatcaaaaacgatgggatgaagaaagagtgtcatggagctggtaatttacttgagtttcaaggaatcagatccgaacaattaaaccggctttcaacgaggaacccttgtaatatcactagcagagtctatggaggtcacacttcgccgacgtttgataacaatggttcgatgatgtttctggacatgtcttacaacatgttgtctggatacataccgaaggagattggttcgatgccttatctgtttattctcaatttgggtcataacgatatctctggttcgattcctgatgaggtaggtgatctaagaggtttaaacattcttgatctttcaagcaataagctcgatgggaggattcctcaggctatgtcagctcttactatgcttacggaaatcgatttgtcgaataataatttgtctggtccgattcctgagatgggtcagtttgagacttttccaccggctaagttcttgaacaatcctggtctctgtggttatcctcttccgcggtgtgatccttcaaatgcagacggttatgctcatcatcagagatctcatggaaggagaccagcgtcccttgctggtagtgtggcgatgggattgttgttctcttttgtgtgtatatttgggctgatccttgttggtagagagatgaggaagagacggagaaagaaagaggcggagttggagatgtatgcggaaggacatggaaactctggcgatagaactgctaacaacaccaattggaagctgactggtgtgaaagaagccttgagtatcaatcttgctgctttcgagaagccattgcggaagctcacgtttgcggatcttcttcaggctaccaatggtttccataatgatagtctgattggttctggtgggtttggagatgtttacaaagcgattttgaaagatggaagcgcggtggctatcaagaaactgattcatgttagcggtcaaggtgatagagagttcatggcggagatggaaaccattgggaagatcaaacatcgaaatcttgtgcctcttcttggttattgcaaagttggagacgagcggcttcttgtgtatgagtttatgaagtatggaagtttagaagatgttttgcacgaccccaagaaagctggggtgaaactaaactggtccacacggcggaagattgcgataggatcagctagagggcttgctttccttcaccacaactgcagtccgcatatcatccacagagacatgaaatccagtaatgtgttgcttgatgagaatttggaagctcgggtttcagattttggcatggcgaggctgatgagtgcgatggatacgcatttaagcgtcagtacattagctggtacaccgggttacgttcctccagagtattaccaaagtttcaggtgttcaacaaaaggagacgtttatagttacggtgtggtcttactcgagctactcacgggtaaacggccaacggattcaccggattttggagataacaaccttgttggatgggtgaaacagcacgcaaaactgcggattagcgatgtgtttgaccccgagcttatgaaggaagatccagcattagagatcgaacttttacaacatttaaaagttgcggttgcgtgtttggatgatcgggcttggagacgaccgacaatggtacaagtcatggccatgtttaaggagatacaagccgggtcagggatagattcacagtcaacgatcagatcaatagaggatggagggttcagtacaatagagatggttgatatgagtataaaagaagttcctgaaggaaaattatga

SEQ ID NO: 17 - protein, 335 aa, Arabidopsis thaliana
MTSDGATSTSAAAAAAAMATRRKPSWRERENNRRRERRRRAVAAKIYTGLRAQGNYNLPKHCDNNEVLKALCSEAGWVVEEDGTTYRKGHKPLPGDMAGSSSRATPYSSHNQSPLSSTFDSPILSYQVSPSSSSFPSPSRVGDPHNISTIFPFLRNGGIPSSLPPLRISNSAPVTPPVSSPTSRNPKPLPTWESFTKQSMSMAAKQSMTSLNYPFYAVSAPASPTHHRQFHAPATIPECDESDSSTVDSGHWISFQKFAQQQPFSASMVPTSPTFNLVKPAPQQLSPNTAAIQEIGQSSEFKFENSQVKPWEGERIHDVAMEDLELTLGNGKAHS

SEQ ID NO: 18 - DNA, 1008 bp, Arabidopsis thaliana
atgacgtctgacggagcaacgtcgacgtcagctgcagctgcagcagcagcgatggcgacgaggaggaaaccgtcgtggagagagagggagaacaatcggagaagagagcggcggagaagagctgttgcggcgaagatttatactggtcttagagctcaaggtaactacaatcttccaaaacattgtgacaacaatgaggttcttaaggctctttgttctgaagctggttgggttgttgaagaagacggaactacttatcgcaagggacacaagcctctacctggtgacatggctggatcatcttctcgagcaactccttactcttcccataaccaaagtcctctttcttccacttttgatagccccatcttatcttaccaagtcagtccttcctcttcttcattcccgagtccttctcgagttggtgatccacacaatatctccacaatcttccctttcctcaggaatggtggtattccttcatcgcttcctccacttagaatctcaaacagtgctcctgtcactccaccagtgtcatccccaacttctagaaaccccaaaccattgcctacttgggaatcttttaccaaacaatccatgtccatggctgctaaacagtcaatgacttctttgaactacccgttttatgcggtgtctgcacctgccagtcctactcatcatcgccagttccatgctccggctactatacctgaatgtgatgagtctgactcttccactgttgattctggtcattggataagctttcaaaagtttgcacaacaacagccattctctgcctctatggtgccaacctcgcctaccttcaatctcgtgaaacctgcaccacagcaattgtctccaaacacagcagcaatccaagagattggtcaaagctccgagtttaagtttgagaacagccaagttaagccatgggaaggggagaggatccatgatgtggctatggaggatctagagctcacgcttggaaatggtaaagctcatagttga

SEQ ID NO: 19 - protein, 267 aa, Arabidopsis thaliana
MEASPNDRLHFGKMGFGCKHYKRRCQIRAPCCNEVFDCRHCHNESTSTLRNIYDRHDLVRQDVKQVICSVCDTEQPAAQVCSNCGVNMGEYFCSICIFYDDDTEKQQFHCDDCGICRVGGRENFFHCKKCGSCYAVGLRNNHRCVENSMRHHCPICYEYLFDSLKDTNVMKCGHTMHVECYNEMIKRDKFCCPICSRSVIDMSKTWQRLDEEIEATAMPSDYRDKKVWILCNDCNDTTEVHFHIIGQKCGHCRSYNTRAIAPPVLPQ

SEQ ID NO: 20 - DNA, 804 bp, Arabidopsis thaliana
atggaagcttcacccaatgatcgacttcattttggcaaaatgggtttcgggtgtaagcattacaagaggagatgccaaatcagagctccatgttgcaacgaagtcttcgattgtcgccattgtcacaacgagagcactagcacattgcgcaatatctacgaccgtcacgatcttgttcgtcaagacgttaaacaagtgatttgttctgtttgcgatacagagcagccggcagctcaagtttgttcgaattgtggtgtcaacatgggagaatatttttgcagcatctgcatattctatgatgatgatactgaaaaacaacagtttcattgcgatgactgtggaatttgcagagttggtgggcgtgagaatttcttccattgcaagaagtgtggatcttgttatgcggttggtctgcgcaacaaccatcgctgcgttgagaattcaatgcgtcatcactgtcccatttgttacgagtacctttttgactctctaaaggacacaaatgtgatgaaatgcgggcacacaatgcacgtagaatgctacaacgagatgatcaaacgtgacaagttttgttgtccaatttgctcgaggtcagtgattgatatgtctaaaacatggcagagactcgatgaagagatcgaagccactgctatgccttcagattaccgtgacaagaaggtttggatactttgcaacgattgtaacgacacaacagaagtgcacttccacataatcggacagaaatgtggacattgcagatcatacaacacacgagcgattgcgcctcctgttcttcctcaatga

SEQ ID NO: 21 - protein, 407 aa, Arabidopsis thaliana
MQMDSPKSPLQPPTYGNLVTILSIDGGGIRGLIPAVILGFLESELQKLDGEEARLADYFDVIAGTSTGGLVTAMLTAPNKEGRPLFAASEIKDFYLEQCPKIFPQDHFPFSAAKKLVKSLTGPKYDGKYLHQLIHAKLGDTKLSQTLTNVVIPTFDIKHLQPTIFSSYEVKNHPLKDATLADIAISTSAAPTYLPAHFFKVEDLNGNAKEYNLIDGGVAANNPALLAIGEVTNEISGGSSDFFPIRPNDYGRFLVLSLGTGNHKAEEKFNAKEVAGWGLLNWLTHDNSTPIIDAFSQASSDMVDFHLSAVFRALHSEANYIRIQDDTLTGDAASVDIATVENLDILAKTGDELLKKPVARVNLDSGCNENAYETTNEHALIKLAGILSKEKKIRDIRSPHAKAPIRI

SEQ ID NO: 22 - DNA, 1224 bp, Arabidopsis thaliana
atgcaaatggacagccccaaatctcctctccagcccccgacctatggaaacttagttacaatcctcagcatcgacggtggtggcattagagggctaatccctgccgttatccttggttttctcgagtccgaactccagaaattggatggagaagaagcaaggcttgcagactactttgatgtaatagcgggaacaagcaccggtggtctagtgacagccatgctcaccgcgcctaataaggaaggccgacccttatttgcagcgtctgaaattaaagatttctatcttgagcaatgtccgaagatcttccctcaagatcatttcccattctcagccgccaaaaaactcgtgaagtccttgactggtcctaaatatgacggtaaataccttcatcagcttatccacgctaagttgggtgatacaaagttgagtcaaacacttaccaacgttgtcattccaacgttcgatatcaagcatcttcaacctactatctttagtagttatgaggtaaaaaaccatcctctcaaggacgcaaccctcgcagacattgccatctcaacttcagctgcccctacatacttgcctgcccatttcttcaaagttgaagatttaaacggaaacgctaaagaatacaatcttattgatggtggagttgcagctaacaacccggctttgttggccattggggaagtaacaaatgagatctcaggaggaagcagtgactttttcccaataagaccaaatgattacggaaggtttcttgtgctttcgcttggaaccggaaatcataaagccgaagagaaattcaatgcaaaagaagtagctggttggggactattgaattggttaacacacgacaactctacacctatcattgatgctttctcgcaagctagctccgacatggttgatttccatctctctgccgtttttcgagctcttcattccgaagccaactatattcgcatccaggatgacacattaactggggatgctgcttctgttgatatcgctaccgtcgagaatctggacattcttgccaagacaggagatgaactacttaaaaaacctgttgcaagagtcaacctagactcgggttgtaacgaaaatgcttatgaaacgactaatgaacatgctcttataaagttagcaggaatactttcaaaagaaaagaagatccgagacattcgttcacctcatgcaaaagctccaattaggatctaa

SEQ ID NO: 23 - protein, 528 aa, Arabidopsis thaliana
MERTNSIEMDRERLTAEMAFRDSSSAVIRIRRRLPDLLTSVKLKYVKLGLHNSCNVTTILFFLIILPLTGTVLVQLTGLTFDTFSELWSNQAVQLDTATRLTCLVFLSFVLTLYVANRSKPVYLVDFSCYKPEDERKISVDSFLTMTEENGSFTDDTVQFQQRISNRAGLGDETYLPRGITSTPPKLNMSEARAEAEAVMFGALDSLFEKTGIKPAEVGILIVNCSLFNPTPSLSAMIVNHYKMREDIKSYNLGGMGCSAGLISIDLANNLLKANPNSYAVVVSTENITLNWYFGNDRSMLLCNCIFRMGGAAILLSNRRQDRKKSKYSLVNVVRTHKGSDDKNYNCVYQKEDERGTIGVSLARELMSVAGDALKTNITTLGPMVLPLSEQLMFLISLVKRKMFKLKVKPYIPDFKLAFEHFCIHAGGRAVLDEVQKNLDLKDWHMEPSRMTLHRFGNTSSSSLWYEMAYTEAKGRVKAGDRLWQIAFGSGFKCNSAVWKALRPVSTEEMTGNAWAGSIDQYPVKVVQ

SEQ ID NO: 24 - DNA, 1587 bp, Arabidopsis thaliana
atggagagaacaaacagcattgagatggatcgagagagattaacggcggagatggcgtttcgagattcatcatcggccgttataagaattcgaagacgtttgccggatttattaacgtccgttaagctcaaatacgtgaagcttggacttcacaactcttgcaacgtgaccaccattctcttcttcttaattattcttcctttaaccggaaccgtgctggttcagctaaccggtctaacgttcgatacgttctctgagctttggtctaaccaggcggttcaactcgacacggcgacgagacttacctgcttggttttcctctccttcgttttgaccctctacgtggctaaccggtctaaaccggtttacctagtggatttctcctgctacaaaccggaagacgagcgtaaaatatcagtagattcgttcttgacgatgactgaggaaaatggatcattcaccgatgacacggttcagttccagcaaagaatctcgaaccgggccggtttgggagacgagacgtatctgccacgtggcataacttcaacgcccccgaagctaaatatgtcagaggcacgtgccgaagctgaagccgttatgtttggagccttagattccctcttcgagaaaaccggaattaaaccggccgaagtcggaatcttgatagtaaactgcagcttattcaatccgacgccgtctctatcagcgatgatcgtgaaccattacaagatgagagaagacatcaaaagttacaacctcggaggaatgggttgctccgccggattaatctcaatcgatctcgctaacaatctcctcaaagcaaaccctaattcttacgctgtcgtggtaagcacggaaaacataaccctaaactggtacttcggaaatgaccggtcaatgctcctctgcaactgcatcttccgaatgggcggagctgcgattctcctctctaaccgccgtcaagaccggaagaagtcaaagtactcgctggtcaacgtcgttcgaacacataaaggatcagacgacaagaactacaattgcgtgtaccagaaggaagacgagagaggaacaatcggtgtctctttagctagagagctcatgtctgtcgccggagacgctctgaaaacaaacatcacgactttaggaccgatggttcttccattgtcagagcagttgatgttcttgatttccttggtcaaaaggaagatgttcaagttaaaagttaaaccgtatattccggatttcaagctagctttcgagcatttctgtattcacgcaggaggtagagcggttctagacgaagtgcagaagaatcttgatctcaaagattggcacatggaaccttctagaatgactttgcacagatttggtaacacttcgagtagctcgctttggtatgagatggcttataccgaagctaagggtcgggttaaagctggtgaccgactttggcagattgcgtttggatcgggtttcaagtgtaatagtgcggtttggaaagcgttacgaccggtttcgacggaggagatgaccggtaatgcttgggctggttcgattgatcaatatccggttaaagttgtgcaatga

SEQ ID NO: 25 - protein, 550 aa, Arabidopsis thaliana
MGRSNEQDLLSTEIVNRGIEPSGPNAGSPTFSVRVRRRLPDFLQSVNLKYVKLGYHYLINHAVYLATIPVLVLVFSAEVGSLSREEIWKKLWDYDLATVIGFFGVFVLTACVYFMSRPRSVYLIDFACYKPSDEHKVTKEEFIELARKSGKFDEETLGFKKRILQASGIGDETYVPRSISSSENITTMKEGREEASTVIFGALDELFEKTRVKPKDVGVLVVNCSIFNPTPSLSAMVINHYKMRGNILSYNLGGMGCSAGIIAIDLARDMLQSNPNSYAVVVSTEMVGYNWYVGSDKSMVIPNCFFRMGCSAVMLSNRRRDFRHAKYRLEHIVRTHKAADDRSFRSVYQEEDEQGFKGLKISRDLMEVGGEALKTNITTLGPLVLPFSEQLLFFAALLRRTFSPAAKTSTTTSFSTSATAKTNGIKSSSSDLSKPYIPDYKLAFEHFCFHAASKVVLEELQKNLGLSEENMEASRMTLHRFGNTSSSGIWYELAYMEAKESVRRGDRVWQIAFGSGFKCNSVVWKAMRKVKKPTRNNPWVDCINRYPVPL

SEQ ID NO: 26 - DNA, 1653 bp, Arabidopsis thaliana
atgggtagatccaacgagcaagatctgctctctaccgagatcgttaatcgtgggatcgaaccatccggtcctaacgccggctcaccaacgttctcggttagggtcaggagacgtttgcctgattttcttcagtcggtgaacttgaagtacgtgaaacttggttaccactacctcataaaccatgcggtttatttggcgaccataccggttcttgtgctggtttttagtgctgaggttgggagtttaagcagagaagagatttggaagaagctttgggactatgatcttgcaactgttatcggattcttcggtgtctttgttttaaccgcttgtgtctacttcatgtctcgtcctcgctctgtttatcttattgatttcgcttgttacaagccctccgatgaacacaaggtgacaaaagaagagttcatagaactagcgagaaaatcagggaagttcgacgaagagacactcggtttcaagaagaggatcttacaagcctcaggcataggcgacgagacatacgtcccaagatccatctcttcatcagaaaacataacaacgatgaaagaaggtcgtgaagaagcctctacagtgatctttggagcactagacgaactcttcgagaagacacgtgtaaaacctaaagacgttggtgtccttgtggttaactgtagcattttcaacccgacaccgtcgttgtccgcaatggtgataaaccattacaagatgagagggaacatacttagttacaaccttggagggatgggatgttcggctggaatcatagctattgatcttgctcgtgacatgcttcagtctaaccctaatagttatgctgttgttgtgagtactgagatggttgggtataattggtacgtgggaagtgacaagtcaatggttatacctaattgtttctttaggatgggttgttctgccgttatgctctctaaccgtcgtcgtgactttcgccatgctaagtaccgtctcgagcacattgtccgaactcataaggctgctgacgaccgtagcttcaggagtgtgtaccaggaagaagatgaacaaggattcaaggggttgaagataagtagagacttaatggaagttggaggtgaagctctcaagacaaacatcactaccttaggtcctcttgtcctacctttctccgagcagcttctcttctttgctgctttgctccgccgaacattctcacctgctgccaaaacgtccacaaccacttccttctctacttccgccaccgcaaaaaccaatggaatcaagtcttcctcttccgatctgtccaagccatacatcccggactacaagctcgccttcgagcatttttgcttccacgcggcaagcaaagtagtgcttgaagagcttcaaaagaatctaggcttgagtgaagagaatatggaggcttctaggatgacacttcacaggtttggaaacacttctagcagtggaatctggtatgagttggcttacatggaggccaaggaaagtgttcgtagaggcgatagggtttggcagatcgctttcggttctggttttaagtgtaacagtgtggtgtggaaggcaatgaggaaggtgaagaagccaaccaggaacaatccttgggtggattgcatcaaccgttaccctgtgcctctctaa

SEQ ID NO: 27 - protein, 330 aa, Arabidopsis thaliana
MGRPPCCEKIEVKKGPWTPEEDIILVSYIQQHGPGNWRSVPANTGLLRCSKSCRLRWTNYLRPGIKRGNFTQPEEKMIIHLQALLGNRWAAIASYLPQRTDNDIKNYWNTHLKKKLVMMKFQNGIINENKTNLATDISSCNNNNNGCNHNKRTTNKGQWEKKLQTDINMAKQALFQALSLDQPSSLIPPDPDSPKPHHHSTTTYASSTDNISKLLQNWTSSSSSKPNTSSVSNNRSSSPGEGGLFDHHSLFSSNSESGSVDEKLNLMSETSMFKGESKPDIDMEATPTTTTTDDQGSLSLIEKWLFDDQGLVQCDDSQEDLIDVSLEELK

SEQ ID NO: 28 - DNA, 993 bp, Arabidopsis thaliana
atgggtagaccaccttgttgcgagaagattgaggtgaagaaaggaccatggactcccgaagaagacataatcttggtctcttatatccaacaacacggccctggaaattggagatctgtccctgcaaacaccggtttgctaaggtgtagcaagagttgcagacttagatggactaattaccttcgtcccgggatcaaacgaggaaatttcactcaaccggaagagaagatgatcatccaccttcaagctcttttgggaaatagatgggcagctatagcatcatatctacctcagaggaccgacaatgatatcaagaactactggaacactcatcttaaaaagaaactagtgatgatgaagtttcaaaatggtatcatcaacgaaaacaaaaccaatctggcaacagatatttcgtcttgtaataataacaacaatggatgtaatcacaacaaaaggaccaccaacaaaggccaatgggagaaaaaacttcaaacagacatcaacatggccaaacaagccttattccaagccttgtcacttgaccaaccatcttcattgatccctcccgatcctgactcaccaaaacctcatcatcattctaccaccacttatgcctcaagcacagataacatctctaaattactccagaactggacaagctcatcatcgtcaaagcctaacacttcatcagtctccaacaaccggagctcaagccccggtgaaggaggactttttgatcatcactctttgttctcatcgaattcagaatctggatcagttgatgagaagctgaatttgatgtccgagacaagcatgttcaaaggtgagagcaagccagacatagacatggaagctacacctactactactactactgatgatcaaggctcgttgtcattgatcgagaaatggttgtttgatgatcaaggcttggttcagtgtgatgatagtcaagaagatctcatcgacgtgtctttagaggagttaaaataa

SEQ ID NO: 29 - protein, 280 aa, Arabidopsis thaliana
MGRPPCCDKIGIKKGPWTPEEDIILVSYIQEHGPGNWRSVPTNTGLLRCSKSCRLRWTNYLRPGIKRGNFTPHEEGMIIHLQALLGNKWASIASYLPQRTDNDIKNYWNTHLKKKLNKSDSDERSRSENIALQTSSTRNTINHRSTYASSTENISRLLEGWMRASPKSSTSTTFLEHKMQNRTNNFIDHHSDQFPYEQLQGSWEEGHSKGINGDDDQGIKNSENNNGDDVHHEDGDHEDDDDHNATPPLTFIEKWLLEETSTTGGQMEEMSHLMELSNML

SEQ ID NO: 30 - DNA, 843 bp, Arabidopsis thaliana
atgggtaggcctccatgctgtgacaagatagggatcaagaaaggaccatggactcctgaagaagatatcattcttgtttcttacattcaagaacatggtcctggaaactggagatcagttcccaccaacactgggttattgagatgcagcaaaagttgtagactgagatggacaaattatctgagacctggaattaaacgtggaaactttactcctcatgaagaaggaatgatcattcacttgcaagccttattgggtaacaaatgggcgtccatagcttcatacctaccacaaagaacggacaatgatatcaagaactactggaacacacatttaaagaagaagctcaacaagtctgacagtgatgagaggagcagatcagagaacattgcgctgcaaacttcttcgacaagaaacaccattaatcatagatctacctatgcttcaagcaccgaaaacatttcccgccttcttgagggttggatgagagcgtctccaaagagtagtacaagtactactttcttggaacacaaaatgcagaaccggacaaacaatttcatcgatcatcacagcgatcagtttccatacgagcagcttcaaggttcttgggaagagggtcatagcaaaggaatcaacggggatgatgaccagggtataaagaattcagagaataacaacggtgatgatgttcatcatgaagatggtgatcatgaggatgatgatgatcataatgctacaccaccattgacatttattgagaaatggcttttggaggaaacaagtactactgggggtcaaatggaagagatgagccacttgatggagctctctaatatgctttaa

SEQ ID NO: 31 - protein, 333 aa, Arabidopsis thaliana
MGRPPCCDKIGVKKGPWTPEEDIILVSYIQEHGPGNWRSVPTHTGLRRCSKSCRLRWTNYLRPGIKRGNFTEHEEKMILHLQALLGNRWAAIASYLPERTDNDIKNYWNTHLKKKLKKMNDSCDSTINNGLDNKDFSISNKNTTSHQSSNSSKGQWERRLQTDINMAKQALCDALSIDKPQNPTNFSIPDLGYGPSSSSSSTTTTTTTTRNTNPYPSGVYASSAENIARLLQNFMKDTPKTSVPLPVAATEMAITTAASSPSTTEGDGEGIDHSLFSFNSIDEAEEKPKLIDHDINGLITQGSLSLFEKWLFDEQSHDMIINNMSLEGQEVLF

SEQ ID NO: 32 - DNA, 1002 bp, Arabidopsis thaliana
atgggaagaccaccatgctgtgacaagattggagtgaagaaaggaccatggacaccagaggaagatatcatcttggtttcttacatccaagaacatggtcctggaaactggagatctgtgcctactcacacaggtttgaggagatgtagcaaaagctgtagattgaggtggactaattatcttcgacctgggatcaagcgtggaaatttcaccgagcatgaagagaagatgattctccatcttcaagctcttttgggaaacaggtgggcagctatagcatcatatcttccagaaaggacagacaatgatataaagaactattggaacactcatttgaagaaaaagctcaagaagatgaatgattcttgtgatagtactatcaacaatggccttgataataaagacttctccatatcaaacaaaaacactacctcacatcaaagcagcaactccagtaaaggtcaatgggagagaaggcttcagacagatatcaacatggctaaacaagctctttgtgatgccttgtctattgacaaaccacaaaacccaactaatttttctattcccgatcttggttatggtccatcatcttcttcgtcctctaccaccaccaccaccaccaccacgagaaacactaatccatacccatctggggtctatgcttcaagtgctgagaacattgctcgtttgcttcagaattttatgaaagacacaccaaagacctcggtgcccttgccggttgcagccaccgagatggctatcaccacggcagcttcgagccctagcacaaccgaaggagacggagaagggattgaccattctttgttcagcttcaactccatagatgaagctgaagagaagcctaaactaatagaccatgacattaatggtctaattacacaaggctctctttctttgttcgagaaatggctctttgatgagcaaagccacgatatgatcatcaataacatgtcactagagggtcaggaagtgttgttctag

SEQ ID NO: 33 - protein, 351 aa, Arabidopsis thaliana
MGRPPCCEKIGVKKGPWTPEEDIILVSYIQEHGPGNWRSVPTHTGLRCSKSCRLRWTNYLRPGIKRGNFTEHEEKTIVHLQALLGNRWAAIASYLPERTDNDIKNYWNTHLKKKLKKINESGEEDNDGVSSSNTSSQKNHQSTNKGQWERRLQTDINMAKQALCEALSLDKPSSTLSSSSSLPTPVITQQNIRNFSSALLDRCYDPSSSSSSTTTTTTSNTTNPYPSGVYASSAENIARLLQDFMKDTPKALTLSSSSPVSETGPLTAAVSEEGGEGFEQSFFSFNSMDETQNLTQETSFFHDQVIKPEITMDQDHGLISQGSLSLFEKWLFDEQSHEMVGMALAGQEGMF

SEQ ID NO: 34 - DNA, 1056 bp, Arabidopsis thaliana
atgggaagaccaccttgctgtgaaaagattggagtgaagaaagggccatggacaccagaggaagacatcatcttggtttcttacatccaagaacatggtcctggaaactggagatctgtcccaacacacacaggtttaagatgtagcaagagctgcagattgagatggactaattatcttcgacccggtattaagcgtggaaattttactgagcatgaagagaagacaattgttcatcttcaagcccttttaggcaacagatgggcagccatagcatcataccttccagaaaggacagacaatgatataaagaactattggaacactcacttgaagaagaagctcaaaaagattaatgaatctggtgaagaagataatgatggtgtctcttcatcaaacactagttcacaaaagaaccatcaaagcactaacaaaggtcaatgggaaagaagacttcagacagacattaacatggcaaaacaagctctttgtgaggccttgtctttagacaaaccatcatccactctttcatcatcttcatcattaccgacaccagtaatcacacaacaaaacatccgtaacttctcatcagctttgcttgaccgttgttatgatccatcctcttcttcttcatctaccacaaccaccactacaagcaacactactaatccatacccatcaggggtatatgcgtcaagtgctgagaacatcgcccggttgcttcaagatttcatgaaagacacacccaaggctttaactttatcatcttcatctccggtttcagagactggaccactcactgctgcagtctcggaagaaggtggagaagggtttgaacaatctttcttcagcttcaattcaatggacgaaactcaaaacttgactcaggagacaagcttcttccatgatcaagtgatcaaaccggaaataacaatggaccaagatcatggtctaatatcacaagggtctctgtctttgtttgagaaatggttatttgatgagcaaagccacgagatggttggtatggcactagcaggacaagaagggatgttctag

SEQ ID NO: 35 - protein, 307 aa, Oryza sativa
MGRPPCCEKEGVKKGPWTPEEDMVLASYVQEHGPGNWRAVPPRTGLLRCSKSCRLRWTNYLRPGIRRGGFSHHEERLILHLQALLGNRWAAIASYLPHRTDNDVKNFWNTHLKKKLALTSSSSSPPTPTTPLVARGQWERKLQTDIDLARRALRDALSVDDAASPAMISSGPPAPAAAAAYALSERNISVMLSGWAAPPPARKGLSACNPAAATTTPGGAAAESASTAGTSLELTADCCSGGGDSSASNCLPSSMLLACDDGDATATAAGVAPLSAIESWLLLDDSGEPQLALDEQLLDVALRNYAF

SEQ ID NO: 36 - DNA, 924 bp, Oryza sativa
atggggcggccgccgtgttgcgagaaggagggggtgaagaaggggccatggacgccggaggaggacatggtgctggcgtcgtacgtgcaggagcacggcccgggcaactggcgcgccgtgccgcccaggacgggcctcctccgctgcagcaagagctgccgcctccgctggaccaactacctccgcccgggcatccgccgcggcggcttctcccaccacgaggagcgcctcatcctccacctccaggccctcctcggcaaccgctgggccgccatcgcctcctacctcccccaccgcaccgacaacgacgtcaagaacttctggaacacccacctcaagaagaagctcgccctcacctcctcctcctcctcgccgccgacgccgacgacgccgctcgtcgccagggggcagtgggagcgcaagctgcagaccgacatcgacctcgccaggcgcgccctccgcgacgccctctccgtcgacgacgccgcctccccggcgatgatcagcagcgggccgcccgcgccggcggcggcggcggcgtacgccctgagcgagcgcaacatctccgtgatgctgagcggctgggcggcgccgcctccggccaggaaaggattgtcagcctgcaacccggcggcggcgacgacgacgcccggtggcgccgccgcggagagcgcgtccacggccgggacgtcgttggagctcaccgccgactgctgctccggcggcggcgactccagcgcgtccaactgcctgccgtcgtccatgctcctcgcgtgcgacgacggcgacgcgacggcgacggcggccggggtggcgccgctgtcggcgatcgagtcgtggctgctgctcgacgacagcggagagccgcagctggcactggacgagcagctgctggacgtagctctccgtaattacgctttctaa

SEQ ID NO: 37 - protein, 321 aa, Oryza sativa
MGRPPCCDKVGVKKGPWTPEEDLMLVSYIQEHGAGNWRAVPTNTGLMRCSKSCRLRWTNYLRPGIKRGNFTEQEEKLIVHLQALLGNRWAAIASYLPERTDNDIKNYWNTHLKKKLKKMQAAGGGEDSGAASEGGGGRGDGDGGGKSVKAAAPKGQWERRLQTDIHTARQALRDALSLDHPDPSPATAAAAATPAGSSAAYASSADNIARLLQGWMRPGGGGGGNGKGPEASGSTSTTATTQQQPQCSGEGAASASASASQSGAAAAATAQTPECSTETSKMATGGGAGGPAPAFSMLESWLLDDGGMGLMDVVPLGDPSF

SEQ ID NO: 38 - DNA, 966 bp, Oryza sativa
atggggaggccgccgtgctgcgacaaggtcggggtgaagaaggggccatggacgccggaggaggacctgatgctggtctcctacatccaggagcacggcgccggcaactggcgcgccgtgccgacgaacaccgggctgatgcgttgcagcaagagctgccggctccggtggacgaactacctcaggccggggatcaagcgggggaacttcaccgagcaggaggagaagctcatcgtccacctccaggctctcctcggcaaccggtgggcagcgatagcgtcgtacttgccggagaggacggacaacgacatcaagaactactggaacacgcacctcaagaagaagctcaagaagatgcaggccgccggaggtggggaagacagcggcgccgcctcggagggtggcggcggccgcggcgacggcgacggcggcgggaaaagcgtgaaggccgccgcacctaaggggcagtgggagcggcggctgcagacggacatccacacggcgcggcaggcgctgcgcgacgcgctctcgctcgaccaccccgacccgtcgccggcgacggcggcggcggcggcgacgccagcggggtcgtcggcggcgtacgcgtcgagcgcggacaacatcgcgcggctgctgcagggctggatgcgcccgggcggcggcggcggcggcaacggcaagggccccgaggcgtcggggtcgacctccacgacggcgacgacgcagcagcagccgcagtgctccggcgagggcgcggcatccgcgtccgcgtcggcgagccagagcggcgccgccgccgcggcgactgcccagacgccggagtgctcgacggagacgagcaagatggccaccggcggcggcgccggcggccccgcgccggcgttctcgatgctggagagctggctgctcgacgacggcggcatggggctcatggacgtggtgccattgggggaccccagtttttag

SEQ ID NO: 39 - protein, 310 aa, Oryza sativa
MGRPPCCVKAEVKKGPWTPEEDLMLVAYVQEHGPGNWRAVPTNTGLMRCSKSCRLRWTNYLRPGIKRGNFTDQEEKLIVHLQALLGNRWAAIASYLPERTDNDIKNYWNTHLKKKLKKMSATGGGGDDGEGGGAGEVKTRAAAPKGQWERRLQTDIHTARQALRDALSLDPSPPAKPLDSSSGATAPPSSQAATSYASSAENIARLLEGWMRPGGGGGKTTTTPSSGSRSSAASVLSGEASHSGGATAPTPDGSTVTSKTKDEETAGAPPPPPPPAFSMLESWLLDDGMGHGEVGLMDVVVPLGDPSEFF

SEQ ID NO: 40 - DNA, 933 bp, Oryza sativa
atggggaggccgccgtgctgcgtgaaggcggaggtgaagaaggggccgtggacgccggaggaggacctcatgctcgtcgcctacgtccaggagcacggcccgggcaactggcgcgccgtgcccaccaacaccgggctgatgcggtgcagcaagagctgccggctccggtggaccaactacctccggccggggatcaagcgggggaacttcaccgaccaggaggagaagctcatcgtccacctccaggccctcctcggcaacaggtgggcggcgatcgcgtcctacttgccggagaggacggacaacgacatcaagaactactggaacacgcacctcaagaagaagctcaagaagatgagcgccaccggcggcggcggcgacgacggcgagggaggtggcgccggcgaggtgaagaccagggcggctgcgccgaaggggcagtgggagcggcgcctgcagacggacatccacaccgcgcgccaggcgctccgcgacgcgctctcgctggacccctcgccgccggccaagccgctggactcgtcgtcgggcgccacggcgccgccgtcgtcgcaggcggcgacgtcgtacgcatccagcgccgagaacatcgcgcggctcctggagggctggatgcgccccggcggcggcggcggcaagacgacgacgacgccgtcctccgggtcgaggtcgtcggcggcgtcggtgctgtccggggaggccagccacagcggcggcgccacggcgccgacgcccgacggctcgacggtcaccagcaagacgaaggacgaggagaccgccggcgcgccgccgccgccgccgccgccggcgttctccatgctggagagctggctgctcgacgacggcatgggccacggcgaggtggggctcatggacgtggtggtgccattgggggacccgagtgagttcttttag

SEQ ID NO: 41 - protein, 330 aa, Oryza sativa
MGRPPCCDNGVGVKKGPWTPEEDIILVSYIQQHGPGNWRSVPENTGLMRCSKSCRLRWTNYLRPGIKRGNFTPHEEGIIIHLQALLGNKWAAIASYLPQRTDNDIKNYWNTHLKKKVKRLQQQQQSHPDHHHHHSFQTTPSSSNAAAVATTSPNYYNPNNSNSNSSNYLHNNNHNLESMQSMATAPSNEATTIPKLFQFQTWMKPSPATTSSAATAAAGSCYKQAMAMQELQEEQEGSAAAAAMASSIDGVSKDQDYHMCAVISGDDKSSSSEMMTAAAMAGHGEAATTTFSLLENWLLDDMPGQAAMSAAMDGFLEISAGYCCADPIMF

SEQ ID NO: 42 - DNA, 993 bp, Oryza sativa
atggggagacctccatgctgcgacaatggcgtcggcgtcaagaaagggccatggacgccagaggaggacatcatcctcgtctcctacatccagcagcatggccccgggaactggcgctccgtgcccgagaacaccggattgatgaggtgcagcaagagctgcaggctgcggtggacgaactacttgagaccggggatcaagcgtggcaacttcacccctcatgaggaggggatcatcatccacctccaggcattgcttggcaacaagtgggcagcaatagcctcctacctcccccaaagaacagacaacgacatcaagaactactggaacacacacctcaagaagaaggtgaagaggctgcaacaacaacaacaatcacaccctgatcatcatcaccaccattccttccaaaccaccccttcttcctccaatgcagcagcagtagcaacaaccagcccaaactactacaaccctaacaacagcaacagcaacagcagcaattacctccataacaacaaccacaatcttgaatccatgcaatccatggccactgcacctagcaatgaggccaccaccatccccaagctcttccagttccagacatggatgaagccatcaccagcaacaacatcatcagcagcaacagctgctgcaggtagctgctacaagcaggccatggccatgcaggagctccaagaggagcaagagggctctgctgctgctgctgcaatggcttcttccattgatggcgtctccaaggaccaggattatcacatgtgtgctgtgatcagtggtgatgacaagtcgtcgtcgtcggagatgatgacggctgcggcaatggccggccatggcgaggcggccacgacgaccttctcgctgctcgagaactggctgctcgacgacatgccggggcaggcggccatgagcgccgccatggatgggttcttggagatctctgctggatactgctgtgcagaccctatcatgttctga

SEQ ID NO: 43 - protein, 152 aa, Oryza sativa
MVRPPCCDKDGVKKGPWTPEEDLVLVSYVQEHGPGNWRAVPTRTGLMRCSKSCRLRWTNYLRPGIKRGNFTDQEEKLIVHLQALLGNRWAAIASYLPERTDNDIKNYWNTHLKRKLQGGDETQLSAIESWLFADADGIESGSLLDAAMDYTF

SEQ ID NO: 44 - DNA, 459 bp, Oryza sativa
atggtgaggccgccgtgctgcgacaaggacggcgtcaagaagggcccgtggacgccggaggaggacctcgtcctcgtctcctacgtccaggagcacggccccggcaactggcgcgccgtcccgaccagaacagggctgatgcggtgcagcaagagctgtaggctccggtggaccaactacctgaggcccgggatcaagcggggaaacttcaccgaccaggaggagaagctcatcgtccacctccaggcgctcctcggcaaccgctgggcggccatcgcgtcgtacctccccgagcgcacggacaacgacatcaagaactactggaacacccacctcaagcgcaagctgcagggcggcgacgagacgcagctctccgccatcgagtcgtggctgttcgccgacgccgacggcatcgagagtggcagcttgctcgacgcggccatggattacaccttctaa

SEQ ID NO: 45 - protein, 306 aa, Sorghum bicolor
MGRPPCCEKSGVKKGPWSPEEDLLLVSYVQEHGPENWRAVPSNTGLMRCSKSCRLRWTNYLRPGIKRGNFSDQEEKLIIELQALLGNKWSTIASYMRDRTDNDIKNYWNTHLRKKLAKTCASESGASGGSAKTKGDGAAAPAPAPKGQWERQLQTDMHTARQALQEALSIDTAPPPPAAIKPEPLPLAQLPAPALSPAMYACSIENVVRVLELWMQRSASEKASAQSMTSISAVSGGGEGGSGSQSGTARALEGFTGMTKVDGAGGAGPGPSSSLPMLESWLLDDGMGHGDEGLFCVPLADPCEFF

SEQ ID NO: 46 - DNA, 921 bp, Sorghum bicolor
atggggaggccgccgtgctgcgagaagagcggggtgaagaaggggccctggtcgccggaggaggacctcctgctcgtctcctacgtgcaggagcacggtcctgagaactggcgcgccgtgcctagcaacaccggtctgatgcgctgcagcaagagctgccggctccggtggaccaactacctccgcccgggcatcaagcgcggcaacttcagcgaccaggaggagaagctcatcatcgagctccaggcactgctcgggaacaagtggtccacgattgcgtcgtacatgcgggatcggacggacaatgacatcaagaactattggaacacgcacctgaggaagaagctcgccaagacgtgcgccagtgaaagcggtgcctccggcggctccgccaagacgaagggcgatggggcagccgcgcccgcgcccgcgcccaaggggcagtgggagcggcagctgcagacggacatgcacaccgcacgccaggctctccaggaggcgctgtccatcgacaccgcgccgccgccaccggcggccatcaagccggagccgctgccgctagcgcagctgccggcgccagccctcagcccggcgatgtacgcttgcagcatcgagaacgttgtgcgcgtgctggagctctggatgcaacgcagcgccagcgagaaggcgtcggcccagtcgatgacctccatctcggcggtctccggtggtggagagggcgggtcgggaagccagagcggcacggcgcgcgcgctggaggggttcaccgggatgacaaaggtagatggcgcgggtggcgcagggccggggccgtcgtcgtcattaccgatgctggagagctggctgctcgacgacggcatggggcatggtgacgagggcctcttctgcgtgccgctggcggacccgtgcgagttcttttag

SEQ ID NO: 47 - protein, 306 aa, Sorghum bicolor
MGRPPCCDKVGVKKGPWTPEEDLMLVSYVQEHGPGNWRAVPTNTVMRCSKSCRLRWTNYLRPGIKRGNFTDQEEKLIIHLQALLGNRWWAAIASYLPERTDNDIKNYWNTHLKKKLKKMQAGEGGGGGKRPAVPKGQWERRLQTDIHTARQALRDALSLEPSAQPLAPAKVEPLPTTPPGCTTYASSAENIARLLEGWLRPGGGGGKGPEASGSTSTTATTQQRPQCSGEGAASASASHSGGAAANTAAQTPECSTETSKMAGAAGSAPPAFSMLESWLLDDGGMGHGEVGLMTDVVPLGDPSEFF

SEQ ID NO: 48 - DNA, 921 bp, Sorghum bicolor
atggggcggccgccgtgctgcgacaaggtgggcgtgaagaaagggccgtggacccccgaggaggacctcatgctcgtctcctatgtccaggagcacggccccggcaactggcgcgccgtgccgaccaacaccgtgatgcggtgcagcaagagctgccggttgcggtggacgaactacctccggccgggaatcaagcgcggcaacttcaccgatcaggaggagaagctcatcatccacctccaggctctccttggcaacaggtggtgggcggcgatagcgtcctacttgccggagaggacggacaacgatatcaagaactactggaacacgcacctcaagaagaagctgaagaagatgcaggccggcgaagggggcgggggagggaagcgcccggccgtgcccaaggggcagtgggagcggcggctgcagaccgacatccacacggcgcggcaggccctgcgcgacgcgctctcgctggagccttcggcgcagccgctggcgccggcgaaggtggagcctctgccgacgactccgccggggtgcacgacgtacgcgtctagcgccgagaacatcgcgcggctgctggaggggtggctgcgccccggcggcggcgggggcaaggggccggaggcgtcgggttcgacgtcgacgacggccacgacgcagcagcggccgcagtgctccggtgagggcgccgcgtccgcgtcggcgagccacagtggtggggcggccgcgaacacggcggcgcagacccccgagtgctcgacggagaccagcaagatggccggcgcggctggctccgcgccgccggcgttctcgatgctggagagctggctgctcgacgacggcggcatggggcacggcgaggtggggctcatgaccgacgtggtgccattaggggaccccagtgagttcttttaa

SEQ ID NO: 49 - protein, 304 aa, Sorghum bicolor
MGRPPCCEKGGVKKGPWTPEEDLVLVSYVQDHGPGNWRAVPTSTGLMRCSKSCRLRWTNYLRPGIKRGNFSDQEEKLIIHLQALLGNRWAAIASYMPERTDNDIKNYWNTHLKKKFTKTGGGGGAEAKSGRCAAPKGQWERRLQTDIHTARQALREALSLDPDPVPPSAKPEQVPQQPPAPAATQAAGQATYASSAENIARLLEGWMHPGGGSGAAGKVSSGSRSSASSVSAFSGDEGASASNSGTAVRMPERPTRTSKAVDDAGTAGPGPSFSMLESWLLDDGVGHGDTGLVSVPLGDPCEFF

SEQ ID NO: 50 - DNA, 915 bp, Sorghum bicolor
atggggaggccaccgtgctgcgagaagggtggggtgaagaaggggccatggacgccggaggaggacctcgtgctcgtctcctatgtgcaggaccacggccccgggaactggcgcgccgtgcccaccagcaccgggctgatgcgatgcagcaagagctgccggctccggtggaccaactatctccgcccgggcatcaagcgcggcaacttcagcgaccaggaagagaagctcatcatccacctccaggcgctgctcgggaacaggtgggcggcgatcgcgtcgtacatgccggagcggacagacaacgacatcaagaactactggaacacgcacctcaagaaaaagttcactaagacgggcggcggcggtggcgccgaggcgaagagcggcagatgcgccgcgcccaaggggcagtgggagcggcggctgcagacggacatccacaccgcgcgccaagcactccgggaggcactgtccctggaccccgaccccgtgcctccgtccgccaagccggagcaagtgccgcaacaaccgccggcgccagctgctacccaggccgccggccaggcgacatacgcttccagcgccgagaacatcgcgcgcctgctggagggctggatgcaccccggcggcggctccggcgccgccgggaaggtgtcgtccgggtcgaggtcctcggcctcctccgtgtcggcgttctccggtgatgagggcgcgtcggcgagcaacagcggcacagcagtgcgcatgccagagaggcctaccaggacgagcaaggcggtggatgatgcgggcaccgcggggccggggccatcgttctcaatgctggagagctggctgctcgacgacggcgtggggcatggcgacacggggctcgtcagcgtgccgttgggcgacccgtgcgagttcttttag

SEQ ID NO: 51 - protein, 328 aa, Sorghum bicolor
MVRPPCCDKEGVKKGPWTPEEDLVLVSYIQEHGPGNWRAVPAKTGLMRCSKSCRLRWTNYLRPGIKRGNFTEQEEKLIIHLQALLGNRWAAIASYLPERTDNDIKNYWNTHLKRKLQSGGGDGAAKPPAHRPPSSSKGQWERRLQTDINLARRALREALTPLDDLKPPQLQRDATAVDAPGAGLGVGGGDSPASSSSGASQCSPSSAPAAAATAAGPYVLTTENISRMLDGWAGRKAARGGSPGTPGGAESASTGSSDASEVSYGGGAAVTLAAAGGPVFEFETKPTVPPAQQMPLSAIESWLFDDDSHFHHVQSAGVLDAAPMDYPF

SEQ ID NO: 52 - DNA, 987 bp, Sorghum bicolor
atggtgaggccgccgtgctgcgacaaggagggcgtcaagaagggcccctggacgccggaggaggacctcgtcctcgtctcctacatccaggagcacggccccggcaactggcgcgccgtcccggccaaaactgggctgatgcggtgcagcaagagctgccggctgcggtggaccaactacctccggccgggcatcaagcgcggcaacttcacggagcaggaggagaagctcatcatccacctccaggccctccttggcaacaggtgggcggccatcgcgtcgtacctgccggagcggacggacaacgacatcaagaactactggaacacgcacctcaagcgcaagctgcagagcggcggcggcgacggggcggccaagccgccggcgcacaggccgccgtcgtcgtccaagggccagtgggagaggaggctgcagacggacatcaacctggcgcgccgcgcgcttcgcgaggccctcaccccgctcgacgacctcaagccgccacagctgcagcgcgacgccaccgccgtcgacgcgccgggcgctggcctgggcgtgggcgggggcgacagcccggcgtcgagctcgtcgggcgcgtcgcagtgctccccgtcgtcggcgcctgccgctgccgccaccgccgcggggccgtacgtactgaccacggagaacatctcgcggatgctggacggctgggctggccggaaggccgcccgcggcggcagtccgggcacgcccggcggcgccgagagcgcgtccaccggatcctcggacgcgtcggaggtgtcgtacggtggcggcgccgccgtcacgctggcggcagctggcgggccggttttcgagttcgagacgaagccgaccgtgccgccggcccagcagatgccgctgtcggcgatcgagtcgtggctgttcgacgacgacagccacttccaccatgtccagagcgccggcgtgctcgatgcggcccccatggattacccgttctag

SEQ ID NO: 53 - protein, 296 aa, Sorghum bicolor
MGRPPCCDNGVGVKKGPWTPEEDIVLVSYIQQHGPGNWRSVPENTGLMRCSKSCRLRWTNYLRPGIKRGNFTPHEEGIIIHLQALLGNKWAAIASYLPQRTDNDIKNYWNTHLKKKVKRLQQPAAAESFQTTAAASNAVTCSPNYYSSSSSSHHSLQGMQQPMSSYPNTACSSSTPSNHETTTTTGVSDLFQTWMMRPSPLAAAAAAADNCKIAMQEFQEEQASIVCQEQMVMTGGGDVNNKSSALEMMVAPAVMGASTATFSLLEDWLLDDMPGQVAMDGLMGISAGCCADPIMF

SEQ ID NO: 54 - DNA, 891 bp, Sorghum bicolor
atggggaggccaccgtgctgcgacaacggcgtcggcgtcaagaaagggccatggacaccggaggaggacatcgtcctcgtctcctacatccagcagcacggcccggggaattggcggtccgtgccagagaacacagggctgatgaggtgcagcaagagttgcaggctgcggtggaccaactacctcaggcctgggatcaagcgtgggaacttcactcctcatgaggaagggatcatcatccacctccaggcgttgcttggcaacaagtgggcagccatagcctcgtacctccctcaaagaaccgacaacgacatcaagaactactggaacacacacctcaagaagaaggtgaagaggctgcaacaacctgctgcagccgagtccttccaaaccactgccgccgcctccaatgcagtcacctgcagcccaaactactacagctctagcagcagcagccaccacagcctccaaggaatgcagcagcccatgagcagctaccccaacaccgcctgcagcagcagcacaccaagcaaccatgagaccaccaccaccaccggcgtctccgacctcttccagacatggatgatgagaccatcaccactagcagcggcggcggcggcagcagataactgcaagatcgccatgcaagagttccaggaagaacaagcctccatcgtttgccaggaacagatggtgatgaccggcggcggtgatgttaacaacaagtcgtcggcgttggagatgatggtggcgccggcggtgatgggggcgagcaccgctaccttctcgctgctcgaggactggctgctcgatgacatgccggggcaggttgccatggatgggctcatggggatctctgccggttgctgtgcagatcccatcatgttctag

SEQ ID NO: 55 - protein, 356 aa, Sorghum bicolor
MGRPPCCDKEGIKKGPWTPEEDIILVSYIQEHGPGNWRSVPINTGLMRCSKSCRLRWTNYLRPGIRRGNFTPHEEGIIVHLQSLLGNRWAAIASYLPQRTDNDIKNYWNTHLKKKLKKHQAIGPSSRRRRLPHPTPPRPRPSSSCPPPPSAAAAEAAMSTTTTVTCSAAAPISSDSYYARPSGGAGCCSNPAEVAQLIARRSPFAADGGGDSSSSSYASSMDNISKLLTGFMKQQQSSPSPDAAAAADIKPSSAAHVNNHHALLSSSSSFHHMSAAGTGSGTPPAAACFNDMTMPSPPHVQQQAALMGHHGGYDDDPRQASPLSPIEKWLFEEAAEQVGDLMDLSEDCCSSVPMMF

SEQ ID NO: 56 - DNA, 1071 bp, Sorghum bicolor
atgggcaggccaccgtgctgcgacaaggaagggatcaagaaggggccatggacgccggaggaggacatcatcctggtgtcctacatccaggagcacggcccgggcaactggcgctccgttcccatcaacacggggctcatgcgctgcagcaagagctgccgcctccggtggaccaactacctccgccccggcatccgccgcggcaacttcaccccgcacgaggaaggcatcatcgtccacctccagtccttgctcggcaacaggtgggccgccattgcttcttacctcccgcagagaaccgacaacgacatcaagaactactggaacacccacctcaagaagaagctcaagaagcaccaggccatcggcccatcttcgcgccgccgccgcctcccgcatccgactcctcctcgtcctcgtccatcgtcgtcatgcccaccaccaccgtcggcggcggcggcggaggctgccatgtccaccaccaccaccgtgacatgctcggcggcggcaccaatctcctcggacagctactacgcgcgcccatcaggaggagcaggctgctgcagcaacccagccgaggtcgcccagctcatcgcccggcgctcgccgttcgccgccgacggtggtggcgacagctcctcgtcgtcgtacgcctccagcatggacaacatatccaagctgctcaccggcttcatgaagcagcagcagagctccccgtcccccgacgccgccgcagctgccgacatcaagccctcctcggccgcccatgtcaacaaccaccatgctctgctgtcgtcgtcgtcgtcgttccatcacatgtccgccgccggcaccgggagtggtacgccacctgcagcagcctgcttcaacgacatgacgatgccgtcgccgccgcatgtgcagcagcaggcggcgctgatggggcatcacggcggctacgacgacgaccccaggcaggcgtccccgctgtctccgatcgagaagtggctgttcgaagaggccgccgagcaggtcggcgacctcatggatctgtccgaagactgctgctcatcagttccgatgatgttttag

SEQ ID NO: 57 - protein, 315 aa, Sorghum bicolor
MGRPPCCDKVGIKKGPWTPEEDIVLVSYIQEHGPGNWRSVPINTGLMRCSKSCRLRWTNYLRPGIRRGNFTPHEEGIIVHLQSLLGNRWAAIASYLPQRTDNDIKNYWNTHLKKKLQKQQAIGAIFAPPPPPSESPIIPAVVPTATTGSHADCHHDDMMTLSKDSYGRPASSTPAPADEVTQFIGLCSPPFAATNGDTFSSPMDNISKLLNGFMMKSSPTQDDAATNIKPSSVIDINPFDHKSGGALSDDVPLLMPPPQQQQQQALAGHGGYHKPKLQQLSSIEKWLFDEAAEQVVDHQLMEISDGCCSVPSLLL

SEQ ID NO: 58 - DNA, 948 bp, Sorghum bicolor
atgggcaggccgccgtgctgcgacaaggtggggatcaagaaggggccatggacgccggaggaggacatcgtcctggtgtcctacatccaggagcacggccccggcaactggcgctccgtgcccatcaacacgggcctcatgcgctgcagcaagagctgccgcctccgctggaccaactacctccgccccggcatccgccgcggcaacttcaccccccacgaggaaggcatcatcgtccacctccagtccttgctcggcaacaggtgggccgccattgcttcttacctcccgcagagaaccgacaacgacatcaagaactactggaacacccacctcaagaagaagcttcagaagcagcaagccatcggcgccatcttcgcgccaccacctccgccctccgaatctcccatcataccggcggtagtacccaccgccaccaccggcagccatgctgattgccatcacgatgacatgatgaccctctccaaggacagctacgggcgcccagccagcagcacaccagctccggctgatgaggtcacccaattcatcggcctgtgctcgccgccgttcgctgccaccaatggtgacaccttctcgtcgcccatggacaacatatccaagctgctcaacggcttcatgatgaagagctccccgacacaggatgacgctgctaccaatatcaagccctcctcggtcatcgacatcaaccctttcgatcacaagtccggcggtgcactctccgacgacgtgccactgctgatgccaccaccgcagcagcagcagcagcaggcattggcgggacacggcggttaccacaagcccaagctgcagcagctgtcctccatagagaagtggttgttcgacgaggccgccgagcaggtcgtcgaccaccagctgatggagatctccgacggctgttgctcagttcccagcttgctgctttag

SEQ ID NO: 59 - protein, 307 aa, Vitis Vinifera
MGRPPCCDKIGVKKGPWTPEEDIILVSYIQEHGPGNWRAVPTSTGLLRCSKSCRLRWTNYLRPGIKRGNFTDQEEKTIIHLQALLGNRWAAIASYLPQRTDNDIKNYWNTHLKKKLKKFPTGVDDHNQDGFSISKGQWERRLQTDIHMAKQALCEALSIDTSSSLPDLKSSNGYNPNTRPVQASTYASSAENIAKLLEGWMRNSPKSTRTNSEATQNSKNSSEGATTPDALDSLFSFNSSNSDLSLSNDETANFTPETILFQDESKPNLETQVPLTMIEKWLFDEGAATQEQEDLIDMSLEDTAQLF

SEQ ID NO: 60 - DNA, 924 bp, Vitis Vinifera
atggggaggccaccttgctgtgacaagatcggggtgaagaaagggccatggactcctgaagaggacatcatcttggtctcttacattcaagaacatggtccagggaattggagagcagttcctactagcacaggtctgcttagatgcagtaagagttgcaggcttagatggactaattatctccgcccgggtatcaaacgcggtaactttactgatcaggaggagaagacgataatccacctccaggctcttttgggcaatagatgggctgccatagcttcttatcttcctcaaagaacggacaatgatataaaaaattattggaacacccatttgaaaaagaagctgaagaagtttcccacaggtgtagatgaccataatcaagatgggttttcaatctccaaaggtcagtgggagagaaggcttcaaacagacatccacatggctaaacaagcgctatgtgaggctttgtccatagatacgtcaagctcgctgcctgacttgaagagctctaacggctacaaccctaacaccagacctgtccaagcatctacatatgcatccagtgctgaaaacatagccaaattgctggaaggttggatgagaaattcaccaaaatcaactcgaacgaattctgaagctactcagaactccaaaaactccagtgaaggggcaactacaccagatgctcttgactcgttgtttagcttcaactcttccaactctgatctttctctgtctaatgatgagacagcaaatttcacacccgaaaccattctcttccaagatgaaagcaagccaaatttggagactcaagtccctctcacaatgatagagaaatggctctttgatgaaggtgctgctactcaagaacaagaagacctaattgacatgtcactagaggacactgctcagctcttctag

SEQ ID NO: 61 - protein, 327 aa, Vitis Vinifera
MGRPPCCDKIGVKKGPWTPEEDIILVSYIQEHGPGNWRAVPTNTGLLRCSKSCRLRWTNYLRPGIKRGNFTDHEERMIIHLQALLGNRWAAIASYLPQRTDNDIKNYWNTHLKKKLKKLQTGSSSDGHSRDVSLASQSISRGQWERRLQTDIHMAKQALHEALSLEKPHSSSDQLNPTNGYQTCTRPGQASSYASSTENIARLLEGWMRNSPKQARASSATTQNSFINTAGTDSTSSEGTPSAANNEDIELTEAYEMLFGFDSLDSSNSEISQSKSPEASFFQEESKPDLSDQVPLSFLEKWLFEEGGAQGKEDLTDISLDESPDFF

SEQ ID NO: 62 - DNA, 984 bp, Vitis Vinifera
atgggaagaccaccttgctgtgataaaatcggtgtgaagaaaggaccatggacacctgaagaagatatcatactagtctcttatatccaggaacatggtccggggaattggagggctgttcctactaatacaggattgcttagatgcagtaagagttgcaggcttagatggactaactacctccggcctggaatcaaaagaggtaactttaccgaccatgaggagaggatgatcattcaccttcaagctcttttgggcaacagatgggctgccatagcttcttatcttcctcagagaacagacaatgacatcaagaactattggaatacccatttgaagaaaaagctgaaaaagcttcaaacaggctcatcctcagatggtcactctagagatgtgtccctagcatcacagtcaatctcaagaggccagtgggagagaaggcttcaaactgatatccacatggctaagcaagctcttcatgaggccttgtctctggagaagccacacagttcctctgatcaattgaaccccactaatggctatcagacttgcacaagacctggtcaagcatcttcctatgcatccagtactgagaacattgctcggttgctagaagggtggatgagaaattcgcccaagcaagctagagcaagctcagctaccactcaaaattcattcatcaacactgctgggaccgattccacctctagtgaagggaccccaagtgcagcaaacaacgaagacattgaattaactgaagcatatgaaatgctctttggattcgactccttggactcttccaattctgaaatttcccaatctaaatcccctgaggcaagctttttccaggaagaaagcaagccggatctcagcgaccaagtgccattgtcattccttgagaagtggctatttgaagaaggcggtgctcaaggaaaagaagaccttactgatatctcattagatgaaagtcctgattttttctga

SEQ ID NO: 63 - protein, 279 aa, Vitis Vinifera
MGRPPCCDKVGIKKGPWTPEEDIILVSYIQEHGPGNWRSVPTNTGLLRCSKSCRLRWTNYLRPGIKRGNFTPHEEGMIIHLQALLGNKWAAIASYLPQRTDNDIKNYWNTHLKKKIKNYAGDDHHRRGSSFEVINGHSSAHPSLNSPISTYASSTENISRLLEGWMRSSPKATKEKLHQNSSLEEGSIDMTGNSMAQGGGELVANDEFESILEYENLNDDHHQTTDATIPSDDHDHDHEMKMDHDQKKHNPPLSFLEKWLLDESAAQGEEMMDQLSPIF

SEQ ID NO: 64 - DNA, 840 bp, Vitis Vinifera
atgggaaggcctccttgctgtgataaagttggtatcaagaagggtccttggaccccagaagaggacatcatcttggtctcctatatccaagagcatggccccggaaattggagatcagtgcctacaaacaccgggctgctgaggtgtagcaagagttgcaggcttagatggactaattaccttagaccggggattaagcgcggtaacttcactccccatgaagaaggaatgatcatccatctacaagccctattgggtaacaaatgggctgccatagcttcatacctccctcaaagaactgataatgatattaagaattattggaacactcacttgaagaagaagatcaagaactatgctggtgatgatcatcacagaaggggtagttcttttgaggtcatcaatggccattcctcggctcacccgagcctaaacagcccgatctcgacgtatgcctccagcactgagaacatctcaaggctactagaaggttggatgaggtcctccccaaaggccaccaaagagaaactgcaccaaaacagcagcttggaagaaggtagtatcgatatgaccggaaactccatggcgcaaggagggggcgaattggtcgccaacgacgagtttgagtccattcttgagtacgaaaacctgaatgatgatcatcatcagactactgatgctactattccaagtgatgatcatgatcatgatcatgagatgaagatggatcatgatcagaagaagcacaaccctcctctatcatttcttgagaaatggctcttggatgaatcagcagctcaaggagaggagatgatggatcaactctctccaatattctga

SEQ ID NO: 65 - protein, 322 aa, Populus trichocarpa
MGRPPCCDKIGVKKGPWTPEEDIILVSYIQEHGPGNWRAVPTSTGLLRCSKSCRLRWTNYLRPGIKRGNFTDHEEKMIIHLQALLGNRWAAIASYLPQRTDNDIKNFWNTHLKKKLRKLQAGQEGQSRDGLSSTGSQQISRGQWERRLQTDINMARQALCEALSPGKPSSLLTGLKPSCGYEKPATEPIYASSTENISRLLKGWMISGPKQSLKNSTTQNSFIDTAGADSLSSEGTPDKADKNGTGLSQAFESLFGFDSFDSSNSDFSQSMSPDTGLFQDESKPNSSAQVPLSLIERWLFDEGAMQGKDYINEVTIDEDNLF

SEQ ID NO: 66 - DNA, 969 bp, Populus trichocarpa
atgggcagaccaccttgctgtgataagataggagtgaaaaaaggaccatggactcctgaggaagatatcatcttggtatcatatattcaagaacatggtcctgggaattggagagctgtgccaactagtacaggactgcttagatgcagtaagagttgcagactgagatggactaattacctaaggccagggatcaaacgtggtaattttaccgatcacgaggagaagatgataatccacctccaagcccttctaggcaacagatgggctgccatagcttcatacctccctcagagaacagataatgacattaaaaacttttggaacacacatttgaagaagaagttgagaaagcttcaagcagggcaagaaggtcagtctagagatgggttatcatcaacaggttcacagcaaatttctagaggccaatgggagagaaggcttcaaactgatatcaacatggctaggcaagccctatgcgaggccttgtctcccggtaaaccaagcagcttgttaaccgggttgaaaccctcttgtgggtatgaaaaaccagctacagaaccaatctatgcatcaagcactgaaaatatatccagattgctcaaaggatggatgataagtgggcctaagcagtcgctaaaaaattcaactactcagaattccttcatcgatacggctggagctgattcactgtctagtgaagggactcctgataaagcagacaaaaatggcactggattatcacaggcatttgaatcactctttggttttgactctttcgactcttcaaattcagatttctctcaatccatgtcgcctgatactggccttttccaagacgaaagtaagccaaattccagtgctcaagtgccactgtcattgattgagaggtggctatttgatgaaggagccatgcaagggaaagattacataaacgaagtcacaatagatgaagataatctcttctag

SEQ ID NO: 67 - protein, 332 aa, Glycine max
MGRPPCCDKEGVKKGPWTPEEDIILVSYIQEHGPGNWRAVPAKTGLSRCSKSCRLRWTNYLRPGIKRGNFTEQEEKMIIHLQDLLGNRWAAIASYLPQRTDNDIKNYWNTHLRKKLKKMQAGGEGGSFGEGFSASRQIPRGQWERRLQTDIQMAKRALSEALSPEKKPSCLSASNSNPSDSSSSFSSTKPTTTQSVCYASSADNIARMLKGWMKNPPKSSRTNSSMTQNSFNNLAGADTACSSGAKGPLSSAELSENNFESLFDFDQSLESSNSDQFSQSLSPEATVLQDESKPDINIAAEIMPFSLLEKWLLDEAGCQEKLVGCCGDAKFF

SEQ ID NO: 68 - DNA, 999 bp, Glycine max
atgggaagaccaccttgttgtgacaaagaaggggtcaagaaagggccttggactcctgaagaagacatcatattggtgtcttatattcaggaacatggtcctggaaattggagggcagttcctgccaaaacagggttgtcaagatgcagcaagagttgcagacttagatggacgaattacctgaggccaggaatcaagcgtggtaacttcacagaacaagaggagaagatgataatccatcttcaagatcttttaggaaacagatgggctgcaatagcttcataccttccacaaagaacagacaatgacataaagaactattggaatacccatttgagaaagaagctgaagaagatgcaagcaggcggtgaaggtggtagctttggagaagggttttcagcctcaaggcaaatccctagaggccagtgggaaagaaggctccaaactgatatccaaatggcaaagagagccctcagtgaagctctttcaccagagaaaaagccatcttgtttatctgcctcaaactcaaacccttcagatagtagcagctccttctcttccacaaaaccaacaacaacacaatctgtgtgctatgcatcaagtgctgacaacatagctagaatgctcaagggttggatgaagaacccaccaaagtcctcaagaaccaactcgtctatgactcagaactcattcaacaacttagcaggtgctgatactgcttgtagtagtggagcaaagggaccactaagcagtgccgaattgtctgagaataattttgaatccttgtttgattttgatcagtctttggagtcttcaaactctgatcaattctctcagtccttgtctcctgaggccactgttttgcaagatgaaagcaagcctgatattaatattgctgcagaaattatgcccttctctttgcttgagaaatggctccttgatgaggcaggttgccaagagaaattagttggttgttgtggtgatgccaagtttttctaa

SEQ ID NO: 69 - protein, 336 aa, Citrus clementina
MGRPPCCDKIGIKKGPWTPEEDIILVSYIQEHGPGNWRAVPTNTGLLRCSKSCRLRWTNYLRPGIKRGNFTDQEEKMIIHLQALLGNRWAAIASYLPQRTDNDIKNYWNTHLKKKVRKLQLAAAGCSEDNSQYRDELASASSQQISRGQWERRLQTDIHMAKQALCAALSPDKASILSELKPANGFISYTKPAVQAPAYASSTENIAKLLKGWTRNAQKSASSNSGVTDQNSINNNVNHIAGAESASSEETPSKVASNSTGIELSEAFESLFGFESFDSSNSTDLSQSVTPESSTFQDYESKQLLLDPSAGADDDQMPQLSLLEKWLFDDQGGKDI

SEQ ID NO: 70 - DNA, 1011 bp, Citrus clementina
atggggaggccaccttgttgtgacaaaattggtatcaagaaagggccatggactccagaagaagatatcattttagtttcttatattcaagagcatggccctggaaattggagggctgttcccactaatacaggattgcttagatgcagcaaaagttgcaggcttagatggactaattacctaaggccagggatcaagcgtgggaatttcactgatcaagaagagaagatgataattcatctgcaagcacttttgggcaacagatgggcggctattgcttcttatctccctcagagaactgacaatgacatcaagaactattggaatactcacttgaagaagaaggtgaggaagctgcaactagctgctgctggctgctctgaagataatagccaatatagagatgagctagcttcagcttcttcacagcaaatctcaaggggtcagtgggagagaaggctgcagactgatattcacatggctaagcaagctctatgtgcggccttgtcaccagataaagcgagtattttgtctgaattgaagcctgctaatgggttcatttcctacacaaaaccagcagttcaagcaccagcttacgcttcaagcactgagaacattgctaagttgctcaaagggtggaccagaaacgctcaaaaaagtgcttcttcgaactcaggtgttactgatcagaattcaattaataacaatgttaatcacattgctggggcagaatctgcttctagtgaagagactccaagcaaagttgcaagcaacagtactggcatagaattatcagaggcttttgaatcgttgtttggttttgagtcttttgattcgtcaaattctaccgatttatctcaatctgtgacccctgagtctagcacttttcaagattatgagagcaagcaattgttattagatcctagtgctggtgctgatgatgatcaaatgccacagctgtcattgcttgagaagtggctttttgatgatcaaggagggaaagatatctaa

SEQ ID NO: 71 - protein, 265 aa, Oryza sativa
MSPAEPTREESVYKAKLAEQAERYEEMVEYMERVARAAGGASGGEELTVEERNLLSVAYKNVIGARRASWRIISSIEQKEEGRGNDAHAATIRSYRGKIEAELARICDGILALLDSHLVPSAGAAESKVFYLKMKGDYHRYLAEFKSGDERKQAAESTMNAYKAAQDIALADLAPTHPIRLGLALNFSVFYYEILNSPDRACNLAKQAFDEAISELDSLGEESYKDSTLIMQLLRDNLTLWTSDANDDGGDEIKEAAAPKEPGDQ

SEQ ID NO: 72 - DNA, 798 bp, Oryza sativa
atgtcgccggcggagccgacgagggaggagagcgtgtacaaggcgaagctggcggagcaggcggagcggtacgaggagatggtggagtacatggagcgcgtggcgcgcgcggcggggggcgcctccggcggggaggagctcacggtggaggagcggaacctgctgtccgtggcgtacaagaacgtcatcggcgcccgccgcgcgtcgtggcggatcatctcgtcgatcgagcagaaggaggagggccgcgggaacgacgcccacgccgccaccatccgctcctacaggggcaagatcgaggccgagctcgcccgcatctgcgacggcatcctggccctgctcgactcccacctcgtcccctccgccggcgccgccgagtccaaggtcttctacctcaagatgaagggcgactaccacaggtaccttgcggagtttaagtctggcgacgagaggaagcaggctgcggagagcaccatgaatgcatacaaggctgctcaggacattgctctcgcagatttggctccgacccaccccataaggcttgggcttgcactcaacttttcagtgttctactatgagatcttgaactcccctgaccgtgcctgcaacctcgcgaagcaggcgtttgatgaggccatatcagaactggacagccttggtgaagaatcctacaaggacagcactttgatcatgcagctcctgcgtgacaacttgactctgtggacttcagatgccaatgatgatggtggtgacgaaatcaaggaagccgcagctccaaaagagcctggggatcagtga

SEQ ID NO: 73 - protein, 153 aa, Oryza sativa
MRFFLKLAPRCSVLLLLLLVTASRGLNIGDLLGSTPAKDQGCSRTCESQFCTIAPLLRYGKYCGILYSGCPGERPCDALDACCMVHDHCVDTHNDDYLNTMCNENLLSCIDRVSGATFPGNKCNVGQTASVIRGVIETAVFAGKILHKRDDGQ

SEQ ID NO: 74 - DNA, 462 bp, Oryza sativa
atgaggttcttcctcaagctcgctcctcggtgttccgtgctgcttctcctcttgctggtgacggcgtcgcgggggcttaacatcggcgacctgcttggcagcacgccggcgaaggaccagggatgtagccggacgtgcgaatcccagttttgcacaattgcacctctgctgaggtacggcaagtactgcgggatcctctacagcgggtgccccggcgagaggccatgcgacgcgctcgacgcctgctgcatggtgcacgaccactgcgtcgacacccacaacgacgactacctgaacacgatgtgcaacgagaacctgctgagctgcatcgaccgggtgagcggggcgacgttcccggggaacaagtgcaacgtcggccagacggcgtccgtcatcaggggggtcatcgagacggccgtgttcgccggcaagatcctccacaagcgcgacgacggccaatag

SEQ ID NO: 75 - protein, 163 aa, Oryza sativa
MARGGSFSRLRLRAGVVVAAAAAALLLFAVVAPPAAALNIGLQSAGDGASKAGLCSRTCESDHCTTPPLLRYGKYCGILYSGCPGEQPCDELDACCMHHDNCVQAKNDYLSTACNEELLECLARLREGSSTFQGNKCMIDEVIDVISLVIEAAVVAGRLLHKP

SEQ ID NO: 76 - DNA, 492 bp, Oryza sativa
atggcgcgcggcgggagcttctcgcggctgcggctgcgcgcgggggtcgttgtcgccgccgccgccgccgccctgctcctcttcgccgtcgtcgcgccgcccgccgcggcgctcaacatcggcctccagtccgccggcgacggcgcgagcaaggccgggttgtgcagccgcacgtgcgagtccgaccactgcacgacgccgccgttgctgcgctacggcaagtactgcggcatcctgtacagcggctgccccggcgagcagccgtgcgacgagctcgacgcctgctgcatgcaccacgacaactgcgtccaggccaagaatgactacctgagcacggcgtgcaacgaggagttgctggagtgcctggcgaggctgcgggagggctcgtcgacgttccaggggaacaagtgcatgatcgacgaggtcatcgacgtgatctcgctcgtcatcgaggccgccgtcgtcgccggcaggctgctgcacaagccttag

SEQ ID NO: 77 - protein, 167 aa, Sorghum bicolor
MDGRRRELAVGRHPLQRRCSRRRLLAPLLILLLAVASSQSPTAAGSIFGGGDDDSDCSRECESQHCTAPLMRYGKYCGVSYTGCPGEVPCDAIDACCMLHDACVQATDNDYLNLLCNQSLLDCVAAARPAAAAATFQGNRCNVTDVADEITTVVEAAVYARGILHKP

SEQ ID NO: 78 - DNA, 504 bp, Sorghum bicolor
atggacggaagaagaagggagctcgccgtcggccgtcatcccctgcaacggcggtgtagtcgtcgtcgtcttcttgctcctcttctcatcctgctgctcgccgtcgccagcagccagtcacccaccgccgccggcagcatcttcggtggcggcgacgacgactcggattgcagccgagagtgcgagtcccagcactgcacggcgccgctgatgcgctacggcaagtactgcggcgtgtcctacacggggtgccccggcgaggtcccctgcgacgccatcgacgcctgctgcatgctccacgacgcctgcgtccaggccaccgacaacgactacctcaacttgctgtgcaaccagagcctgctggactgcgtggcggcggcgaggccggcggcggcggcggccacgttccaggggaaccggtgcaacgtcacggacgtcgccgacgagatcaccaccgtcgtggaggccgccgtgtacgccaggggcatcctgcacaagccctag

SEQ ID NO: 79 - protein, 154 aa, Sorghum bicolor
MASVLAFSRCSSLLLLLLATASQALNVGDLLGTAPSGSKDCSRTCESSFCIVPPLLRYGKYCGILYSGCPGEKPCDALDACCMVHDHCVATHNNDYLNTRCNENLLSCLDRVSPAGPTFPGNECGVGQTASVIRGVIESAVLAGKILHKRDDGP

SEQ ID NO: 80 - DNA, 465 bp, Sorghum bicolor
atggcatccgttctcgccttctcccggtgttcgtcgctgcttctcctcctgctggcgacggcgtcacaggccctcaacgtcggcgacctgctcgggacagcgccttcggggagcaaggattgtagccggacgtgcgaatcatcgttctgcatagtcccgccgctgctgaggtacgggaagtactgcgggatcctgtacagcggctgccccggcgagaagccctgcgacgccctcgacgcctgctgcatggtccacgaccactgcgtcgccacccacaacaatgactacctgaacacgcggtgcaacgagaacctgctgagctgcctcgacagggtgagcccagcggggccgacgttcccggggaacgagtgcggcgtcggccagacggcgtccgtcatacgtggggtcatcgagtcggcagtgctcgcgggcaagatccttcacaagcgcgacgacggcccgtag

SEQ ID NO: 81 - protein, 155 aa, Sorghum bicolor
MERGSSWRRLTVVVGILVCAAVFSPPAAALNIGIQSAGDGASKQQACSRTCESDHCTTAPFLRYGKYCGILYSGCPGEQPCDALDACCMHHDNCVQAKKDYLSTSCNEALLECLARLREGTSTFDGNKCMIDEVIDVISVVIEAAVVAGRVLHKP

SEQ ID NO: 82 - DNA, 468 bp, Sorghum bicolor
atggagcgcggcagttcctggcggcggctcaccgtggtcgtcggcatccttgtctgcgcggccgtcttctcgccgcccgccgccgcgctcaacatcggcatccagtccgccggcgacggcgcgagcaagcagcaggcgtgcagccgcacatgcgagtcggaccactgcacgacggcgccgttcctgcggtacggcaagtactgcggcatcctgtacagcggctgccccggcgagcagccgtgcgacgcgctggacgcctgctgcatgcaccacgacaactgcgtccaggcaaagaaggactacctgagcacgtcttgcaacgaggcgctgctggaatgcctggcgaggctgcgggagggcacgtccacgttcgacgggaacaagtgcatgatcgacgaggtcatcgacgtgatctccgtcgtcatagaggccgccgtcgtcgccggcagggtgctgcacaagccgtag

SEQ ID NO: 83 - protein, 152 aa, Vitis vinifera
MKLAMTLLLCSLIGLIFSATPTLALNIGVQATDGSVTLSKECSRKCESEFCSVPPFLRYGKYCGLLYSGCPGEKPCDGLDACCMKHDACVQAKNNDYLSQECSQNFINCMNSFKSSGGHTFKGNKCQVDEVIDVITLVMEAALLAGRYLHKP

SEQ ID NO: 84 - DNA, 459 bp, Vitis vinifera
atgaagttagctatgactctcttgttgtgttccctcattggccttatcttttctgccactcccacccttgctctcaacattggtgttcaagccacagatggctccgtcactctgagtaaagaatgcagtagaaaatgtgaatctgaattctgttcagtgcctccatttctgagatatggcaagtattgtggactcctgtatagtgggtgccctggggagaagccatgtgatggcctggatgcttgttgcatgaagcatgatgcctgtgtacaagccaaaaacaatgactatctgagccaagagtgcagccaaaacttcataaactgcatgaacagcttcaagagctcaggagggcatacattcaagggcaacaaatgccaagtagatgaagttattgatgtcatcacccttgtcatggaggctgctttgcttgctggaagataccttcataagccttag

SEQ ID NO: 85 - protein, 620 aa, Arabidopsis thaliana
MTSSKMEQRSLLCFLYLLLLFNFTLRVAGNAEGDALTQLKNSLSSGDPANNVLQSWDATLVTPCTWFHVTCNPENKVTRVDLGNAKLSGKLVPELGQLLNLQYLELYSNNITGEIPEELGDLVELVSLDLYANSISGPIPSSLGKLGKLRFLRLNNNSLSGEIPMTLTSVQLQVLDISNNRLSGDIPVNGSFSLFTPISFANNSLTDLPEPPPTSTSPTPPPPSGGQMTAAIAGGVAAGAALLFAVPAIAFAWWLRRKPQDHFFDVPAEEDPEVHLGQLKRFTLRELLVATDNFSNKNVLGRGGFGKVYKGRLADGNLVAVKRLKEERTKGGELQFQTEVEMISMAVHRNLLRLRGFCMTPTERLLVYPYMANGSVASCLRERPEGNPALDWPKRKHIALGSARGLAYLHDHCDQKIIHRDVKAANILLDEEFEAVVGDFGLAKLMNYNDSHVTTAVRGTIGHIAPEYLSTGKSSEKTDVFGYGVMLLELITGQKAFDLARLANDDDIMLLDWVKEVLKEKKLESLVDAELEGKYVETEVEQLIQMALLCTQSSAMERPKMSEVVRMLEGDGLAERWEEWQKEEMPIHDFNYQAYPHAGTDWLIPYSNSLIENDYPSGPR

SEQ ID NO: 86 - DNA, 1863 bp, Arabidopsis thaliana
atgacaagttcaaaaatggaacaaagatcactcctttgcttcctttatctgctcctactattcaatttcactctcagagtcgctggaaacgctgaaggtgatgctttgactcagctgaaaaacagtttgtcatcaggtgaccctgcaaacaatgtactccaaagctgggatgctactcttgttactccatgtacttggtttcatgttacttgcaatcctgagaataaagttactcgtgttgaccttgggaatgcaaaactatctggaaagttggttccagaacttggtcagcttttaaacttgcagtacttggagctttatagcaataacattacaggggagatacctgaggagcttggcgacttggtggaactagtaagcttggatctttacgcaaacagcataagcggtcccatcccttcgtctcttggcaaactaggaaaactccggttcttgcgtcttaacaacaatagcttatcaggggaaattccaatgactttgacttctgtgcagctgcaagttctggatatctcaaacaatcggctcagtggagatattcctgttaatggttctttttcgctcttcactcctatcagttttgcgaataatagcttaacggatcttcccgaacctccgcctacttctacctctcctacgccaccaccaccttcaggggggcaaatgactgcagcaatagcagggggagttgctgcaggtgcagcacttctatttgctgttccagccattgcgtttgcttggtggctcagaagaaaaccacaggaccacttttttgatgtacctgctgaagaagacccagaggttcatttaggacaactcaaaaggtttaccttgcgtgaactgttagttgctactgataactttagcaataaaaatgtattgggtagaggtggttttggtaaagtgtataaaggacgtttagccgatggcaatctagtggctgtcaaaaggctaaaagaagaacgtaccaagggtggggaactgcagtttcaaaccgaagttgagatgatcagtatggccgttcataggaacttgcttcggcttcgtggcttttgcatgactccaactgaaagattacttgtttatccctacatggctaatggaagtgttgcttcttgtttaagagagcgtcctgaaggcaatccagcacttgattggccaaaaagaaagcatattgctctgggatcagcaagggggcttgcgtatttacatgatcattgcgaccaaaaaatcattcaccgggatgttaaagctgctaatatattgttagatgaagagtttgaagctgttgttggagattttgggctcgcaaaattaatgaattataatgactcccatgtgacaactgctgtacgcggtacaattggccatatagcgcccgagtacctctcgacaggaaaatcttctgagaagactgatgtttttgggtacggggtcatgcttctcgagctcatcactggacaaaaggctttcgatcttgctcggcttgcaaatgatgatgatatcatgttactcgactgggtgaaagaggttttgaaagagaagaagttggaaagccttgtggatgcagaactcgaaggaaagtacgtggaaacagaagtggagcagctgatacaaatggctctgctctgcactcaaagttctgcaatggaacgtccaaagatgtcagaagtagtgagaatgctggaaggagatggtttagctgagagatgggaagaatggcaaaaggaggagatgccaatacatgattttaactatcaagcctatcctcatgctggcactgactggctcatcccctattccaattcccttatcgaaaacgattacccctcgggtccaagataa

SEQ ID NO: 87 - protein, 601 aa, Arabidopsis thaliana
MEHGSSRGFIWLILFLDFVSRVTGKTQVDALIALRSSLSSGDHTNNILQSWNATHVTPCSWFHVTCNTENSVTRLDLGSANLSGELVPQLAQLPNLQYLELFNNNITGEIPEELGDLMELVSLDLFANNISGPIPSSLGKLGKLRFLRLYNNSLSGEIPRSLTALPLDVLDISNNRLSGDIPVNGSFSQFTSMSFANNKLRPRPASPSPSPSGTSAAIVVGVAAGAALLFALAWWLRRKLQGHFLDVPAEEDPEVYLGQFKRFSLRELLVATEKFSKRNVLGKGRFGILYKGRLADDTLVAVKRLNEERTKGGELQFQTEVEMISMAVHRNLLRLRGFCMTPTERLLVYPYMANGSVASCLRERPEGNPALDWPKRKHIALGSARGLAYLHDHCDQKIIHLDVKAANILLDEEFEAVVGDFGLAKLMNYNDSHVTTAVRGTIGHIAPEYLSTGKSSEKTDVFGYGVMLLELITGQKAFDLARLANDDDIMLLDWVKEVLKEKKLESLVDAELEGKYVETEVEQLIQMALLCTQSSAMERPKMSEVVRMLEGDGLAERWEEWQKEEMPIHDFNYQAYPHAGTDWLIPYSNSLIENDYPSGPR

SEQ ID NO: 88 - DNA, 1806 bp, Arabidopsis thaliana
atggaacatggatcatcccgtggctttatttggctgattctatttctcgattttgtttccagagtcaccggaaaaacacaagttgatgctctcattgctctaagaagcagtttatcatcaggtgaccatacaaacaatatactccaaagctggaatgccactcacgttactccatgttcatggtttcatgttacttgcaatactgaaaacagtgttactcgtcttgacctggggagtgctaatctatctggagaactggtgccacagcttgctcagcttccaaatttgcagtacttggaactttttaacaataatattactggggagatacctgaggagcttggcgacttgatggaactagtaagcttggacctttttgcaaacaacataagcggtcccatcccttcctctcttggcaaactaggaaaactccgcttcttgcgtctttataacaacagcttatctggagaaattccaaggtctttgactgctctgccgctggatgttcttgatatctcaaacaatcggctcagtggagatattcctgttaatggttccttttcgcagttcacttctatgagttttgccaataataaattaaggccgcgacctgcatctccttcaccatcaccttcaggaacgtctgcagcaatagtagtgggagttgctgcgggtgcagcacttctatttgcgcttgcttggtggctgagaagaaaactgcagggtcactttcttgatgtacctgctgaagaagacccagaggtttatttaggacaatttaaaaggttctccttgcgtgaactgctagttgctacagagaaatttagcaaaagaaatgtattgggcaaaggacgttttggtatattgtataaaggacgtttagctgatgacactctagtggctgtgaaacggctaaatgaagaacgtaccaagggtggggaactgcagtttcaaaccgaagttgagatgatcagtatggccgttcataggaacttgcttcggcttcgtggcttttgcatgactccaactgaaagattacttgtttatccctacatggctaatggaagtgttgcttcttgtttaagagagcgtcctgaaggcaatccagcccttgactggccaaaaagaaagcatattgctctgggatcagcaagggggctcgcatatttacacgatcattgcgaccaaaagatcattcacctggatgtgaaagctgcaaatatactgttagatgaagagtttgaagctgttgttggagattttgggctagcaaaattaatgaattataacgactcccatgtgacaactgctgtacggggtacgattggccatatagcgcccgagtacctctcgacaggaaaatcttctgagaagactgatgtttttgggtacggggtcatgcttctcgagctcatcactggacaaaaggctttcgatcttgctcggcttgcaaatgatgatgatatcatgttactcgactgggtgaaagaggttttgaaagagaagaagttggaaagccttgtggatgcagaactcgaaggaaagtacgtggaaacagaagtggagcagctgatacaaatggctctgctctgcactcaaagttctgcaatggaacgtccaaagatgtcagaagtagtgagaatgctggaaggagatggtttagctgagagatgggaagaatggcaaaaggaggagatgccaatacatgattttaactatcaagcctatcctcatgctggcactgactggctcatcccctattccaattcccttatcgaaaacgattacccctcggggccaagataa

SEQ ID NO: 89 - protein, 628 aa, Arabidopsis thaliana
MGRKKFEAFGFVCLISLLLLFNSLWLASSNMEGDALHSLRANLVDPNNVLQSWDPTLVNPCTWFHVTCNNENSVIRVDLGNADLSGQLVPQLGQLKNLQYLELYSNNITGPVPSDLGNLTNLVSLDLYLNSFTGPIPDSLGKLFKLRFLRLNNNSLTGPIPMSLTNIMTLQVLDLSNNRLSGSVPDNGSFSLFTPISFANNLDLCGPVTSRPCPGSPPFSPPPPFIPPPIVPTPGGYSATGAIAGGVAAGAALLFAAPALAFAWWRRRKPQEFFFDVPAEEDPEVHLGQLKRFSLRELQVATDSFSNKNILGRGGFGKVYKGRLADGTLVAVKRLKEERTPGGELQFQTEVEMISMAVHRNLLRLRGFCMTPTERLLVYPYMANGSVASCLRERPPSQLPLAWSIRQQIALGSARGLSYLHDHCDPKIIHRDVKAANILLDEEFEAVVGDFGLARLMDYKDTHVTTAVRGTIGHIAPEYLSTGKSSEKTDVFGYGIMLLELITGQRAFDLARLANDDDVMLLDWVKGLLKEKKLEMLVDPDLQSNYTEAEVEQLIQVALLCTQSSPMERPKMSEVVRMLEGDGLAEKWDEWQKVEVLRQEVELSSHPTSDWILDSTDNLHAMELSGPR

SEQ ID NO: 90 - DNA, 1887 bp, Arabidopsis thaliana
atggggagaaaaaagtttgaagcttttggttttgtctgcttaatctcactgcttcttctgtttaattcgttatggcttgcctcttctaacatggaaggtgatgcactgcacagtttgagagctaatctagttgatccaaataatgtcttgcaaagctgggatcctacgcttgttaatccgtgtacttggtttcacgtaacgtgtaacaacgagaacagtgttataagagtcgatcttgggaatgcagacttgtctggtcagttggttcctcagctaggtcagctcaagaacttgcagtacttggagctttatagtaataacataaccgggccggttccaagcgatcttgggaatctgacaaacttagtgagcttggatctttacttgaacagcttcactggtccaattccagattctctaggaaagctattcaagcttcgctttcttcggctcaacaataacagtctcaccggaccaattcccatgtcattgactaatatcatgacccttcaagttttggatctgtcgaacaaccgattatccggatctgttcctgataatggttccttctcgctcttcactcccatcagttttgctaacaacttggatctatgcggcccagttactagccgtccttgtcctggatctcccccgttttctcctccaccaccttttataccacctcccatagttcctacaccaggtgggtatagtgctactggagccattgcgggaggagttgctgctggtgctgctttactatttgctgcccctgctttagcttttgcttggtggcgtagaagaaaacctcaagaattcttctttgatgttcctgccgaagaggaccctgaggttcacttggggcagcttaagcggttctctctacgggaacttcaagtagcaactgatagcttcagcaacaagaacattttgggccgaggtgggttcggaaaagtctacaaaggccgtcttgctgatggaacacttgttgcagtcaaacggcttaaagaagagcgaaccccaggtggcgagctccagtttcagacagaagtggagatgataagcatggccgttcacagaaatctcctcaggctacgcggtttctgtatgacccctaccgagagattgcttgtttatccttacatggctaatggaagtgtcgcttcctgtttgagagaacgtccaccatcacagttgcctctagcctggtcaataagacagcaaatcgcgctaggatcagcgaggggtttgtcttatcttcatgatcattgcgaccccaaaattattcaccgtgatgtgaaagctgctaatattctgttggacgaggaatttgaggcggtggtaggtgatttcgggttagctagacttatggactataaagatactcatgtcacaacggctgtgcgtgggactattggacacattgctcctgagtatctctcaactggaaaatcttcagagaaaactgatgtttttggctacgggatcatgcttttggaactgattacaggtcagagagcttttgatcttgcaagactggcgaatgacgatgacgttatgctcctagattgggtgaaagggcttttgaaggagaagaagctggagatgcttgtggatcctgacctgcaaagcaattacacagaagcagaagtagaacagctcatacaagtggctcttctctgcacacagagctcacctatggaacgacctaagatgtctgaggttgttcgaatgcttgaaggtgacggtttagcggagaaatgggacgagtggcagaaagtggaagttctcaggcaagaagtggagctctcttctcaccccacctctgactggatccttgattcgactgataatcttcatgctatggagttgtctggtccaagataa

SEQ ID NO: 91 - protein, 625 aa, Arabidopsis thaliana
MESSYVVFILLSLILLPNHSLWLASANLEGDALHTLRVTLVDPNNVLQSWDPTLVNPCTWFHVTCNNENSVIRVDLGNAELSGHLVPELGVLKNLQYLELYSNNITGPIPSNLGNLTNLVSLDLYLNSFSGPIPESLGKLSKLRFLRLNNNSLTGSIPMSLTNITTLQVLDLSNNRLSGSVPDNGSFSLFTPISFANNLDLCGPVTSHPCPGSPPFSPPPPFIQPPPVSTPSGYGITGAIAGGVAAGAALLFAAPAIAFAWWRRRKPLDIFFDVPAEEDPEVHLGQLKRFSLRELQVASDGFSNKNILGRGGFGKVYKGRLADGTLVAVKRLKEERTPGGELQFQTEVEMISMAVHRNLLRLRGFCMTPTERLLVYPYMANGSVASCLRERPPSQPPLDWPTRKRIALGSARGLSYLHDHCDPKIIHRDVKAANILLDEEFEAVVGDFGLAKLMDYKDTHVTTAVRGTIGHIAPEYLSTGKSSEKTDVFGYGIMLLELITGQRAFDLARLANDDDVMLLDWVKGLLKEKKLEMLVDPDLQTNYEERELEQVIQVALLCTQGSPMERPKMSEVVRMLEGDGLAEKWDEWQKVEILREEIDLSPNPNSDWILDSTYNLHAVELSGPR

SEQ ID NO: 92 - DNA, 1878 bp, Arabidopsis thaliana
atggagtcgagttatgtggtgtttatcttactttcactgatcttacttccgaatcattcactgtggcttgcttctgctaatttggaaggtgatgctttgcatactttgagggttactctagttgatccaaacaatgtcttgcagagctgggatcctacgctagtgaatccttgcacatggttccatgtcacttgcaacaacgagaacagtgtcataagagttgatttggggaatgcagagttatctggccatttagttccagagcttggtgtgctcaagaatttgcagtatttggagctttacagtaacaacataactggcccgattcctagtaatcttggaaatctgacaaacttagtgagtttggatctttacttaaacagcttctccggtcctattccggaatcattgggaaagctttcaaagctgagatttctccggcttaacaacaacagtctcactgggtcaattcctatgtcactgaccaatattactacccttcaagtgttagatctatcaaataacagactctctggttcagttcctgacaatggctccttctcactcttcacacccatcagttttgctaataacttagacctatgtggacctgttacaagtcacccatgtcctggatctcccccgttttctcctccaccaccttttattcaacctcccccagtttccaccccgagtgggtatggtataactggagcaatagctggtggagttgctgcaggtgctgctttgctctttgctgctcctgcaatagcctttgcttggtggcgacgaagaaagccactagatattttcttcgatgtccctgccgaagaagatccagaagttcatctgggacagctcaagaggttttctttgcgggagctacaagtggcgagtgatgggtttagtaacaagaacattttgggcagaggtgggtttgggaaagtctacaagggacgcttggcagacggaactcttgttgctgtcaagagactgaaggaagagcgaactccaggtggagagctccagtttcaaacagaagtagagatgataagtatggcagttcatcgaaacctgttgagattacgaggtttctgtatgacaccgaccgagagattgcttgtgtatccttacatggccaatggaagtgttgcttcgtgtctcagagagaggccaccgtcacaacctccgcttgattggccaacgcggaagagaatcgcgctaggctcagctcgaggtttgtcttacctacatgatcactgcgatccgaagatcattcaccgtgacgtaaaagcagcaaacatcctcttagacgaagaattcgaagcggttgttggagatttcgggttggcaaagctaatggactataaagacactcacgtgacaacagcagtccgtggcaccatcggtcacatcgctccagaatatctctcaaccggaaaatcttcagagaaaaccgacgttttcggatacggaatcatgcttctagaactaatcacaggacaaagagctttcgatctcgctcggctagctaacgacgacgacgtcatgttacttgactgggtgaaaggattgttgaaggagaagaagctagagatgttagtggatccagatcttcaaacaaactacgaggagagagaactggaacaagtgatacaagtggcgttgctatgcacgcaaggatcaccaatggaaagaccaaagatgtctgaagttgtaaggatgctggaaggagatgggcttgcggagaaatgggacgaatggcaaaaagttgagattttgagggaagagattgatttgagtcctaatcctaactctgattggattcttgattctacttacaatttgcacgccgttgagttatctggtccaaggtaa

SEQ ID NO: 93 - protein, 628 aa, Oryza sativa
MAEARLLRRRRLCLAVPFVWVVAVAVSRVGANTEGDALYSLRQSLKDANNVLQSWDPTLVNPCTWFHVTCNPDNSVIRVDLGNAQLSGALVPQLGQLKNLQYLELYSNNISGTIPNELGNLTNLVSLDLYLNNFTGFIPETLGQLYKLRFLRLNNNSLSGSIPKSLTNITTLQVLDLSNNNLSGEVPSTGSFSLFTPISFANNKDLCGPGTTKPCPGAPPFSPPPPFNPPTPTVSQGDSKTGAIAGGVAAAAALLFAVPAIGFAWWRRRKPEEHFFDVPAEEDPEVHLGQLKRFSLRELQVATDNFSNKNILGRGGFGKVYKGRLADGSLVAVKRLKEERTPGGELQFQTEVEMISMAVHRNLLRLRGFCMTPTERLLVYPYMANGSVASRLRERQPNDPPLEWQTRTRIALGSARGLSYLHDHCDPKIIHRDVKAANILLDEDFEAVVGDFGLAKLMDYKDTHVTTAVRGTIGHIAPEYLSTGKSSEKTDVFGYGIMLLELITGQRAFDLARLANDDDVMLLDWVKGLLKEKKVEMLVDPDLQSGFVEHEVESLIQVALLCTQGSPMDRPKMSEVVRMLEGDGLAERWEEWQKVEVVRQEAELAPRHNDWIVDSTYNLRAMELSGPR

SEQ ID NO: 94 - DNA, 1887 bp, Oryza sativa
atggcggaggcgcggctgctgcggcggcggcggctgtgcttggcggtgccgttcgtgtgggtggtggcggtggccgtgagccgggtcggcgccaacacggagggtgatgccctatatagtctgcgccaaagtctgaaagatgctaacaatgtgctgcagagttgggatcccactctggtcaatccatgcacatggttccatgtaacttgtaaccctgacaacagcgtgatcagagttgatcttggaaatgcacaactgtcaggtgcattggttccccagcttgggcagttgaaaaatctgcaatatctggagctttacagcaacaacataagtgggacaatacctaatgaactgggaaacttaactaacttggtcagtttggatctttacctgaacaacttcactggttttattccggaaaccttggggcaactctacaagctgcgtttccttcgtcttaacaacaacagtctttctggttcaattccaaaatccttgaccaatatcactactcttcaagttctggatctctcaaataacaatctctcaggagaggttccgtctactggctccttttcactctttacccctataagttttgctaataataaagatctttgtggcccgggtactacaaaaccctgccctggagctccacctttttctccaccacctcctttcaatcccccaacacctactgtgtcacaaggtgactccaaaactggagcaattgctggaggtgttgctgcagctgctgcattgctgtttgcggttccggcaattggatttgcatggtggcggcggcgtaaacctgaagaacacttctttgatgtccctgctgaggaggatccggaagtgcaccttggccaacttaagagattctcactccgggagcttcaagttgctactgataactttagcaataagaatattctgggaagaggtggctttggaaaggtgtacaaaggtagactggcagatggctcgttggtagcagtgaaaagattaaaagaagaacgtacccctggtggtgagctccagttccaaacagaagttgaaatgattagcatggcggtgcataggaacctgcttcggctccgtggattttgcatgacgcctacagaacggttacttgtctatccctacatggctaatgggagtgtcgcatcacgattgcgagagcggcagccaaatgatccgccgcttgaatggcaaacaagaactcggattgcgctgggatctgccagaggattgtcctacttgcacgaccattgtgatcccaagatcattcatcgtgatgtcaaagctgcaaatattctgttggatgaagattttgaggcagtcgtgggtgactttggactggccaaacttatggattacaaggacactcatgtaaccacagctgttcgtgggacgatcggacacattgctcctgagtacctctctactgggaagtcctctgagaagactgatgtttttggctatggaatcatgcttcttgagctcattacaggacaaagggcatttgatcttgctcgtcttgcaaacgatgatgatgtgatgttgctcgattgggtgaaagggctcctgaaagagaagaaggtggagatgctggtggacccggacctccagagcggcttcgtggagcatgaggtggagtcactcatccaggtggctctgctctgcacgcagggctccccgatggaccggcccaagatgtcggaggtggtgaggatgctggagggcgatggcctcgcggagcggtgggaggagtggcagaaggtggaggtggtccggcaggaggcggagctggccccccgccacaacgactggatcgtcgactcgacctacaatctccgggcaatggagctgtccggcccgaggtaa

SEQ ID NO: 95 - protein, 624 aa, Oryza sativa
MAAHRWAVWAVLLLRLLVPAARVLANMEGDALHSLRTNLVDPNNVLQSWDPTLVNPCTWFHVTCNNDNSVIRVDLGNAALSGTLVPQLGQLKNLQYLELYSNNISGTIPSELGNLTNLVSLDLYLNNFTGPIPDSLGNLLKLRFLRLNNNSLSGSIPKSLTAITALQVLDLSNNNLSGEVPSTGSFSLFTPISFANNPSLCGPGTTKPCPGAPPFSPPPPYNPPTPVQSPGSSSSTGAIAGGVAAGAALLFAIPAIGFAWYRRRKPQEHFFDVPAEEDPEVHLGQLKRFSLRELQVATDTFSNKNILGRGGFGKVYKGRLADGSLVAVKRLKEERTPGGELQFQTEVEMISMAVHRNLLRLRGFCMTPTERLLVYPYMANGSVASRLRERPPSEPPLDWRTRRRIALGSARGLSYLHDHCDPKIIHRDVKAANILLDEDFEAVVGDFGLAKLMDYKDTHVTTAVRGTIGHIAPEYLSTGKSSEKTDVFGYGIMLLELITGQRAFDLARLANDDDVMLLDWVKGLLKEKRLEMLVDPDLQSNYIDVEVESLIQVALLCTQGSPTERPKMAEVVRMLEGDGLAERWEEWQKIEVVRQEVELGPHRNSEWIVDSTDNLHAVELSGPR

SEQ ID NO: 96 - DNA, 1875 bp, Oryza sativa
atggcggcgcatcggtgggcggtgtgggcggtgctgctgctgcggctgctcgtgccggcggcgcgggtgctcgccaacatggaaggtgatgcattgcatagcttgaggactaatttagttgatcctaataatgttctacaaagttgggacccaactctggtcaatccgtgcacttggtttcatgttacttgcaataacgacaacagtgttatcagagttgatcttgggaatgctgcactatcaggcactttggtcccacaacttgggcaactaaaaaacttgcaatacctggagctctacagtaataacataagcggaacgatacctagtgaacttggaaacctcacaaacttggtcagtttggatttgtacttgaacaacttcactggtccaataccagattcacttggaaacctattgaagctacgattcctgcgtcttaacaataacagcctttcgggttcaattcctaaatcactaactgctatcactgccctacaagttctagatctttcaaacaacaatttgtctggagaagttccatcaactggttccttttcattattcacccctatcagttttgccaacaacccttccttgtgtggtcctgggaccacaaaaccttgccctggtgctccccccttttccccacctcctccatataatcctccaactcctgtgcagtcaccagggagttcatctagtactggagcaattgctggtggagtggctgctggagcagccttgctatttgctattcctgctattggttttgcatggtatcggcgcaggaaaccccaagagcatttctttgatgtgcctgctgaggaggatccagaggtccatcttggccagcttaaaagattttcactacgagaactacaagttgcaacagataccttcagcaataaaaacattctcggaagaggtgggtttggcaaggtctataaaggaagattagcagatggttctttagtagctgttaagagactaaaggaggagagaacacctggtggggaactacagtttcaaacagaagttgagatgattagcatggctgtacatagaaatctgctgcgtttacgagggttctgtatgacacccacagaaaggttgcttgtgtatccatacatggctaatggaagcgttgcgtcacgtcttagagaacggccaccatcggaacctccacttgattggcgaacaagaagaaggattgcgttgggttccgccagggggctgtcctatttacatgatcattgtgacccaaagattatccatcgtgatgtcaaagctgcaaatattttattagatgaagactttgaagctgtagtaggggactttggtttggccaaactaatggattacaaggatacccatgtaacaactgcagttcgtggaacaattgggcatattgcaccagaatatctttcaacaggaaaatcatctgagaaaactgatgtatttggttatgggattatgcttttggagcttataacaggacaacgtgcctttgaccttgctcgtctagccaatgatgatgatgtcatgctactggactgggtaaaaggattactcaaggagaaaaggctggagatgttggttgatccagatttacagagcaactacattgatgttgaggtagaatcactaatccaggttgctcttctttgcacacaaggctcccccacagaacgccccaagatggcggaggttgtgaggatgcttgaaggtgatggccttgccgagagatgggaggagtggcagaagatagaagtagtacggcaggaggtagagcttggccctcatcggaactcagagtggattgtcgactcgacggacaaccttcatgcggttgagctatcagggccgaggtga

SEQ ID NO: 97 - protein, 530 aa, Sorghum bicolor
MAAAEASRRRRWALWALLLLRLLHPAALVLANTEGDALHSLRTNLNDPNNVLQSWDPTLVNPCTWFHVTCNNDNSVIRVDLGNAALSGTLVPQLGQLKNLQYLELYSNNISGIIPSELGNLTNLVSLDLYLNNFTGSIPDSLGKLLKLRFLRLNNNSLTGSIPKSLTAITALQVLDLSNNNLSGEVPSTGSFSLFTPISFANNPNLCGPGTTKPCPGAPPFSPPPPYNPTTPAQSPGSSSSSTGAIAGGVAAGAALLFAIPAIGFAYWRRRKPQEHFFDVPAEEDPEVHLGQLKRFSLRELQVATDGFSNKNILGRGGFGKVYKGRLADGSLVAVKRLKEERTPGGELQFQTEVEMISMAVHRNLLRLRGFCMTPTERLLVYPYMANGSVASRLRDRPPAEPPLDWQTRRRIALGSARGLSYLHDHCDPKIIHRDVKAANILLDEDFEAVVGDFGLAKLMDYKDTHVTTAVRGTIGHIAPEYLSTGKSSEKTDVFGYGITLLELITGQRAFDLARLANDDDVMLLDWVIN

SEQ ID NO: 98 - DNA, 1593 bp, Sorghum bicolor
atggctgcggcggaggcttcgcggcggcgacggtgggcgttgtgggcgctgctgctgctgcggctgctgcacccggccgcgctcgtgctcgccaacaccgaaggtgatgccttgcatagcttaaggactaacttaaatgatcctaataatgttctacaaagttgggatcccactctggtcaacccctgcacttggtttcatgttacctgcaacaatgacaacagtgttatcagagttgatcttggaaatgctgcactatcaggaactttggttccgcaacttggccagctcaaaaacttgcagtacctggagctctacagtaataatatcagcggcattatacctagtgaacttgggaatcttacaaacttggtcagtttggatttgtacctgaacaacttcactggttcgataccagattcattggggaagctattgaagctgcggttcttgcgtcttaacaacaacagccttactggttcaattccaaaatcattaactgctatcactgcactccaagttctggatctgtcaaataacaatttgtctggagaagttccatcaactggttccttttcattattcacccctatcagttttgcgaacaaccctaatttatgtggtcctggcactacaaaaccttgtcctggtgctcctcccttttctccacctcctccatacaaccctacaacccctgcgcaatcaccaggaagtagctcttccagtactggagcaattgctggtggagtggctgctggcgcagccttgctgtttgctattcctgcaattggttttgcctattggcgacgcaggaaacctcaagagcatttcttcgatgtacctgccgaggaagatccagaggtgcatcttggccagcttaaaagattttcactacgagaattacaagttgcaacagatggcttcagcaataagaacattcttggaagaggtggatttggcaaagtctacaaaggacggctggcagatggatcattagttgctgttaagagactaaaggaagagcgcacgcctggtggggaattacagtttcaaacagaagttgagatgattagtatggctgtacacagaaatctattgcgtcttcgtggattctgtatgacaccaacagaaaggttgcttgtgtatccatacatggctaatggaagtgttgcatcacgtttaagagaccggccaccagctgaacctccgctagattggcaaacaagaagaaggattgcattgggttctgctaggggcctgtcttatttacatgatcattgtgatccaaagattattcatcgtgatgtcaaagctgcaaatattttgttagatgaagacttcgaagctgtggtgggggattttggtttggccaaactaatggattacaaggatacccatgtaactactgctgttcgtggaacgattgggcacattgcacctgaatacctttcaacaggaaaatcctctgagaaaactgatgtatttggctatggaattacgcttttagagcttattacaggacaacgtgcctttgatctagctcgccttgctaatgatgatgatgtcatgcttcttgactgggtaattaactaa

SEQ ID NO: 99 - protein, 622 aa, Sorghum bicolor
MAASLRWWWSAVVLLVVVGVSPVVANTEGDALYSLRQSLKDNNNVLQSWDPTLVNPCTWFHVTCNPDNSVIRLDLGNAQLSGPLVPQLGQLKNMQYLELYSNNISGPIPPELGNLTNLVSLDLYLNNFTGGIPDTLGQLSKLRFLRLNNNSLSGQIPKTLTNINTLQVLDLSNNNLSGGVPSSGSFSLFTPISFANNPNLCGPGTTKPCPGAPPFSPPPPYNPPSPASSKGVSSTGAIAGGVAAGTAFLIAVPAIGYALWRRRKPEEQFFDVPGEEDPEVHLGQLKRFSLRELQVATDNFNNRNVLGRGGFGKVYKGRLSDGSLVAVKRLKEERTPGGELQFQTEVELISMAVHRNLLRLRGFCMTPTERLLVYPYMANGSVASRLRERTENDPPLEWETRARIALGSARGLSYLHDHCDPKIIHRDVKAANILLDEDFEAVVGDFGLAKLMDYKDTHVTTAVRGTIGHIAPEYLSTGKSSEKTDVFGYGIMLLELITGQRAFDLARLANDDDVMLLDWVKALLKEKKLEQLVDPDLQGRYADQEVESLIQVALLCTQGSPMERPKMSEVVRMLEGDGLAERWEQWQKVEVMRQEAELAPRHNDWIVDSTYNLRAVELSGPR

SEQ ID NO: 100 - DNA, 1869 bp, Sorghum bicolor
atggcggcgtcgctgaggtggtggtggtcggcggtggtattgttggtggtggtcggcgtgagcccggttgtcgccaacacggagggtgatgctctttacagcctacggcaaagcctgaaagataacaacaatgtgctgcagagttgggatccaactctggttaatccatgtacatggttccatgttacttgtaaccctgataacagtgtcatcagacttgatcttggaaatgcacaactatcaggtccgttggtgccacagcttgggcaattgaaaaatatgcaatatctagaactttacagtaacaacataagtgggccaataccacctgaactggggaacttaactaacctggtcagtttggatttgtacctcaacaacttcactggaggcattcctgacaccttgggccaactatcaaagttgcggtttctccgtcttaataacaacagtctttctggccaaattccgaaaacgctgaccaatatcaacactctccaagttctggatctctcaaacaacaatctctcaggaggggtgccatcaagtggttcattttctctgtttacacctataagttttgctaacaacccaaatctttgtggccctggtactacaaagccttgtcctggggctcctccattttctccaccccctccatacaatcccccatcaccagcttcatcaaaaggcgtgtccagcactggagcaattgctggaggcgttgcggctggcactgcatttctgattgctgtgcctgctattggatacgcattgtggcggaggcgaaaacctgaagagcaattctttgatgtccctggtgaggaggatcctgaagttcacctaggacaactcaagaggttttcactgagagagcttcaagttgccacagataattttaacaataggaatgtcctaggaagaggtggttttggaaaggtgtacaaagggagactgtcggatggttcactggtagcggtgaagagattaaaggaggaacgcacccctggcggagagctccagttccaaacagaagttgaattgattagcatggcagtgcacaggaatctgcttcggctccgtggattctgcatgactccaacagagcggttgcttgtatatccatacatggctaatgggagtgtcgcgtcgcgccttcgagaacgcacggaaaatgatccccctcttgaatgggaaacaagggccaggatcgcgctgggatcagccagaggtctctcctacttgcacgaccactgtgatcccaagatcattcaccgcgacgtgaaagccgccaacatcctgttggacgaagactttgaagccgtggtgggtgactttggcctcgccaagctcatggattacaaggacacccacgtgacgaccgctgtccgtgggacgatcggccacatcgcccccgagtatctctccaccggaaagtcctctgagaagacggacgtcttcggctatgggatcatgctcctggaactcatcactgggcagagggcgttcgacctcgctcgtctcgcgaatgatgacgacgtcatgctccttgactgggtgaaggcgctgctgaaggagaagaagctggagcagctggtggacccggacctgcagggccggtacgcggaccaggaggtggagtcgctgatccaggtggcgctgctgtgcacacaggggtccccaatggagcggcccaagatgtcagaggtggtgcggatgctggagggcgacgggctggcggagcgttgggagcagtggcagaaggtggaggtgatgcggcaggaggcggagctcgccccgcgccacaacgactggatcgtcgactccacctacaacctcagggccgtcgaactgtccggcccgaggtag

SEQ ID NO: 101 - protein, 626 aa, Sorghum bicolor
MAAAAAAGSWWAVVLAVAVLLGPGRVVANTEGDALYSLRQSLKDANNVLQSWDPTLVNPCTWFHVTCNNDNSVIRVDLGNAQLSGVLVPQLGQLKNLQYLELYSNNISGTIPPELGNLTNLVSLDLYMNNFSGSIPDSLGNLLKLRFLRLNNNSLVGQIPVSLTNISTLQVLDLSNNNLSGQVPSTGSFSLFTPISFANNPGLCGPGTTKPCPGAPPFSPPPPFNPPSPPTQSTGASSTGAIAGGVAAGAALVFAVPAIAFAMWRRRKPEEHFFDVPAEEDPEVHLGQLKKFSLRELQVATDNFSNKNILGRGGFGKVYKGRLADGSLVAVKRLKEERTPGGELQFQTEVEMISMAVHRNLLRLRGFCMTPTERLLVYPYMANGSVASRLRERQQSEPPLKWETRRRIALGSARGLSYLHDHCDPKIIHRDVKAANILLDEDFEAVVGDFGLAKLMDYKDTHVTTAVRGTIGHIAPEYLSTGKSSEKTDVFGYGIMLLELITGQRAFDLARLANDDDVMLLDWVKGLLKEKKVEMLVDPDLQNAYEEIEVENLIQVALLCTQGSPLDRPKMSEVVRMLEGDGLAERWDEWQKVEVVRQEAESAPLRNDWIVDSTYNLRAVELSGPR

SEQ ID NO: 102 - DNA, 1881 bp, Sorghum bicolor
atggcggcggctgcggcggcggggagctggtgggcggtggtccttgcggtggcggtgctgctcgggccgggacgtgtcgtcgccaacaccgagggtgatgctctgtacagcctgcggcagagcttgaaagatgctaacaatgtcttgcagagttgggatcccactcttgttaatccatgtacatggttccacgttacgtgtaacaacgataacagtgttatcagagttgacctcggaaatgcacaattgtctggtgtcctagtgccacaacttggtcagctgaaaaatctccaatatttggagctttacagcaacaacataagtggaacaataccccctgaactggggaacttgactaacttggtcagtttggatctgtatatgaacaacttctctggcagtatccctgacagcctggggaatcttctgaagctgcggttcctacgtcttaacaacaacagcttggttggtcaaattcctgtatccttgaccaatatctccactctccaagtactggatctctcgaacaacaacctctcaggacaagtcccatcaacaggctccttttcactcttcacccctattagttttgccaacaatccaggcctttgtggccctggtactacgaagccctgccctggggctcctcccttttccccgcctcctccattcaatcctccatctcccccaacccaatcaaccggtgcctctagcactggagcaatcgctggaggcgttgctgctggtgcagcattggtgtttgctgttcctgcaattgcatttgcaatgtggcgccgtcgtaaacctgaagagcatttcttcgatgtacctgccgaagaggatccagaagtccatcttggtcagctcaaaaagttttcgttgcgggagcttcaagttgcaactgataatttcagtaacaagaacattttaggaagaggtggttttggaaaagtgtacaagggaaggcttgctgatggctctttggtagcagtgaaaaggctaaaagaggagcgaacacctggtggtgaacttcagttccaaacagaggttgagatgattagcatggcagtgcacaggaaccttctcagacttcgtggtttctgcatgacgcctactgaacggttgctagtctacccatacatggctaatgggagtgtggcatcacgtttacgagagcgacagcaatctgagccacctcttaagtgggaaacaagaagacggattgcgcttggatctgcaagaggactttcttacttgcatgatcactgcgatcccaaaatcatccatcgggatgtcaaagctgcaaatattcttttggatgaggacttcgaggcagttgtgggtgattttgggcttgccaagcttatggactacaaagatacccatgtcacaactgctgtccgtggaacaattggacacattgctcctgagtacctatccactggcaagtcctctgaaaagactgatgtttttggctatgggatcatgcttctggagcttattactggccagagggcatttgatcttgctcgtcttgcaaatgatgacgatgttatgcttcttgactgggtgaaaggactgctgaaggagaagaaggtggagatgctggtggacccagatctgcagaacgcctacgaggagatcgaggtggagaacctgatccaggtggcactcctctgcacgcagggctccccgttggaccgcccaaagatgtcggaggtggtgaggatgctcgaaggtgacggcctggcagagcgctgggacgagtggcagaaagtggaggtggtgaggcaggaggctgagtccgcaccgctccgcaatgactggatcgtcgattccacctacaaccttcgtgccgtggagctatccggcccaaggtag

SEQ ID NO: 103 - protein, 641 aa, Vitis Vinifera
  unsure (343)..(376): Xaa stands for any amino acid
MDPGIFGSVFVSLIIVFSAFLRVSGNSEGDALNALKSNLADPNNVLQSWDATLVNPCTWFHVTCNSDNSVTRVDLGNANLSGQLVSQLGQLTNLQYLELYSNNISGKIPEELGNLTNLVSLDLYMNKLSGPIPTTLAKLAKLRFLRLNNNTLTGTIPRSLTTVMTLQVLDLSNNQLTGDIPVDGSFSLFTPISFNNNRLNPLPVSPPPPISPTLTASSGNSATGAIAGGVAAGAALLFAAPAIVLAWWRRRKPQEHFFDVPAEEDPEVHLGQLKRFSLRELQVATDNFSNKHILGRGGFGKVYKGRLTDGSLVAVKRLKEERTQGGELQFQTEVEMISMAVHXXXXXXXXXXXXXXXXXXXXXXXXXXXXXXXXXXLRLHGFCMTPTERLLVYPFMVNGSVASCLRERADGQSPLNWPIRKQIALGSARGLAYLHDHCDPKIIHRDVKAANILLDEEFEAVVGDFGLAKLMDYKDTHVTTAVRGTIGHIAPEYLSTGKSSEKTDVFGYGVMLLELITGQRAFDLARLANDDDVMLLDWVKGLLKDKKLETLVDADLQGDYIEVEVEELIRVALLCTDGAAAQRPKMSEVVRMLEGDGLAERWEQWEKDDIIRQEYNHIPHPDSNWIDSTAGLRPDELSGPR

SEQ ID NO: 104 - DNA, 1926 bp, Vitis Vinifera
  unsure (1028)..(1127): n stands for any base
  misc_feature (1028)..(1127): n is a, c, g, or t
atggacccggggatcttcggttcggtttttgtttccttgattatagtattctctgcgtttctgagggtctctggtaattccgaaggtgatgctttgaatgcgttgaagtcaaatttagctgatcctaacaatgttttgcaaagttgggatgctacccttgtcaatccttgcacatggtttcatgttacatgcaacagtgacaatagtgttacaagagttgatcttggaaatgcaaatttatccggtcaactggtttcacagcttggtcagcttacaaatttgcaatatctggaactttatagtaataacataagtggcaaaataccagaggagcttgggaatttgacaaacttggtgagcttggatctttacatgaacaagttaagtggtccaattccgacgacgttggccaagcttgcaaaactacgtttcctgcggcttaacaacaacaccttgacaggaactattccaagatctttaactactgttatgacactgcaagtcctggatctttcaaacaatcagctaacaggagatataccagttgacggctcattttcattatttactcctatcagttttaacaataatagactaaacccacttccagtttctccaccaccaccaatttctcctacactaacagcttcttcaggaaacagtgccactggagccattgctggaggagttgctgctggtgctgcacttctgtttgctgcccctgcaatcgtacttgcctggtggcgacgaaggaaaccacaggagcacttttttgatgtacctgctgaagaggatccagaagttcatttgggacagcttaaaaggttttctctgcgtgaactacaagttgcaacggataattttagtaacaaacacattctgggtagaggtggatttggtaaagtttacaaaggacgtttaactgatggttctctagtggcagtaaaaagactgaaagaggagcgtactcagggtggggaactgcaatttcagacagaagtcgaaatgatcagcatggctgtgcaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacttcgtctacatggcttttgcatgacaccaacagaacggttgcttgtctatccctttatggttaatggaagtgttgcatcatgtttgagagagcgagctgatggacagtctccccttaattggccaataaggaaacaaattgctttaggatcagcaagggggcttgcttatttgcatgatcattgtgaccctaagattattcatcgtgatgtgaaggctgcaaacatattgttggatgaagagtttgaagcagtagtaggagactttgggctggctaaactcatggactacaaggatactcatgttaccactgctgtacgtggcacaattgggcatattgctccggagtacctctccactgggaagtcttcagagaagactgatgtttttggatatggagtaatgcttcttgagcttatcactggacagagggcttttgatcttgcgcggcttgccaatgatgatgatgttatgttacttgattgggtaaaaggactactgaaagataagaagttggagacactggttgatgccgatctgcagggtgattacattgaggtagaagtagaggagctaattcgggtggctctcctctgcacagatggtgctgctgcacaacgacccaaaatgtcagaggtggtcagaatgcttgaaggtgatggtttggccgagagatgggaacaatgggagaaggatgatatcatccgccaagagtacaaccatatcccccacccagactctaattggattgactccaccgcaggcctccgccctgatgaattgtctggtccgagatga

SEQ ID NO: 105 - protein, 662 aa, Vitis Vinifera
MNKLSSLRISKNIYSLIKNLQLLSGTVDFDAMYHNLDNHQHCNLAIGHGESSASLEGKYDKHILTGNGPESLAKTSRDPNNVLQSWDPTLVNPCTWFHVTCNSDNSVIRVDLGNAALSGQLVPQLGLLKNLQYLELYSNNISGPIPSDLGNLTSLVSLDLYLNSFTGPIPETLGKLSKLRFLRLNNNSLTGTIPMSLTNITALQVLDLSNNRLSGVVPDNGSFSLFTPISFANNLDLCGPVTGHPCPGSPPFSPPPPFDYSNLSFNISGGNSATGAIAGGVAAGAALLFAAPAIGFAWWRRRKPQEYFFDVPAEEDPEVHLGQLKRFSLRELQVATDSFSNKNILGRGGFGKVYKGRLADGSLVAVKRLKEERTPGGELQFQTEVEMISMAVHRNLLRLRGFCMTPTERLLVYPYMANGSVASCLRERPASEPPLDWPTRKRIALGSARGLSYLHDHCDPKIIHRDVKAANILLDEEFEAVVGDFGLAKLMDYKDTHVTTAVRGTIGHIAPEYLSTGKSSEKTDVFGYGIMLLELITGQRAFDLARLANDDDVMLLDWVKGLLKEKKLEMLVDPDLKNNYVEAEVEQLIQVALLCTQGSPMDRPKMSEVVRMLEGDGLAERWDEWQKVEVLRQEVELAPHSNSDWIVDSTDNLHAVELSGPR

SEQ ID NO: 106 - DNA, 1989 bp, Vitis Vinifera
atgaataaattgtcttctttaagaatttccaaaaatatttattccttaattaaaaatcttcagctcttaagtggaactgtggattttgatgccatgtatcataatcttgacaaccaccaacactgtaacctcgctattggccacggagagagtagcgcatctctggagggtaaatacgacaaacatatattgaccggaaatggacctgaaagtcttgccaagacatccagagacccaaacaatgtcctacagagttgggatccgacccttgtcaacccctgcacatggtttcatgttacctgcaatagtgataatagtgttataagagttgatcttggaaatgctgctttgtcgggtcaactggtaccacagcttggcctccttaagaatttgcagtacttggagctctacagtaataacataagtggaccaattcctagtgaccttgggaatctaactagcttggtgagcttggatctttatttgaacagttttactggtcccatcccggagacattgggcaagctatcaaagttgcgcttcctccggcttaacaacaacagcctgacgggtactattcctatgtcattgactaatatcacggctctgcaagtattggatctatcaaacaaccgcctctcaggagtggttccagacaatggctctttttcattatttacccccatcagttttgctaataacctggatttatgtggcccggttactgggcacccatgccctggatctcccccattttctccacctcccccatttgattattctaatctctctttcaacatttcaggagggaacagtgccacaggagcaattgctgggggtgtggctgctggtgctgctttactatttgctgctcctgcaattggttttgcatggtggcgccgaaggaaaccacaagaatatttctttgatgtacctgctgaagaggacccagaggttcatctggggcagcttaaaaggttttcactgcgagaattacaagttgcaacagatagttttagcaacaagaacattctgggtagaggtggatttggtaaggtgtacaaaggacgcttagcggatggttctcttgtggctgtgaaaagattgaaagaagagcgtacaccaggtggtgagctgcagtttcaaacagaggtagagatgataagcatggctgtgcatcggaatctcctccgtcttcgtggtttttgcatgacacctactgaacggctgcttgtttatccgtatatggctaatggaagcgttgcatcatgtttaagagaacgcccggcatctgaaccaccacttgattggccaacaaggaagcgaattgcgttgggatctgcaagagggctttcttatttgcatgatcattgtgacccaaagattattcaccgtgatgtgaaagctgcaaatattttgttggatgaggaatttgaggctgttgttggagactttgggttagctaagcttatggattacaaggatacccatgttaccactgctgtccgtggcacaataggacatatagctccagagtacctctctactggaaagtcttcagaaaaaactgatgttttcgggtatggaattatgcttctggagctaatcactgggcagagagcttttgatcttgctcggcttgccaatgatgatgatgtcatgttgcttgattgggtaaaaggacttctgaaagagaagaagttagaaatgctggttgatcctgatcttaagaacaattatgtagaagcagaagtagagcagctaatccaggttgccctgctgtgcacgcaaggctctccaatggaccggcccaagatgtcagaagtggtgagaatgctggaaggtgatggattggcagagaggtgggacgagtggcagaaagtggaagttctccgccaggaggtggaactcgctcctcactccaactctgattggattgtggactcaacagacaatctacatgcggttgaattatcgggtccaaggtga

SEQ ID NO: 107 - protein, 624 aa, Vitis Vinifera
MEREIGASFLVWLILFVRPLTMIYANMEGDALHSLRTNLEDPNNVLQSWDPTLVNPCTWFHVTCNNENSVIRVDLGNAALSGQLVPQLGQLKNLQYLELYSNNISGQIPSDLGNLTSLVSLDLYLNRFTGAIPDTLGKLTKLRFLRLNNNSLSGSIPMFLTNISALQVLDLSNNRLAGPVPDNGSFSLFTPISFANNLNLCGPVIGKPCPGSPPFSPPPPFVPPSTVSSPGGNSATGAIAGGVAAGAALLFAAPAIGFAWWRRRKPQEHFFDVPAEEDPEVHLGQLKRFSLRELQVATDSFSNKNILGRGGFGKVYKGRLADGSLVAVKRLKEERTPGGELQFQTEVEMISMAVHRNLLRLRGFCMTPTERLLVYPYMANGSVASCLRERPPSEPPLDWTTRKRIALGSARGLSYLHDHCDPKIIHRDVKAANILLDEEFEAVVGDFGLAKLMDYKDTHVTTAVRGTIGHIAPEYLSTGKSSEKTDVFGYGIMLLELITGQRAFDLARLANDDDVMLLDWVKGLLKEKKLEMLVDPDLQTNYVEAEVEQLIQVALLCTQGSPMERPKMSEVVRMLEGDGLAERWEEWQKVEVVRQEVELAPPRCSEWIVDSTDNLHAVELSGPR

SEQ ID NO: 108 - DNA, 1875 bp, Vitis Vinifera
atggagcgggagatcggtgcttcgtttctggtttggttgatcttgtttgttcgtccattgactatgatatatgctaatatggaaggtgatgctttgcatagcctaagaaccaatttagaagatcctaacaatgtgctgcagagttgggatcctacgctagtgaacccatgcacatggtttcatgtcacatgtaacaatgaaaatagtgttataagagttgatcttggaaatgcagcattatctggtcaattggttccacaacttggacagcttaagaatttacagtacctggaactttatagtaacaacataagtggacaaattcctagtgatcttgggaatctgacaagcttggtgagcttggatctctacttgaacaggttcactggtgccattccagacacattgggcaagctgacaaaactgcgcttcctccggcttaacaacaacagcctatcaggttccattcccatgttcttgactaatatctcagcactgcaagtcttggatctttcaaacaatcgtctagcaggacctgttccagacaatggttcattttcactatttactcccataagttttgcaaataacttgaatctatgtggtccggttattgggaagccctgccctgggtctcctccattttctccaccacctccatttgtgccaccatctacagtttcttctcctggaggaaatagtgccacaggagcaattgctggaggagtggctgctggtgctgctttactgtttgctgcaccagcaattggttttgcatggtggcggcggaggaagccacaagaacatttctttgatgtacctgctgaagaggatccggaagtccacctggggcagctcaaaaggttttccctgcgagaattacaagttgcaacagatagctttagcaacaaaaatattctggggagaggtggatttggaaaggtgtacaagggtcgccttgcagatggttcattagtggcagtgaagagacttaaagaagaacgtactccgggtggagagttgcagtttcagacggaagtagaaatgattagcatggctgtgcaccggaatctgcttcggttacgtggtttttgcatgacaccaactgagcggttacttgtttatccatacatggctaatggaagtgttgcttcatgcctaagagaacgtccaccatcagaaccaccacttgattggaccactaggaaaagaatagctttgggatctgcaagggggctttcctatttgcatgatcactgcgatccaaagataattcaccgtgacgtgaaagcagccaatattttgttggatgaggagtttgaagctgttgttggtgattttggattggctaaacttatggactataaggacacacatgttactactgctgtacgtggcaccattgggcatatagcgcctgagtatctctctacggggaaatcatcagagaagacagatgtttttggttatggaattatgcttttagagctaattactggacagagggcatttgatcttgctcgacttgcaaatgatgatgatgtcatgctgcttgattgggttaaagggcttctaaaggagaagaagttggaaatgttggttgatcccgatctgcagacaaattatgttgaagctgaagtagagcagctgatccaagtagcgctgctgtgcacacaagggtctccaatggaacggcctaagatgtccgaggttgtccgaatgcttgaaggtgatggtttggcagaaagatgggaggaatggcagaaggtggaggtcgtccgtcaggaggtggaactggcccctccccggtgttctgaatggatcgtagactccaccgacaacctacatgctgtagaattgtctggtccaagatga

SEQ ID NO: 109 - protein, 610 aa, Physcomitrella patens subsp. patens
MSSLLITAWDVGCAGDALNAFRQNLIDNGNVLQSWVPDLVNPCTWFYITCNDELNVIRVDLGNAGLSGTLVPQLGVLTKLQYLVLYSNNITGQIPKELGNISALVSLDLYQNNFTGPIPDSLGQLSNLRFLRLNNNSLTGSIPASLTAIQGLQVLDLSYNKLSGPVPTYGSFSLFTPISFLGNDGLCGSVVGKPCPGEPPFPPPPPFTPPPPQTKGQQTSTGAIAGGVAAGAALLFSIPAIAYAWWRRRRPLDAFFDVAAEEDPEMQLGQLRRHSLRELQVATDDFSDRNILGRGGFGMVYKGRLADGTLVAIKRLKEQRSPRGELQFQNEVEMISMAVHRNLLRLRGYCTSSTERLLVYPYMGNGSVASRLRERVDGERPLSWQTRKKIALGAARGLSYLHDHCDPKIIHRDVKAANILLDEEFEAVMGDFGLAKLMDYKDAHVTTAVVGTIGHIAPEYLSTGKSSEKTDVFGYGIFLLELVTGRRAFDLSGMANAGGAMLLDWVTNLLGEHKIYILVDPDLEKNYDEEEVEELIQVALLCTQGSPVDRPKMGDVVHILEGDGLAERWEEWQKVEVIRKQDYDMPTRQTSQWILDSTENLHAVELSGPR

SEQ ID NO: 110 - DNA, 1833 bp, Physcomitrella patens subsp. patens
atgtcaagcctgcttatcacggcgtgggatgttggctgtgcaggagatgcactcaatgcttttcggcaaaatttgattgataatggcaacgtgctgcagagttgggtgccagatcttgtaaacccctgcacttggttttatattacctgcaatgacgaattgaacgtcattcgagtggatttaggaaatgctggtttatcaggaacattagtgccgcaacttggggtccttacgaagctacaatacctggtgttgtatagtaacaacattactggtcaaattcctaaagagctgggaaacatcagtgctcttgttagtctggatctttatcaaaacaactttactggcccaataccagatagtcttggacagctgagcaatctccgatttcttcggttgaacaacaatagcttgaccggatccattcctgcttctcttaccgccattcaaggattgcaagtcttggatctctcgtacaataagttatctggacccgttcctacgtatggctccttttcattattcacacccatcagttttttgggaaatgacggtctgtgcggatcagtggtcggcaaaccgtgccctggagaacccccattcccacctcctcctccgttcacgcctccacctccacaaacgaaaggtcaacaaacaagtacaggagctattgcaggtggtgttgccgcaggtgccgctttgttattctcgattcctgccattgcttatgcgtggtggcgtcgccggaggccccttgatgccttctttgatgttgctgctgaagaagatccagagatgcaattaggacagcttagacgtcatagtctgagagagcttcaggtggcaacagacgacttcagcgacagaaacattttaggtcgtggtgggtttggaatggtttataaaggccgattagcagacgggacgctcgtggctatcaaacgcctcaaagagcaacgctctcctcgaggagagctgcagtttcaaaatgaggttgagatgattagtatggcagtacaccgaaatttattacggcttcgtggatactgcacttcctctaccgaacggttgcttgtgtacccgtacatggggaatgggagtgttgcctctcgattgcgagaacgtgtggatggagagcgccctctgagttggcagactaggaagaaaattgcactaggagctgcccgagggctatcgtacttgcacgatcattgtgacccaaaaattatccatcgtgatgttaaggctgccaacattctcttggacgaagaatttgaagcagtaatgggagattttggtcttgctaaactgatggattataaagacgcacatgtcacgactgctgtggttgggactatcggccacatagcaccagaatatctttccactggaaaatcgtcggagaagacggacgtttttgggtatggaatctttttactggagcttgtcactgggcgacgtgcttttgacttgtccggcatggctaatgctggtggtgcgatgctcttggattgggtgacaaatttattgggggaacataagatctatattctggtagaccctgatcttgagaaaaattacgatgaggaagaagttgaagagctaattcaggtagcactactctgcactcaaggctctccagtagatcggccaaaaatgggtgatgttgtgcacattctggaaggagatggtctagcagagcgctgggaggaatggcagaaggtggaagttattcggaagcaagactacgatatgccaacccgccaaacatcacaatggattttagattccacagagaatcttcatgctgttgaactttctggtccgagatga

SEQ ID NO: 111 - protein, 611 aa, Physcomitrella patens subsp. patens
MRCNRCCAGDALNALRQNLIDSSNVLQSWDPTLVNPCTWFHVTCNNENSVIRVDLGNAGLSGSLVPQLGVLTKLQYLELYSNNISGTVPKELGNITALVSLDLYQNNFTGTIPDSLGQLSNLRFLRLNNNSLTGPIPVSLTTITGLQVLDLSYNKLSGDVPTNGSFSLFTPISFLGNSDLCGAVVGKQCPGQPPFPPPPPFTPPPPQTPSGPYANNKQTISTGAIAGGVAAGAALLFAAPAIGFAWWRRRRPIEAFFDVPAEEDPEVHLGQLKRFSLRELQVASDNFNNRNILGRGGFGKVYKGRLADGTLVAIKRLKEERSPGGELQFQTEVEMISMAVHRNLLRLRGFCMTPTERLLVYPYMPNGSVASRLRERVDEEPALSWRTRKQIALGAARGLSYLHDHCDPKIIHRDVKAANILLDEEFEAVVGDFGLAKLMDYKDTHVTTAVRGTIGHIAPEYLSTGKSSEKTDVFGFGIMLLELITGQRAFDLARLANDDDVMLLDWVKGLLRERKVDLLVDPDLKQNYDQKEVEELIQVALLCTQGSPLDRPKMGDVVRMLEGDGLAERWEEWQKVEVVRNQDLDLPPHRTSEWIVDSTDNLHAVELSGPR

SEQ ID NO: 112 - DNA, 1836 bp, Physcomitrella patens subsp. patens
atgaggtgtaatcgttgctgtgcaggcgatgcactcaatgctttgcgccaaaatttgattgatagtagcaatgtgctgcagagttgggatcccacgcttgtgaatccctgcacttggttccatgttacctgcaataacgaaaatagcgtcattcgagtggatttagggaatgctggtttgtcagggtcgttggtgccacaacttggggtccttacaaagctccagtacttggagttatatagtaacaatatttcaggaacagtacctaaagagctgggaaacatcactgcccttgtcagcctggatctttatcaaaacaatttcaccggcactataccagatagtcttgggcagctgagcaatctacggtttctccgactgaacaacaacagcttgaccggccccattccagtttctctaacgactattactggattgcaagtgctggacctctcgtacaacaaattatctggagacgtgcctaccaatggttccttttcacttttcacacctatcagttttttgggaaacagcgatttgtgtggtgcagtggttggtaaacagtgtcctgggcagcccccattccctcctcctcctccattcacacctccgcctccacaaactccaagtgggccttatgcaaacaacaaacaaacaataagtacaggagctattgcaggtggagttgctgcaggggctgctttgctgtttgccgcgccagctataggttttgcctggtggcgtcgtcggaggcccattgaggcgttctttgatgtccctgctgaggaagatccagaagtgcacttaggacagctgaagcgcttttctttgagggagctccaggtggcatcagacaacttcaacaacaggaacatcctaggtcgtggtggatttggaaaggtttacaaaggtcggttagcagatgggacgctggtggctatcaagcgcctcaaggaagagcgcagtcctggaggagagctgcagtttcaaacagaggttgagatgattagtatggcggtgcaccggaatttattacgacttcgtggattctgtatgactcctacagagcgtttgcttgtgtatccatatatgccaaacggtagtgttgcctctcgactgcgagaacgtgtggatgaagagcctgccttaagttggcgaaccaggaagcaaatcgcattgggagctgcccgtgggctgtcatacttgcacgatcactgtgacccaaaaatcattcatcgtgatgttaaggctgccaacattctcttagatgaagaatttgaagcagtggttggagatttcggacttgctaaacttatggactataaggacacccatgtcacaacagcagtgcgagggactattggccacattgcaccagagtatctttccactgggaaatcttcagagaaaacagatgtttttgggtttggaatcatgcttttggagctcatcaccggtcagcgtgcctttgacttggcacgtttggccaatgatgacgatgtcatgctcttggattgggtgaaggggttgttgagggaacgcaaggttgatcttcttgtagatcccgatcttaagcaaaattatgatcaaaaggaagttgaagagcttattcaggttgcactgctttgcactcaaggctctcctttggaccggccaaaaatgggtgatgtcgtgcgtatgctcgaaggtgacggactagccgagcgctgggaggagtggcagaaggtggaagtcgttcgaaatcaagatttggatttgcccccccaccggacctcagagtggattgtagattcaacagataatctgcatgctgttgagctttctggcccacggtga

SEQ ID NO: 113 - protein, 626 aa, selaginella moellendorffii
MANIGILVLALLLRAVIRVYGNAEGDALHDLKTSLTDPSSVLQSWDSTLVNPCTWFHVTCDNDNFVTRVDLGNAALSGTLVPSLGRLSHLQYLELYSNNITGEIPPELGNLSNLVSLDLYQNNFTSSIPDTIGRLTKLRFLRLNNNSLSGSIPMSLTNINGLQVLDLSNNDLSGPVPTNGSFSLFTPISFNNNRDLCGQAVNKRCPNGPPLTPAPQYLAPPSGANNGRTQSSSSSNTGAIAGGVAAGAALLFAAPAIGFAWWRRRRPPEAYFDVPAEEDPEVHLGQLKRFSLRELQVATDGFSNKNILGRGGFGKVYKGRLSDGSLVAVKRLKEERSPGGELQFQTEVEMISMAVHRNLLRLRGFCMTPTERLLVYPYMANGSVASRLRERNPGEPSLDWPTRKRIALGSARGLSYLHDHCDPKIIHRDVKAANILLDEEYEAVVGDFGLAKLMDYKDTHVTTAVRGTIGHIAPEYLSTGKSSEKTDVFGYGIMLLELITGQRAFDLARLANDDDVMLLDWVKGLLREKKVVQLVDSDLHNTYDLGEVEELIQVALLCTQVSPNDRPKMADVVRMLEGDGLAERWEEWQKVEVVRNQEMDFVPQRASDWIIDSTDNLHAVELSGPR

SEQ ID NO: 114 - DNA, 1881 bp, selaginella moellendorffii
atggcaaatatcgggatcttggtgctggcgctgctgctccgcgccgtgattcgcgtctatggcaacgccgaaggtgatgctctccacgatttgaagacctcgctcacggacccgagcagtgttctccagagctgggactcgacgctggtgaatccctgtacttggttccatgttacttgcgacaatgataatttcgtcactagagtagatctcggtaatgcagctctgtcgggaacgctagttccatcacttggtcgtctaagccacttacagtacttggaactgtacagtaataacatcactggagaaattccacctgagctaggcaacttatccaacctggtgagcctagatttgtaccagaacaattttacatcttcgataccagatacaattgggcgtctgactaagcttagattccttcgtctcaacaacaattctctgtctgggagtattcccatgtcgcttacaaatataaacggcttgcaagtacttgatctttctaacaatgatctatctgggccagttcctacgaatggatccttctccctgttcactcctatcagtttcaacaataatagagatctttgtggacaagcagtcaataaacgatgtcccaacggcccacctttgactcctgcacctcaatatttagcaccgccatctggagcaaacaatgggagaacacagtcatcaagctcgtcaaacactggagctattgctggtggagttgctgctggcgctgctcttctttttgctgctccggccattggatttgcttggtggagaagacggaggccaccggaagcttactttgacgttcctgctgaagaagaccccgaagttcatttaggacaactaaaaagattctctctgcgagaactgcaagtggctaccgatggttttagtaacaaaaacatccttgggagaggtggttttggcaaagtgtacaaggggagattgtcggatggctcactagtagccgttaaaaggcttaaagaagaacgcagtccgggtggagagctgcaatttcaaacagaagtggaaatgatcagtatggctgttcatcgaaaccttcttcgcctacgaggtttctgcatgactcccaccgaacgcttactcgtatatccatatatggcgaatggaagtgtagcatctcggctaagagaacgaaatccaggggagccatcacttgattggcctacccgaaagcgcattgctttgggatcagcaagaggcttgtcttacctgcacgaccattgtgatcccaaaatcatccatcgcgatgtaaaagcggcaaacatcctgctagacgaggaatacgaagcagtggttggtgattttggactggcaaagctcatggattacaaagacacgcacgtaacaactgccgttcgtggtaccattggtcacatcgcgccagagtacttgtccaccggcaagtcgtccgagaaaacagacgtcttcgggtacggtatcatgcttctggagctcatcaccgggcaaagggccttcgatcttgcacgattggcgaatgacgacgatgtgatgcttctcgactgggtgaaaggacttctccgggagaaaaaagtcgtccagctcgtcgattccgatcttcacaacacgtacgacttgggtgaagtcgaggaactcatccaggtggctttgctctgcacgcaagtctctcccaacgacaggccaaagatggcagacgtcgtccggatgctcgaaggcgacggccttgccgagcgatgggaagaatggcaaaaggtggaggttgtccgcaaccaagagatggatttcgtcccccagagagcatcagactggatcatcgactccacggacaatcttcacgcggtggagctgtctgggccgagatag

SEQ ID NO: 115 - protein, 626 aa, selaginella moellendorffii
MANIGILVLALLLRAVIRVYGNAEGDALHDLKSSLMDPSSVLQSWDSTLVNPCTWFHVTCDNDNFVTRVDLGNAALSGTLVPSLGRLSHLQYLELYSNNITGEIPPELGNLSNLVSLDLYQNNFTSSIPDTIGRLTKLRFLRLNNNSLSGSIPMSLTNINGLQVLDLSNNDLSGPVPTNGSFSLFTPISFNNNRDLCGQAVNKRCPNGPPLTPAPQYLAPPSGANNGRTQSSSSSNTGAIAGGVAAGAALLFAAPAIGFAWWRRRRPPEAYFDVPAEEDPEVHLGQLKRFSLRELQVATDGFSNKNILGRGGFGKVYKGRLSDGSLVAVKRLKEERSPGGELQFQTEVEMISMAVHRNLLRLRGFCMTPTERLLVYPYMANGSVASRLRERNPGEPSLDWPTRKRIALGSARGLSYLHDHCDPKIIHRDVKAANILLDEEYEAVVGDFGLAKLMDYKDTHVTTAVRGTIGHIAPEYLSTGKSSEKTDVFGYGIMLLELITGQRAFDLARLANDDDVMLLDWVKGLLREKKVVQLVDSDLHNTYDLGEVEELIQVALLCTQVSPNDRPKMADVVRMLEGDGLAERWEEWQKVEVVRNQEMDFVPQRASDWIIDSTDNLHAVELSGPR

SEQ ID NO: 116 - DNA, 1881 bp, selaginella moellendorffii
atggcaaatatcgggatcttggtgctggcgctgctgcttcgcgccgtgattcgtgtctatggcaacgccgaaggtgatgctctccacgatttgaagagctcgctcatggacccgagcagtgttctccagagctgggactcgacgctggtgaatccctgtacttggttccatgttacttgcgacaatgataatttcgtcactagagtagatctcggtaatgcagctctgtcgggaacgctagttccatcacttggtcgtttaagccacttacagtacttggaactgtacagtaataacatcactggagaaattccacctgagctaggcaacttatccaacctggtgagcctagatttgtaccagaacaattttacatcttcgataccagatacaattgggcgtctgactaagcttagattccttcgtctcaacaacaattctctgtctgggagtattcccatgtcgcttacaaatataaacggcttgcaagtacttgatctttctaacaatgatctatctgggccagttcctacgaatggatccttctccctgttcactcctatcagtttcaacaataatagagatctttgtggacaagcagtcaataaacgatgtcccaacggcccacctttgactcctgcacctcaatatttagcaccgccatctggagcaaacaatgggagaacacagtcatcaagctcgtcaaacactggagctattgctggtggagttgctgctggcgctgctcttctttttgctgctccggctattggatttgcttggtggagaagacggaggccaccggaagcttactttgacgttcctgctgaagaagaccccgaagttcatttaggacaactaaaaagattctctctgcgagaactgcaagtggctaccgatggttttagtaacaaaaacatccttgggagaggtggttttggcaaagtgtacaaggggagattgtcggatggctcactagtagccgttaaaaggcttaaagaagaacgcagtccgggtggagagctgcaatttcaaacagaagtggaaatgatcagtatggctgttcatcgaaaccttcttcgcctacgaggtttctgcatgactcccaccgaacgcttacttgtatatccatatatggcgaatggaagtgtagcatctcggctaagagaacgaaatccaggggagccatcacttgattggcctacccgaaagcgcattgctttgggatcagcaagaggcttgtcttacctgcacgaccattgtgatcccaaaatcatccatcgcgatgtaaaagcggcaaacatcctgctagacgaggaatacgaagcagtggttggtgattttggactggcaaagctcatggattacaaagacacgcacgtaacaactgccgttcgtggtaccattggtcacatcgcgccagagtacttgtccaccggcaagtcgtccgagaaaacagacgtcttcgggtacggtatcatgcttctggagctcatcaccgggcaaagagccttcgatcttgcacgattggcgaatgacgacgatgtgatgcttctcgactgggtgaaaggacttctccgggagaaaaaagtcgtccagctcgtcgattccgatcttcacaacacgtacgacttgggtgaagtcgaggaactcatccaggtggctttgctctgcacgcaagtctctcccaacgacaggccaaagatggcagacgtcgtccggatgctcgaaggcgacggccttgccgagcgatgggaagaatggcaaaaggtggaggttgtccgcaaccaagagatggatttcgtcccccagagagcatcagactggatcatcgactccacggacaatcttcacgccgtggagctgtctgggccgagatag

SEQ ID NO: 117 - protein, 612 aa, selaginella moellendorffii
MELESHVIAIVRARALTCVALDDPSNVLQSWDPTLVNPCTWFHVTCNTQDNVIRVDLGNAFLSGRLVAALGNLENLQYLELYSNNITGPIPKELGNLTELVSLDLYQNSFTGDIPDSLGKLHNLRFLRLNNNTLDGKIPNSLTTIPGLQVLDLSNNNLSGPVPTNGSFSLFTPISFGGNPALCGAVVSRQCPGGPPLPPPTPYQPPSPFVGNQNGNNGGSSSTGAIAGGVAASAALLFATPAIAFAWWKRRRPHEAYFDVPAEEDPEVHLGQLKRFSLRELQVATDNFNNRNILGRGGFGKVYKGRLADGSLVAVKRLKEERSPGGELQFQTEVEMISMAVHRNLLRLRGFCMTPTERLLVYPYMPNGSVASRLRERLPGDTPLDWPTRKCIALGAARGLSYLHDHCDPKIIHRDVKAANILLDEEYEAVVGDFGLAKLMDYKDTHVTTAVRGTIGHIAPEYLSTGKSSEKTDVFGFGIMLLELITGQRAFDLARLANDDDVMLLDWVKGLLRERKVDLLVDPDLKNEYDPMEVEQLIQVALLCTQGSPMDRPKMAEVVRMLEGDGLAERWEEWQKVEVVRSQEVELVSHGNSEWIVDSTDNLHAVELSGPR

SEQ ID NO: 118 - DNA, 1839 bp, selaginella moellendorffii
atggaactcgaatcgcatgtcattgcaatcgtgagagcgcgggcactgacttgtgtggcgctggatgatcctagcaacgtcttgcaaagctgggatcccacactggtcaatccttgtacctggtttcacgtaacttgcaacacccaagacaacgttataagagtagacttgggaaatgcatttctctcagggcgtttggtagcagctcttggcaatctcgaaaatttacagtacttggagttgtacagcaacaacatcactgggccaatcccaaaggagctgggcaacttgactgagctcgtcagccttgatttgtaccaaaacagcttcactggcgatatacctgactcactcggtaaacttcataatctgaggttcctccgactaaataataatacgctcgacggcaagatccccaactcactcaccacaatcccggggcttcaagtgctggatctctcgaataacaatttgtcaggtccagttccaactaatggctccttttcgctgttcacgccgataagttttggaggtaatccggcattgtgtggtgctgttgtcagccgtcaatgtccaggagggcccccattgccacctccgactccctaccagccaccctcaccttttgttggcaatcaaaatggaaataatgggggatcctcgagcaccggtgccattgctggaggtgtggctgccagcgctgccttgctttttgcaactccagctatcgcttttgcgtggtggaaacgtcgcagaccgcacgaggcctacttcgatgtcccagctgaagaggatcccgaagttcaccttggtcagttgaagagattctcactccgagaacttcaggtcgcgacagataacttcaacaaccggaacatccttggtcggggtgggtttggaaaagtgtacaaaggcaggctggcagatggatctctggtagctgttaaaagattgaaggaagaacgaagtccaggtggtgagttgcagttccagactgaagtcgagatgataagtatggctgtacacagaaatcttctccgtttgcgtggtttttgcatgacaccaacggaaagacttcttgtttatccatacatgcccaacggaagcgttgcttcccggttacgagagagacttccgggagatacaccactggactggccaacgagaaaatgcatagctctaggtgcggcgcgtggtttgtcatatcttcacgaccactgtgatcccaaaattatccatcgcgacgtcaaagcagcaaacatcttgctggatgaggaatacgaagctgtggtgggggactttggcctggccaagctcatggactacaaagacacgcacgtcacaacagcagtccgaggcacgatcggccatatcgctcccgagtacttatccacggggaagtcgtcagagaaaacggacgtgtttggtttcgggatcatgttactggaactcatcactggacaacgagcgtttgatctcgctcgcctcgccaacgatgacgacgtgatgcttctcgactgggtgaaaggactgctaagagagcgaaaggtggaccttttagtggatcccgacttgaaaaacgagtacgatcccatggaagtcgagcagctcattcaggtggcacttctatgcacacaaggctctccaatggacaggcccaagatggccgaggtcgtaagaatgctcgaaggcgacggcctggcggagagatgggaagaatggcaaaaggtggaagttgtccgaagccaggaagtcgagctcgtgtcgcatggaaactccgagtggattgtcgactccacggataatctacacgcggtggaactctcgggtccaagatag

SEQ ID NO: 119 - DNA, 1013 bp, Artificial Sequence
  Synthetic polynucleotide, GmMYB74
gcggccgcatgggaagaccaccttgttgtgacaaagaaggggtcaagaaagggccttggactcctgaagaagacatcatattggtgtcttatattcaggaacatggtcctggaaattggagggcagttcctgccaaaacagggttgtcaagatgcagcaagagttgcagacttagatggacgaattacctgaggccaggaatcaagcgtggtaacttcacagaacaagaggagaagatgataatccatcttcaagatcttttaggaaacagatgggctgcaatagcttcataccttccacaaagaacagacaatgacataaagaactattggaatacccatttgagaaagaagctgaagaagatgcaagcaggcggtgaaggtggtagctttggagaagggttttcagcctcaaggcaaatccctagaggccagtgggaaagaaggctccaaactgatatccaaatggcaaagagagccctcagtgaagctctttcaccagagaaaaagccatcttgtttatctgcctcaaactcaaacccttcagatagtagcagctccttctcttccacaaaaccaacaacaacacaatctgtgtgctatgcatcaagtgctgacaacatagctagaatgctcaagggttggatgaagaacccaccaaagtcctcaagaaccaactcgtctatgactcagaactcattcaacaacttagcaggtgctgatactgcttgtagtagtggagcaaagggaccactaagcagtgccgaattgtctgagaataattttgaatccttgtttgattttgatcagtctttggagtcttcaaactctgatcaattctctcagtccttgtctcctgaggccactgttttgcaagatgaaagcaagcctgatattaatattgctgcagaaattatgcccttctctttgcttgagaaatggctccttgatgaggcaggttgccaagagaaattagttggttgttgtggtgatgccaagtttttctaagttaac

SEQ ID NO: 120 - DNA, 1008 bp, Artificial Sequence
  Synthetic polynucleotide, GmMYB74
ggccgcatgggaagaccaccttgttgtgacaaagaaggggtcaagaaagggccttggactcctgaagaagacatcatattggtgtcttatattcaggaacatggtcctggaaattggagggcagttcctgccaaaacagggttgtcaagatgcagcaagagttgcagacttagatggacgaattacctgaggccaggaatcaagcgtggtaacttcacagaacaagaggagaagatgataatccatcttcaagatcttttaggaaacagatgggctgcaatagcttcataccttccacaaagaacagacaatgacataaagaactattggaatacccatttgagaaagaagctgaagaagatgcaagcaggcggtgaaggtggtagctttggagaagggttttcagcctcaaggcaaatccctagaggccagtgggaaagaaggctccaaactgatatccaaatggcaaagagagccctcagtgaagctctttcaccagagaaaaagccatcttgtttatctgcctcaaactcaaacccttcagatagtagcagctccttctcttccacaaaaccaacaacaacacaatctgtgtgctatgcatcaagtgctgacaacatagctagaatgctcaagggttggatgaagaacccaccaaagtcctcaagaaccaactcgtctatgactcagaactcattcaacaacttagcaggtgctgatactgcttgtagtagtggagcaaagggaccactaagcagtgccgaattgtctgagaataattttgaatccttgtttgattttgatcagtctttggagtcttcaaactctgatcaattctctcagtccttgtctcctgaggccactgttttgcaagatgaaagcaagcctgatattaatattgctgcagaaattatgcccttctctttgcttgagaaatggctccttgatgaggcaggttgccaagagaaattagttggttgttgtggtgatgccaagtttttctaagtt

SEQ ID NO: 121 - DNA, 27 bp, Artificial Sequence
  Synthetic oligonucleotide primer
attagtcgacatggtgaggcctccttg

SEQ ID NO: 122 - DNA, 30 bp, Artificial Sequence
  Synthetic oligonucleotide primer
ttatgcggccgctcagaagaaattagtgtt

SEQ ID NO: 123 - protein, 104 aa, Arabidopsis thaliana
VKKGPWTPEEDIILVTYIQEHGPGNWRAVPTNTGLLRCSKSCRLRWTNYLRPGIKRGNFTEHEEKMIVHLQALLGNRWAAIASYLPQRTDNDIKNYWNTHLKKK

SEQ ID NO: 124 - protein, 104 aa, Glycine max
VKKGPWTPEEDIILVSYIQEHGPGNWRAVPAKTGLSRCSKSCRLRWTNYLRPGIKRGNFTEQEEKMIIHLQDLLGNRWAAIASYLPQRTDNDIKNYWNTHLRKK

(19) United States
(12) Patent Application Publication
(10) Pub. No.: US 2018/0135067 A1
(43) Pub. Date: May 17, 2018

(54) PLANT BODY IDEAL FOR HIGH-DENSITY PLANTING AND USE THEREOF

(71) Applicant: TOYOTA JIDOSHA KABUSHIKI KAISHA, Toyota-shi, Aichi (JP)

(72) Inventors: Kenichi OGAWA, Kaga-gun, Okayama (JP); Satoshi KONDO, Miyoshi-shi, Aichi (JP); Chikara OHTO, Toyota-shi, Aichi (JP); Soichiro NODA, Kaga-gun, Okayama (JP); Aya YASUKOCHI, Kaga-gun, Okayama (JP)

(73) Assignee: TOYOTA JIDOSHA KABUSHIKI KAISHA, Toyota-shi, Aichi (JP)

(21) Appl. No.: 15/311,369

(22) PCT Filed: Jul. 28, 2015
(86) PCT No.: PCT/JP2015/071380

§ 371 Date: Nov. 15, 2016

(30) Foreign Application Priority Data
Jul. 31, 2014 (JP) 2014-157155
Jul. 27, 2015 (JP) 2015-148125

(51) Int. Cl. C12N 15/82; C07K 14/415; C12Q 1/6895

(52) U.S. Cl. CPC C12N 15/8261; C07K 14/415; C12Q 2600/13; C12Q 1/6895; C12N 15/8279

(57) ABSTRACT

In order to improve biomass productivity per unit area by extending the limit of high-density planting, the present invention produces plant biomass by cultivating, under a high-density planting condition, a plant body transformed with an exogenous gene which contains an MYB30-related gene.

### TECHNICAL FIELD

The present invention relates to a plant body suitable for high-density planting and use of the plant body.

### BACKGROUND ART

It has been known that in general, when the number of individuals planted per unit area (hereinafter, referred to as “planting density”) increases, the weight of a plant individual decreases. Meanwhile, it is also known that when the planting density is increased, both yield and total biomass quantity per unit area increase. For example, in the case of Glycine max, cultivation at a high planting density is effective for increasing the yield of Glycine max. Accordingly, a method of cultivation at a high planting density is prevailing in the field of agriculture.

Cultivation at a high planting density for the purpose of increasing yield leads to an increase in biomass quantity per unit area. However, such cultivation accelerates competition between individuals at an earlier stage of growth. This results in rank growth and consequently causes the yield to level off. In other words, as the planting density increases, the biomass quantity per plant individual decreases. Accordingly, the biomass quantity per unit area levels off in due course. Non-Patent Literature 1 discloses that an increase in planting density leads to a decrease in weight of an individual, and a relationship between the weight “W” of an individual and the number “N” of plants per area (planting density) is expressed by the following:

log W=−3/2 log N [Chem. 1]

(i.e., “−3/2 power law”). In this way, Non-Patent Literature 1 discloses that a slope of a logarithmic graph showing a relationship between planting density and weight of a plant individual is constant.

Further, the following techniques are well known: (i) a technique for increasing a ratio of a biomass quantity of harvests to a total biomass quantity of plants (Patent Literature 1); and a technique for sufficiently increasing biomass quantity of plants per unit area (Patent Literature 2).

### CITATION LIST

### Patent Literatures

[Patent Literature 1]
Pamphlet of International Publication No. WO2008/072602 (published on Jun. 19, 2008)
[Patent Literature 2]
Pamphlet of International Publication No. WO 2008/087932 (published on Jul. 24, 2008)

### [Non-Patent Literature]

[Non-patent Literature 1] Lack and Evans (2001) Plant Biology 175-179, BIOS Scientific Publishers Limited

### SUMMARY OF INVENTION

### Technical Problem

As described above, each plant has an optimal planting density for biomass productivity per unit area. Then, even if plants are planted at a planting density higher than the optimal planting density, the biomass productivity per unit area of the plants does not improve. Accordingly, in order to improve the biomass productivity per unit area, it is necessary to extend the upper limit of yield in cultivation at a high planting density. Further, it is also known that an increase in yield obtained by cultivation at a high planting density varies depending on varieties of plants. Accordingly, there is a demand for breeding of a plant variety suitable for cultivation at a high planting density, as means for increasing the yield.

### Solution to Problem

The present invention provides a method and a tool each for producing plant biomass by means of cultivation at a high planting density, and use of the method and the tool. The present invention provides a technique for increasing yield more than ever before in cultivation at a high planting density, by changing the slope of the graph disclosed in Non-Patent Literature 1.

A method for producing plant biomass in accordance with the present invention includes the step of cultivating a plant body in which an MYB30 signaling pathway is activated, the plant body being cultivated under a high-density planting condition.

The method in accordance with the present invention is arranged preferably such that the plant body is a transformed plant obtained by transformation with an exogenous gene which contains an MYB30-related gene. In one embodiment, the MYB30-related gene may be operably connected to a promoter which regulates expression timing. In this case, the promoter is preferably arranged to initiate expression of the MYB30-related gene immediately prior to a flower bud formation stage of a non-transformed plant.

Preferably, the method in accordance with the present invention further includes the step of collecting biomass after cultivation of the plant body. For example, the method may further include the step of collecting biomass after fruiting of the plant body. For another example, the method may further include the step of collecting biomass prior to the flower bud formation stage.

Preferably, the method in accordance with the present invention is arranged such that the MYB30-related gene is a gene encoding a protein functionally equivalent to a protein selected from the group consisting of AtMYB30, BAK1 and PLA₂α.

A kit in accordance with the present invention includes an exogenous gene which contains an MYB30-related gene, for improving productivity per unit area of a plant under a high-density planting condition. The kit in accordance with the present invention may further include a reagent for determining the presence or absence of disease resistance which results from activation of an MYB30 signaling pathway.

In the exogenous gene, the MYB30-related gene may be operably connected to a promoter which regulates protein expression timing, and the MYB30-related gene is preferably a gene encoding a protein functionally equivalent to a protein selected from the group consisting of AtMYB30, BAK1 and PLA₂α.

A method for preparing a transformed plant in accordance with the present invention includes the step of transforming a plant body with an exogenous gene which contains a gene selected by screening with use of the kit. The method for preparing a transformed plant in accordance with the present invention may further include the step of selecting an individual in which the disease resistance is improved, the disease resistance resulting from activation of the MYB30 signaling pathway.

A screening method in accordance with the present invention includes, for screening a plant body having an improved productivity per unit area under a high-density planting condition, the steps of: comparing, with a reference value, an expression level of an MYB30-related gene or an expression level of a protein encoded by the MYB30-related gene; and selecting an individual whose expression level of the MYB30-related gene or of the protein encoded by the MYB30-related gene is higher or lower than the reference value (whose expression level has a significant difference from the reference value). Meanwhile, a screening method in accordance with the present invention includes, for screening a plant body having an improved productivity per unit area under a high-density planting condition, the steps of: comparing, with a reference value, an activation level of a protein encoded by an MYB30-related gene; and selecting an individual whose activation level of the protein is higher or lower than the reference value (whose activation level of the protein has a significant difference from the reference value). The screening method in accordance with the present invention may further include the step of selecting an individual having an improved disease resistance which results from activation of an MYB30 signaling pathway.

### Advantageous Effects of Invention

Use of the present invention makes it possible to obtain a plant body suitable for high-density planting and thereby to increase yield of plant biomass.

### BRIEF DESCRIPTION OF DRAWINGS

FIG. 1 is a graph that shows respective expression levels of MYB30 genes of transformed plants (18-1, 15-1, and 3-1) four weeks after sowing relative to an expression level of an MYB30 gene of a wild type (Col-0) four weeks after sowing.

FIG. 2 is a log-log graph showing a relationship between fresh weight of aerial part of and planting density of each of the wild type (Col-0) and the MYB30 transformed plant (3-1).

FIG. 3 is a graph for comparing power exponents a indicative of respective slopes in a log-log graph that shows a relationship between fresh weight of aerial part of and planting density of each of a wild-type strain and transformed plants.

FIG. 4 is a graph showing a correlation between (a) expression levels of MYB30 genes determined by real-time PCR and (b) the slopes a in the log-log graph showing the relationship between the fresh weight of and the planting density of each plant.

FIG. 5 is a chart showing results of comparison between the wild type (Col-0) and each of the MYB30 transformed plants ((a) 18-1, (b) 15-1, and (c) 3-1), in regard to a relationship between yield of biomass (fresh weight of aerial part) per pot and planting density.

FIG. 6 is a log-log graph showing a relationship between dry weight of aerial part of and planting density of each of the wild type (Col-0) and a GmMYB74 transformed plant (#3-2 strain).

FIG. 7 is a graph showing results of comparison between wild-type Oryza sativa and transformed Oryza sativa, in regard to a relationship between yield of biomass (fresh weight of aerial part) per pot and planting density.

### DESCRIPTION OF EMBODIMENTS

[1: MYB30-Related Gene]

myb genes are a group of genes widely present in eukaryotes, and are often present in plants. The myb genes encode MYB proteins which are transcription factors each having an MYB domain. It is known that a large number of MYB proteins are present in plants, and such MYB proteins are considered to regulate expression of various genes and to be thereby involved in various regulations/controls in cells.

AtMYB30 (At3g28910), which is one of MYB proteins (MYB transcription factors) of Arabidopsis thaliana is a transcription factor classified into an R2R3 type, in accordance with a repetitive sequence pattern in a C-terminal region. For example, in Arabidopsis thaliana, 125 R2R3-type transcription factors are present and AtMYB30 is classified into subgroup 1.

AtMYB30 is identified as a transcription factor involved in hypersensitive response of a plant and cell death of the plant, and known to contribute to an interaction between the plant and a pathogen, specifically, resistance (hypersensitive response) to an infection by pathogenic bacteria (Xanthomonas campestris, Pseudomonas syringe, etc.). It is also known that synthesis of a very long chain fatty acid (VLCFA) following activation of AtMYB30 is involved in the hypersensitive response of the plant (see, for example, Daniel et al. (1999) The Plant Journal 20(1): 57-66; Raffaele et al. (2008) The Plant Cell 20: 752-767; Reina-Pinto et al. (2009) The Plant Cell 21: 1252-1272; and the like). Further, it is also known that release of hydrogen peroxide is associated with the hypersensitive response (see, for example, Breusegem et al. (2006) Plant Physiology 141: 381-390; and Reina-Pinto et al. (mentioned above)). Further, AtMYB30 is also known to function downstream of the transcription factor called BES1, and reported to be involved in a signaling pathway of brassinosteroid which is a plant hormone. Further, Li et al. (2009) The Plant Journal 58: 275-28 describes that bri-1, which is a brassinosteroid-sensitive mutant, exhibits dwarfness and that knockout of AtMYB30 in bri-1 enhances dwarfness of bri-1. Furthermore, Daniel et al. (mentioned above) suggests that MYB30 plays an important role at an early stage of plant development. In addition, it is known that the amount of endogenous MYB30 is regulated by MIEL1 which is a ubiquitin E3 ligase (Marino et al. (2013) Nature Communications 4: 1476). However, there has been no report on the knowledge that AtMYB30 is associated with planting density.

The “planting density” as used in the present specification means the number of individuals planted per unit area. Generally, in a case where plants are grown, seedlings or young plants are planted or thinned at appropriate intervals. This is because when a planting density for individuals increases, biomass productivity per individual decreases and the biomass productivity per unit area levels off. As such, each plant has an optimal planting density for its biomass productivity per unit area. Planting of the plant at a planting density higher than the optimal planting density causes a decrease in crop yields with respect to purchase costs of seeds or seedlings, and therefore such planting is not preferable.

Biomass ethanol obtained by ethanol fermentation of starch sugar from Saccharum officinarum, Zea mays, or the like is an extremely important lower class alcohol fuel associated with reduction of carbon dioxide emission. Further, use of wood-based biomass such as arbor-based biomass is drawing attention, and there has been advancement in development of techniques for producing ethanol from arbor-derived glucose and techniques for producing monosaccharides or oligosaccharides from lignocellulose composed of cellulose and lignin.

The “biomass” is intended to mean renewable and biologically derived organic resources which exclude fossil resources. When the biomass is burned, carbon dioxide is emitted. However, this carbon dioxide is considered to cause no increase in the amount of carbon dioxide in the atmosphere. This is because the carbon dioxide emitted by burning the biomass originates from carbon dioxide which has been absorbed from the atmosphere during photosynthesis in a growth process of plants. Accordingly, an improvement in productivity of biomass is very effective for a shift of resources from fossil resources.

The “high-density planting” as used in the present specification is intended to mean planting at a planting density higher than the optimal planting density for the biomass productivity per unit area. Such a planting density is a planting density that sufficiently increases the biomass quantity per unit area. The “planting density that sufficiently increases the biomass quantity per unit area” means an optimal planting density for each variety (that is, an optimal planting density at which the biomass productivity per unit area is the highest). Further, though the optimal planting density varies depending on species of plants, a person skilled in the art can easily know an optimal planting density for each plant which is to be used. Furthermore, in the present specification, planting at the optimal planting density for the biomass productivity per unit area is referred to as “optimal-density planting”, and planting at a density lower than the optimal planting density is referred to as “low-density planting”.

The “biomass quantity” as used in the present specification is intended to mean the dry weight or production amount of a plant individual. The increase in biomass quantity leads to various beneficial effects as follows: (i) the amount of CO₂ in the atmosphere is efficiently reduced because carbon dioxide can be fixed as carbohydrate; (ii) in the case of vegetables, eatable portions of the vegetables increase and accordingly, food production is increased; (iii) in the case of timber and the like, production of raw materials for paper etc. can be increased; and the like.

The term “MYB30-related gene” as used in the present specification is intended to mean a gene encoding an MYB30-related protein, while the term “MYB30-related protein” is intended to mean an AtMYB30-like protein (protein functionally equivalent to AtMYB30 or AtMYB30), a protein which can positively regulate the expression or function of the AtMYB30-like protein, or a protein which functions downstream of the AtMYB30-like protein in a signaling pathway of the AtMYB30-like protein (hereinafter, also referred to as “MYB30 signaling pathway”).

The term “protein” as used in the present specification is used interchangeably with “peptide” or “polypeptide”. Further, the term “gene” as used in the present specification is used interchangeably with “polynucleotide”, “nucleic acid”, or “nucleic acid molecule”, and intended to mean a nucleotide polymer.

As shown in Examples described later, it was confirmed by a result of screening in which activation tag lines of Arabidopsis thaliana was used, that a plant body having an activated AtMYB30 is advantageous to high-density planting. This suggested that a function similar to that of AtMYB30 in terms of high-density planting is exhibited by gene products (e.g., BAK1, BR11, BES1, MIEL1, etc.) which can positively regulate the expression or function of AtMYB30, or gene products (e.g., PLA₂α, KCS1, FDH, etc.) which function downstream of AtMYB30 in the MYB30 signaling pathway.

PLA₂α is known to interact with AtMYB30 in Arabidopsis thaliana in vivo. Further, AtMYB30 is known to be involved in transfer of PLA₂α from cytoplasmic vacuoles to the nucleus. Furthermore, it has been shown that PLA₂α exchanges very long chain fatty acids (VLCFAs) between phospholipids and an acyl-CoA pool, and is thereby involved in hypersensitive cell death (Raffaele et al. (mentioned above); and Reina-Pinto et al. (mentioned above)). BAK1 is known to bind to BRI1, which is one of leucine-rich repeat receptor kinases. Further, BRI1 is known to induce expression of BES1, which is a transcription factor, and this BES1 is known to be involved in the function of MYB30 (Li et al. (mentioned above)). The above reports support that in high-density planting, PLA₂α and BAK1 exhibit effects similar to that of AtMYB30. Indeed, in Examples described later, BAK1 and PLA₂α are found in the vicinity of an enhancer in the result of screening with use of activation tag lines of Arabidopsis thaliana.

As described above, use of a gene encoding PLA₂α or BAK1 is considered to make it possible to obtain a plant body advantageous to high-density planting.

In one embodiment, the “MYB30-related gene” is intended to mean a gene encoding a protein which regulates the MYB30 signaling pathway, and also to mean a gene which encodes proteins that activate the MYB30 signaling pathway, that is, (a) an AtMYB30-like protein and (b) a protein that positively regulates (upregulates) the MYB30 signaling pathway upstream or downstream of the AtMYB30-like protein. Examples of the protein capable of positively regulating the expression or function of AtMYB30 encompass BES1 and BAK1, while examples of the protein which functions downstream of AtMYB30 encompass PLA₂α. However, the proteins that activate the MYB30 signaling pathway are not limited to the above examples. In one embodiment, the MYB30-related gene can be a gene encoding an AtMYB30-like protein, a PLA₂α-like protein (PLA₂α or protein functionally equivalent to PLA₂α) or a BAK1-like protein (BAK1 or protein functionally equivalent to BAK1).

The proteins of AtMYB30, BAK1 and PLA₂α of Arabidopsis thaliana have amino-acid sequences represented by SEQ ID NOs: 11, 13 and 21, respectively, and the genes respectively encoding these proteins have base sequences represented by SEQ ID NOs: 12, 14 and 22, respectively. Genes functionally equivalent to the above genes can be obtained by referring to known literatures and databases. These functionally equivalent genes thus obtained are also suitably used in the present invention.

As disclosed in Dubos et al. (2010) TRENDS in Plant Science 15(10): 573-581, MYB transcription factors belonging to one subgroup are known to fulfill a similar function each other. As described above, AtMYB30 is classified into an MYB transcription factor, which belongs to subgroup 1. Accordingly, AtMYB31 (At1g74650), AtMYB60 (At1g08810), AtMYB94 (At3g47660), and AtMYB96 (At5g62470), which belong to subgroup 1 of Arabidopsis thaliana, can be suitably used, similarly to AtMYB30, as MYB30-related proteins for the present invention. Note that a transcription factor functionally equivalent to AtMYB30 is not limited to the above transcription factors, and encompasses transcription factors (hereinafter, referred to as homologous transcription factors) which are in plants other than Arabidopsis thaliana and have a function similar to that of AtMYB30. Examples of such a transcription factor (AtMYB30-like protein) functionally equivalent to AtMYB30 encompass: Os03g0378500, Os09g0414300, Os08g0437200, Os11g0558200, and Ob07g0629000 which are homologous transcription factors in Oryza sativa; Sb07021430, Sb02g024640, Sb07g021420, Sb02g040160, Sb05g021820, Sb05g001730, and Sb08g001800 which are homologous transcription factors in Sorghum bicolor; GSVIVP00016337001, GSVIVP00020968001, and GSVIVP00033681001 which are homologous transcription factors in Vitis Vinifera; POPTR_0017s11880g which is a homologous transcription factor in Populus trichocarpa; Glycine max MYB74 which is a homologous transcription factor in Glycine max; and CICLE_v10012152mg which is a homologous transcription factor in Citrus clementina.

In the present invention, the above transcription factors (homologous transcription factors) functionally equivalent to AtMYB30 are usable. This is clear from the fact that, similarly to an AtMYB30 gene, a transformed plant having an improved biomass productivity per unit area under a high-density planting condition is produced with use of a gene encoding Glycine max MYB74 which is a homologous transcription factor in Glycine max.

If plant genome information is disclosed, the homologous transcription factor can be retrieved by search of genome information as an object to be searched, based on base sequences of a gene. A homologous transcription factor retrieved as a candidate transcription factor is a transcription factor which has for example, a sequence identity of 50% or more, preferably 70% or more, more preferably 90% or more, and most preferably 95% or more with respect to an amino acid sequence of an intended transcription factor. Further, the homologous transcription factor retrieved as a transcription factor is a transcription factor which has, for example, a sequence identity of 85% or more, preferably 90% or more, more preferably 95% or more, and most preferably98% or more with respect to an amino acid sequence of a functional domain (for example, MYB domain of MYB protein) of the intended transcription factor. The value of the sequence identity means a value obtained by use of a computer program that implements by default blast algorithm and a database which stores gene sequence information.

The following genes are known as plant-derived PLA₂α genes, in addition to PLA₂α gene (At2g06925) of Arabidopsis thaliana: Os11g0546600, Os03g0261100, and Os03g0708000 of Oryza sativa; Sb05g021000, Sb01g040430, and Sb01g010640 of Sorghum bicolor; GSVIVP00001547001 of Vitis Vinifera; and the like. Each of the above gene products can also be suitably used as the PLA₂α-like protein in the present invention. Further, examples of known orthologues of the BAK1 gene (At4g33430) encompass At2g13790, At2g13800, At1g34210, At1g71830, and the like. Meanwhile, examples of known BAK1 genes derived from plants except for Arabidopsis thaliana encompass: Os04g0457800, and Os08g0174700 of Oryza sativa; Sb07g004750, Sb06g018760, and Sb04g023810 of Sorghum bicolor; GSVIVP00009544001, GSVIVP00001777001, and GSVIVP00019412001 of Vitis Vinifera; Pp135268, and Pp186598 of Physcomitrella patens; Sm268032, Sm444590, and Sm268158 of Selaginella moellendorffii; and the like. Each of these gene products can also be suitably used as the BAK1-like protein in the present invention.

Respective sequences of the above-described genes and of corresponding proteins are shown in a sequence listing. The following shows SEQ ID NOs of the genes and the corresponding proteins.

[Chem. 2]

SEQ ID NO

| | PROTEIN | GENE |
|---|---|---|
| AtMYB30 (At3g28910) | 11 | 12 |
| BAK1 (At4g33430) | 13 | 14 |
| BRI1 (AT4G39400) | 15 | 16 |
| BES1 (AT1G19350) | 17 | 18 |
| MIEL1 (AT5G18650) | 19 | 20 |
| PLA2a (AT2G26560) | 21 | 22 |
| KCS1 (AT1G01120) | 23 | 24 |
| FDH (AT2G26250) | 25 | 26 |
| AtMYB31 (At1g74650) | 27 | 28 |
| AtMYB60 (At1g08810) | 29 | 30 |
| AtMYB94 (At3g47660) | 31 | 32 |
| AtMYB96 (At5g62470) | 33 | 34 |
| Os03g0378500 | 35 | 36 |
| Os09g0414300 | 37 | 38 |
| Os08g0437200 | 39 | 40 |
| Os11g0558200 | 41 | 42 |
| Os07g0629000 | 43 | 44 |
| Sb07g021430 | 45 | 46 |
| Sb02g024640 | 47 | 48 |
| Sb07g021420 | 49 | 50 |
| Sb02g040160 | 51 | 52 |
| Sb05g021820 | 53 | 54 |
| Sb05g001730 | 55 | 56 |
| Sb08g001800 | 57 | 58 |
| GSVIVP00016337001 | 59 | 60 |
| GSVIVP00020968001 | 61 | 62 |
| GSVIVP00033681001 | 63 | 64 |
| POPTR_0017s11880g | 65 | 66 |
| Glycine max MYB74 | 67 | 68 |
| CICLE_v10012152mg | 69 | 70 |
| Os11g0546600 | 71 | 72 |
| Os03g0261100 | 73 | 74 |
| Os03g0708000 | 75 | 76 |
| Sb05g021000 | 77 | 78 |
| Sb01g040430 | 79 | 80 |
| Sb01g010640 | 81 | 82 |
| GSVIVP00001547001 | 83 | 84 |
| At2g13790 | 85 | 86 |
| At2g13800 | 87 | 88 |
| At1g34210 | 89 | 90 |
| At1g71830 | 91 | 92 |
| Os04g0457800 | 93 | 94 |
| Os08g0174700 | 95 | 96 |
| Sb07g004750 | 97 | 98 |
| Sb06g018760 | 99 | 100 |
| Sb04g023810 | 101 | 102 |
| GSVIVP00009544001 | 103 | 104 |
| GSVIVP00001777001 | 105 | 106 |
| GSVIVP00019412001 | 107 | 108 |
| Pp135268 | 109 | 110 |
| Pp186598 | 111 | 112 |
| Sm268032 | 113 | 114 |
| Sm444590 | 115 | 116 |
| Sm268158 | 117 | 118 |

Further, as described above, activation of AtMYB30 improves the hypersensitive response of a plant to infections of pathogenic bacteria (hereinafter, also referred to as disease resistance which results from activation of the MYB30 signaling pathway). Accordingly, the proteins encoded by the MYB30-related genes encompass even mutants of the proteins of AtMYB30, BAK1 and PLA₂α, provided that these mutants each have a function to improve the disease resistance which results from activation of the MYB30 signaling pathway. In one embodiment, if a polypeptide has an amino acid sequence in which one or several amino acids are deleted, substituted, and/or added from/in/to the amino acid sequence represented by SEQ ID NO: 11, 13 or 21 and the polypeptide improves the disease resistance which results from activation of the MYB30 signaling pathway, such a peptide can be suitably used in the present invention.

Note that imparting disease resistance and/or environmental stress resistance to plants does not always lead to an improvement in plant productivity. For example, there is a report on impairment of growth of a plant body in a case where a gene relevant to disease resistance and/or environmental stress resistance is constitutively expressed in the plant body (see, for example, Nakashima et al. (2007) The Plant Journal 51: 617-630). Some technical measure is required so as to prevent such impairment of plant growth. However, such a technical measure requires a different technique for each gene to be used. Therefore, there is no established technique for preventing such impairment of plant growth, and accordingly, such a technique can be neither common technical knowledge nor an indication of a technical level.

The “one or several” as used in terms of a polypeptide (amino acids) is intended to mean the number of amino acids which a person skilled in the art can delete, substitute or add, by a known mutant peptide preparation method such as site-directed mutagenesis, without excessive experimentation. The number is preferably in a range of 1 to 30, more preferably in a range of 20 or less, still more preferably 1, 2, 3, 4, 5, 6, 7, 8, 9 or 10 (i.e., 10 or less), further still more preferably 1, 2, 3, 4 or 5 (i.e., 5 or less). Note that a person skilled in the art can easily understand an extent of the range of the number of amino acids indicated by the term “one or several”, in accordance with the length of an intended polypeptide, and also can prepare “a polypeptide in which one or several amino acids are deleted, substituted, and/or added” without excessive experimentation. Moreover, such a polypeptide is not limited to an artificially-mutated polypeptide, but may be an isolated and purified polypeptide of naturally-occurring polypeptide. Further, a person skilled in the art can confirm without any trial and error whether or not the polypeptide has a desired activation level, by following procedures described in the present specification.

The sequence identity with respect to the intended polypeptide, as used in the present specification is preferably 80% or more, more preferably85% or more, still more preferably 90% or more, further still more preferably 95% or more, and most preferably 99% or more.

It has been well known in the field to which the present invention pertains that several amino acids in an amino sequence of a protein can be easily modified without significantly affecting the structure or function of the protein. Further, it has been also well known that some natural proteins have mutants that do not significantly change the structures or functions of these natural proteins.

Preferable mutants have conservative or nonconservative substitution, deletion, or addition of amino acids. Silent substitution, addition, and deletion are preferred, and conservative substitution is especially preferred. These mutations do not change polypeptide activation level of the present invention.

Typical conservative substitutions encompass: substitution of one of aliphatic amino acids Ala, Val, Leu, and Ile with another amino acid; exchange of hydroxyl residues Ser and Thr; exchange of acidic residues Asp and Glu; substitution between amide residues Asn and Gln; exchange of basic residues Lys and Arg; and substitution between aromatic residues Phe and Tyr.

Further, in the present invention, a polynucleotide that hybridizes, under a stringent condition, with the polynucleotide having the base sequence represented by SEQ ID NO: 12, 14, or 22 can be used, as long as the polynucleotide can encode a polypeptide which improves the disease resistance which results from activation of the MYB30 signaling pathway. Such a polynucleotide encompass, for example, (a) a polynucleotide encoding a polypeptide having an amino acid sequence in which one or several amino acids are deleted, substituted, and/or added from/in/to the amino acid sequence represented by SEQ ID NO: 11, 13, or 21 and (b) a polynucleotide having a base sequence in which one or several bases are deleted, substituted, and/or added from/in/to the base sequence represented by SEQ ID NO: 12, 14, or 22.

The “one or several” as used in terms of a polynucleotide (bases) is preferably in a range of 1 to 100, more preferably in a range of 1 to 50, still more preferably in a range of 1 to 30, further still more preferably in a range of 1 to 15. Note that a person skilled in the art can easily understand an extent of the range of the number of bases indicated by the term “one or several”, in accordance with the length of an intended polynucleotide.

The sequence identity with respect to the intended polynucleotide, as used in the present specification, is preferably 80% or more, more preferably 85% or more, still more preferably 90% or more, further still more preferably 95% or more, and most preferably 97% or more.

In the present invention, the “stringent condition” means that hybridization occurs only when sequences are at least 90%, preferably at least 95%, most preferably at least 97% identical to each other. More specifically, the stringent condition may be, for example, a condition where polynucleotides are incubated in a hybridization solution (50% formamide, 5×SSC (150 mM NaCl, 15 mM trisodium citrate), 50 mM sodium phosphate (pH 7.6), 5× Denhart's solution, 10% dextran sulfate, and 20 μg/ml of sheared denatured salmon sperm DNA) overnight at 42° C., and then the filter is washed with 0.1×SSC at about 65° C.

The hybridization can be carried out by well-known methods such as a method disclosed in Sambrook et al., Molecular Cloning, A Laboratory Manual, 3rd Ed., Cold Spring Harbor Laboratory (2001). Normally, stringency increases (hybridization becomes difficult) at a higher temperature and at a lower salt concentration. At a higher stringency, a more homologous polynucleotide can be obtained.

Sequence identity between amino acid sequences or between base sequences can be determined by use of an algorithm BLAST according to Karlin and Altschul (Karlin S and Altsuchul S F, (1990) Proc. Natl. Acad. Sci. USA, 87: 2264-2268; and (1993) Proc. Natl. Acad Sci. USA, 90: 5873-5877). Programs based on the algorithm BLAST, called BLASTN and BLASTX, have been developed (Altschul SF, et al., (1990) J. Mol. Biol., 215: 403).

The MYB30-related gene for use in the present invention may be derived from genomic DNA or cDNA, and may be chemosynthetic DNA. Further, the MYB30-related gene may be RNA.

A method for obtaining the MYB30-related gene for use in the present invention may be a method according to which a DNA fragment encoding a protein of the MYB30-related gene is isolated and cloned, by use of a well-known technique. For example, the method may include preparing probes that specifically hybridize with part of a base sequence of DNA encoding a protein of MYB30, PLA₂α, or BAK1 of Arabidopsis thaliana and screening a genomic DNA library or a cDNA library with the probes.

Alternatively, the method for obtaining the MYB30-related gene for use in the present invention can be a method using amplification means such as PCR. For example, primers are prepared respectively from sequences on the 5′ side and the 3′ side (or their complementary sequences) of cDNA of the MYB30-related gene of Arabidopsis thaliana. Then, PCR or the like is performed with use of the primers and genomic DNA (or cDNA) as a template, so as to amplify a DNA region between the annealed primers. This makes it possible to obtain a great amount of DNA fragments containing open reading frames of the MYB30-related gene for use in the present invention.

The MYB30-related gene for use in the present invention can be obtained from tissue or cells of an arbitrary plant as a source. Since all plants have an MYB30-related gene, the MYB30-related gene for use in the present invention may be obtained from an intended plant as a source.

[2: Plant Body Suitable for High-Density Planting and Use Thereof]

Plants have been deeply involved with human not only as foods, but as ornaments, industrial materials such as paper and chemicals, and fuels. Further, recently, plants have been spotlighted as biomass energy that will substitute for fossil fuel. However, mechanisms of germination, growth, flowering, and the like of plants have not yet been clarified in many regards. Consequently, cultivation of plants has been mainly based on experiences and intuition, and harvest of the plants has been greatly influenced by natural conditions such as weather. Therefore, clarification of plants' mechanisms of germination, growth, flowering, and the like of plants, and regulating and controlling the mechanisms are very important not only for increasing yields of ornamental plants and food plants such as cereals and vegetables, but also for growing woods in forests and biomass energy.

As shown in Examples described later, it has been confirmed that a transformant in which the MYB30-related gene is introduced causes an increase in biomass quantity per unit area in high-density planting as compared to a parent plant or a wild-type plant. Further, it has also been confirmed in Examples described later that when a plant body has a higher level of MYB30-related gene activity, the plant body is increased in biomass quantity per unit area in high-density planting as compared to a parent plant or a wild-type plant. In other words, the present invention provides (a) a plant body which has an activated MYB30 signaling pathway and which is increased in biomass quantity per unit area in high-density planting, and (b) a method for producing the plant body.

Patent Literature 2 discloses that a plant has an increased biomass quantity per unit area in high-density planting when the plant is (a) a plant having undergone mutation that causes an increase in expression level or activation level of an endogenous γ-glutamylcysteine synthetase (GSH1) of the plant or (b) a transformed plant in which a plant-derived GSH1 gene is introduced. However, the GSH1 gene is not an MYB30-related gene. This is clear from the fact that a GSH1 transformant causes increases in both biomass quantity per unit area in high-density planting and in seed yield, whereas an MYB30 transformant causes a decrease in seed yield.

In one embodiment, the present invention provides a plant body having a higher level of MYB30-related gene activity. The plant body in accordance with the present embodiment can be a plant in which an expression level of an endogenous MYB30-related gene is increased due to artificial mutagenesis or naturally occurring mutation, or a plant in which an endogenous MYB30-related gene is activated due to artificial mutagenesis or naturally occurring mutation. In other words, the method for producing the plant body in accordance with the present embodiment includes the step of inducing artificial mutation of an endogenous MYB30-related gene.

In another embodiment, the present invention provides a transformed plant obtained by transformation with use of an exogenous gene which contains an MYB30-related gene, which transformed plant is increased in biomass quantity per unit area in high-density planting as compared to a parent plant. In other words, the method for producing the plant body in accordance with the present embodiment includes the step of transforming a plant body with use of an exogenous gene which contains an MYB30-related gene.

In the exogenous gene used for transformation of a plant body, a promoter functioning in a plant cell is connected upstream of the MYB30-related gene, while a terminator functioning in a plant cell is connected downstream of the MYB30-related gene. A target plant body can be transformed by introducing such an exogenous gene into the plant body.

Examples of the terminator functioning in a plant cell can be a terminator derived from a nopaline synthetase (NOS) gene, a terminator derived from cauliflower mosaic virus, and the like terminators.

A cauliflower mosaic virus 35S promoter that induces constitutive gene expression is often used as a promoter functioning in a plant cell, but the promoter is not limited to this. Examples of a constitutive promoter other than the cauliflower mosaic virus 35S promoter can be an actin promoter of Oryza sativa, a ubiquitin promoter of Zea mays, and the like. These promoters can also be suitably used in the present invention.

Examples of a promoter other than the constitutive promoter may be chloroplast tissue-specific promoters such as an rbcS promoter and a Cab promoter, inducible promoters such as an HSP70 promoter, and the like, but the promoter is not limited to these. Further, an rbcL promoter and the like promoters can be used as a promoter to be directly inserted into a chloroplast genome, but the promoter is not limited to these provided that the promoter functions in a chloroplast.

A recombinant expression vector as one embodiment of an exogenous gene for use in the present invention is not especially limited provided that the recombinant expression vector can express an MYB30-related gene in a plant cell. Especially, in a case where a method using Agrobacterium is adopted as a method for introducing a vector into a plant body, it is preferable to use a binary vector of a pBI system or the like. Examples of the binary vector encompass: pBIG, pBIN19, pBI101, pBI121, pBI221, pMAT137, and the like.

A target plant body to be transformed in the present invention encompasses a whole plant body, a plant organ (e.g., a leaf, a petal, a stem, a root, a seed), plant tissue (e.g., epidermis, phloem, parenchyma, xylem, bundle, palisade layer, spongy tissue), a cultured plant cell, a variously-altered plant cell (e.g., suspension-cultured cell), a protoplast, a section of a leaf, callus, and the like. The plant body for use in transformation is not especially limited, and a plant in which an MYB30-related gene to be used can be expressed may be selected as appropriate.

In a case where the MYB30-related gene of Arabidopsis thaliana is used, the target plant to be transformed is preferably plants of Brassicaceae closely related to Arabidopsis thaliana, but is not limited to this. It has been reported that intended transformed plants can be produced from various plants by using genes of the various plants or genes derived from other plants (see Franke R et al. (2000) Plant J. 22: 223-234; Yamaguchi and Blumwald (2005) TRENDS in Plant Science 10(12): 615-620). Similarly, transfection of the MYB30-related gene of Arabidopsis thaliana into a plant like the above-described plants allows easy production of a transformed plant suitable for high-density planting, that is, a plant having an improved productivity per unit area under a high-density planting condition.

The present invention is applicable to various plants. This is clear from the fact that when an AtMYB30 gene is transfected into Oryza sativa, in which a homologous transcription factor of the AtMYB30 gene is expressed, it is possible to produce transformed Oryza sativa having an improved biomass productivity per unit area under a high-density planting condition.

Introduction of a recombinant expression vector into a plant cell is carried out by a transformation method well known to a person skilled in the art (for example, an Agrobacterium method, a particle gun method, a polyethylene glycol method, an electroporation method, and the like). In a case where the Agrobacterium method is used, for example, a transformed plant can be obtained by introducing a constructed plant expression vector into appropriate Agrobacterium (for example, Agrobacterium tumefaciens), and then infecting the strain with an aseptically-cultured lamina by a leaf disc method (Hirofumi UCHIMIYA, “Shokubutsu Idenshi Sousa” (Plant Genetic Manipulation Manual), 1990, pp. 27-31, Kodansha Scientific, Tokyo), or the like method.

Further, in a case where the particle gun method is used, a plant body, a plant organ, and plant tissue may be directly used, or alternatively they may be used after they are sectioned to pieces or protoplasts thereof are prepared. A sample so prepared can be processed by use of a gene-introduction device (for example, PDS-1000, manufactured by BIO-RAD). Processing conditions vary depending on the plant or the sample, but are typically as follows: a pressure of approximately 450 to 2000 psi, and a distance of approximately 4 to 12 cm.

Cells or plant tissue into which an intended gene has been introduced is first selected by screening with the use of a drug-resistant marker such as a kanamycin-resistant marker or a hygromycin-resistant marker, and then, the cells or plant tissue thus selected by screening is regenerated into a plant body by a usual method. Regeneration of a plant body from the transformed cell can be carried out by a person skilled in the art by use of a publicly known method depending on the type of the plant cell.

Whether or not an intended gene has been introduced into a plant can be confirmed by a PCR method, a southern hybridization method, a northern hybridization method, or the like method. For example, DNA is prepared from a transformed plant, and primers specific to the introduced DNA are designed, and PCR is performed. After that, amplification products are subjected to agarose gel electrophoresis, polyacrylamide gel electrophoresis, capillary electrophoresis, or the like and then stained with, for example, ethidium bromide so that an intended amplification product is detected, whereby the transformation can be confirmed.

Once the transformed plant body that has incorporated the MYB30-related gene in its genome can be obtained, it is possible to obtain progeny from the plant body by sexual reproduction or asexual reproduction. Further, it is possible to carry out mass production of an intended plant body from a reproductive material (for example, seeds or protoplasts) obtained from the plant body or its progeny or clone.

Even when the plant body in accordance with the present invention is planted at a planting density higher than a planting density that sufficiently increases biomass quantity per unit area, it is possible to further increase the biomass quantity per unit area of the plant body as compared to that of a parent plant/wild-type plant. In other words, the plant body in accordance with the present invention can provide, in high-density planting, biomass quantity that can never be obtained by a parent plant/wild-type plant. However, the planting density at which the plant body in accordance with the present invention is planted is not necessarily limited to a planting density higher than the optimal planting density. The planting density is preferably not less than 30%, more preferably not less than 60%, and still more preferably not less than 100% of the optimal planting density of each variety.

As compared to a wild-type plant or a parent plant, the plant body in accordance with the present invention has an increased biomass quantity in high-density planting. Accordingly, whether or not a certain plant body is the plant body in accordance with the present invention can be found by confirming whether or not the certain plant body is increased in the biomass quantity in high-density planting as compared to the wild-type plant or the parent plant. In other words, the method for producing the plant body in accordance with the present invention may further include the step of confirming whether or not the certain plant body is increased in biomass quantity in high-density planting as compared to a wild-type plant or a parent plant.

Further, in the plant body in accordance with the present invention, the MYB30 signaling pathway is activated, so that disease resistance which results from activation of the MYB30 signaling pathway is improved. Therefore, whether or not a certain plant body is the plant body in accordance with the present invention can be found by confirming whether or not disease resistance which results from activation of the MYB30 signaling pathway is improved, concretely, by confirming whether or not resistance to pathogenic bacteria (for example, Xanthomonas campestris or Pseudomonas syringe) is improved. In other words, the method for producing the plant body in accordance with the present invention may further include the step of confirming whether or not disease resistance which results from activation of the MYB30 signaling pathway is improved.

The plant body (i.e., plant body in accordance with the present invention) obtained in accordance with the above procedures can be cultivated at a planting density higher than that which sufficiently increases biomass quantity per unit area, so that the plant body is increased in resulting biomass quantity as compared to a parent plant (or a plant used for transformation). In other words, the present invention provides a plant biomass production method with use of the above-described plant body.

The production method in accordance with the present invention includes the step of cultivating the plant body in accordance with the present invention under a high-density planting condition. In one embodiment, the plant body can be a plant in which an expression level of an endogenous MYB30-related gene is increased due to artificial mutagenesis or naturally occurring mutation, or a plant in which an endogenous MYB30-related gene is activated due to artificial mutagenesis or naturally occurring mutation. In other words, the production method in accordance with the present embodiment can further include the step of inducing artificial mutation of an endogenous MYB30-related gene.

In another embodiment, the plant body can be a transformed plant obtained by transformation with use of an exogenous gene which contains an MYB30-related gene. The production method in accordance with the present embodiment can further include the step of transforming a plant body with use of an exogenous gene which contains an MYB30-related gene.

In the exogenous gene used in the production method of the present embodiment, preferably, the MYB30-related gene is operably connected to a promoter (inducible promoter) which regulates timing of expression and/or an organ where the MYB30-related gene is expressed. In one aspect, the promoter can initiate expression of the MYB30-related gene immediately prior to a flower bud formation stage of a non-transformed plant. In another aspect, the promoter can cause leaf organ-specific expression of the MYB30-related gene.

The plant body to be transformed is not especially limited provided that the plant body is of a plant which has an endogenous transcription factor functionally equivalent to a gene product of the MYB30-related gene. On the publicly known database released to the public by, for example, the NCBI (National Center for Biotechnology Information), it can be confirmed that such a transcription factor functionally equivalent to the MYB30-related gene is present in a wide range of plants from monocotyledons to dicotyledons. In other words, a monocotyledon or a dicotyledon can be widely used as the plant body to be transformed. Examples of the monocotyledon encompass plants belonging to the following families: Lemnaceae including, for example, the genus Spirodela (Spirodela polyrhiza) and the genus Lemna (Lemna aoukikusa, Lemna trisulca); Orchidaceae including, for example, the genus Cattleya, the genus Cymbidium, the genus Dendrobium, the genus Phalaenopsis, the genus Vanda, the genus Paphiopedilum, and the genus Oncidium; Typhaceae; Sparganiaceae; Potamogetonaceae; Najadaceae; Scheuchzeriaceae; Alismataceae; Hydrocharitaceae; Triuridaceae; Poaceae (e.g., Z. mays such as sweetcorn); Cyperaceae; Palmae; Araceae; Eriocaulaceae; Commelinaceae; Pontederiaceae; Juncaceae; Stemonaceae; Liliaceae; Amaryllidaceae; Dioscoreaceae; Iridaceae; Musaceae; Zingiberaceae; Cannaceae; and Burmanniaceae. Further, the dicotyledon is preferably selected from the group including, for example, plants belonging to the following families: Convolvulaceae including, for example, the genus Ipomoea (Ipomoea nil), the genus Calystegia (Calystegia japonica, Calystegia hederacea, Calystegia soldanella), the genus Ipomoea (Ipomoea pes-caprae, Ipomoea batatas), and the genus Cuscuta (Cuscuta japonica, Cuscuta australis); Caryophyllaceae including the genus Dianthus (Dianthus caryophyllus L., etc.), the genus Stellaria, the genus Minuartia, the genus Cerastium, the genus Sagina, the genus Arenaria, the genus Moehringia, the genus Pseudostellaria, the genus Honckenya, the genus Spergula, the genus Spergularia, the genus Silene, the genus Lychnis, the genus Melandryum, the genus Cucubalus; Casuarinaceae; Saururaceae; Piperaceae; Chloranthaceae; Salicaceae; Myricaceae; Juglandaceae; Betulaceae; Fagaceae; Ulmaceae; Moraceae; Urticaceae; Podostemaceae; Proteaceaes; Schoepfiaceae; Santalaceae; Loranthaceae; Aristolochiaceae; Mitrastemonaceae; Balanophoraceae; Polygonaceae; Chenopodiaceae; Amaranthaceae; Nyctaginaceae; Theligoneae; Phytolaccaceae; Aizoaceae; Portulaceae; Magnoliaceae; Trochodendraceae; Cercidiphyllaceae; Nymphaeaeceae; Ceratophyllaceae; Ranunculaceae; Lardizabalaceae; Berberidaceae; Menispermaceae; Calycanthaceae; Lauraceae; Papaveraceae; Capparaceae; Cruciferae; Droseraceae; Nepenthaceae; Crassulaceae; Saxifragaceae; Pittosporaceae; Hamamelidaceae; Platanaceae; Rosaceae; Leguminosae; Oxalidaceae; Geraniaceae; Linaceae; Zygophyllaceae; Rutaceae; Simaroubaceae; Meliaceae; Polygalaceae; Euphorbiaceae; Callitrichaceae; Buxaceae; Empetraceae; Coriariaceae; Anacardiaceae; Aquifoliaceae; Celastraceae; Staphyleaceae; Icacinaceae; Aceraceae; Hipocastanaceae; Sapindaceae; Sabiaceae; Balseminaceae; Rhamnaceae; Vitaceae; Elaeocarpaceae; Tiliaceae; Malvaceae; Sterculiaceae; Actinidiaceae; Theaceae; Guttiferae; Elatinaceae; Tamaricaceae; Violaceae; Flacourtiaceae; Stachyuraceae; Passifloraceae; Begoniaceae; Cactaceae; Thymelaeaceae; Elaeagnaceae; Lythraceae; Punicaceae; Rhizophoraceae; Alangiaceae; Melastomataceae; Trapaceae; Onagraceae; Haloragaceae; Hippuridaceae; Araliaceae; Umbelliferae; Cornaceae; Diapensiaceae; Clethraceae; Pyrolaceae; Ericaceae; Myrsinaceae); Primulaceae; Plumbaginaceae; Ebenaceae; Symplocaceae; Styracaceae; Oleaceae; Buddlejaceae; Gentianaceae; Apocynaceae; Asclepiadaceae; Polemoniaceae; Boraginaceae; Verbenaceae; Labiatae; Solanaceae (Solanum lycopersicum etc.); Scrophulariaceae; Bignoniaceae; Pedaliaceae; Orobanchaceae; Geseneriaceae; Lentibulariaceae; Acanthaceae; Myoporaceae; Phrymaceae; Plantaginaceae; Rubiaceae; Caprifoliaceae; Adoxaceae; Valerianaceae; Dipsacaceae; Cucurbitaceae; Campanulaceae; Compositae; and the like. The dicotyledon is more preferably a plant selected from the group consisting of plants belonging to the following families: Cruciferae; Solanaceae; Leguminosae; Poaceae; Myrtaceae; Salicaceae; Rutaceae; Cucurbitaceae; Sterculiaceae; Malvaceae; Euphorbiaceae; Rosaceae; Nymphaeaeceae; Labiatae; Gentianaceae; and Vitaceae. Note that the target plants in the present invention can be not only wild-type plants listed above as examples but also mutants or transformants.

The present invention is applicable to plants widely ranging in kinds from monocotyledons to dicotyledons. This is clear from the fact that it is possible to produce transformed Oryza sativa having an improved biomass productivity per unit area under a high-density planting condition, by introducing an AtMYB30 gene derived from Arabidopsis thaliana that is a dicotyledon into Oryza sativa that is a monocotyledon.

Further, in the production method in accordance with the present embodiment, in a case where it is preferred to collect biomass prior to the flower bud formation stage, it is not necessary to use the inducible promoter. In this case, a plant body to be transformed may be the above-described plants.

[3: Tools of Plant Biomass Production and Use Thereof]

The present invention also provides a kit for improving biomass productivity per unit area of a plant under a high-density planting condition. The kit in accordance with the present invention includes an exogenous gene which contains an MYB30-related gene, for improving productivity per unit area of a plant under a high-density planting condition.

In the exogenous gene, the MYB30-related gene can be operably connected to a promoter which regulates timing of protein expression. Further, the MYB30-related gene is preferably a gene encoding a protein selected from the group consisting of AtMYB30, BAK1, and PLA₂α.

The kit in accordance with the present invention can be used for producing a transformed plant having an improved biomass productivity per unit area under a high-density planting condition. In other words, the present invention provides a method for preparing a transformed plant, the method including the step of transforming a plant body with use of the kit. In this case, the kit in accordance with the present invention can further include a reagent for determining the presence or absence of disease resistance which results from activation of the MYB30 signaling pathway. Further, the preparation method in accordance with the present invention may further include the step of selecting an individual which has an improved disease resistance which results from activation of the MYB30 signaling pathway. This step makes it possible to easily find out whether or not the MYB30 signaling pathway is activated in a resulting transformed plant. Consequently, it is possible to easily find out whether the resulting transformed plant has a desired character which causes an improvement in biomass productivity per unit area under a high-density planting condition. Note that the reagent for determining the presence or absence of disease resistance which results from activation of the MYB30 signaling pathway can be, for example, a hydrogen peroxide-specific fluorescent probe, such as 2,7-Dichlorodihydrofluorescein diacetate (DCFH-DA), Hydroxyphenyl Fluorescein, and BES—H₂O₂—Ac, which hydrogen peroxide-specific fluorescent probe detects hydrogen peroxide released in leaves in association with hypersensitive cell death, but the reagent is not limited to the hydrogen peroxide-specific fluorescent probe. Further, when the presence or absence of disease resistance which results from activation of the MYB30 signaling pathway is determined, pathogenic bacteria are preferably used as a pathogen. Such pathogenic bacteria can be, for example, Xanthomonas campestris, Pseudomonas syringe, and the like, but are not limited to these examples. Such pathogenic bacteria can be a reagent for determining the presence or absence of disease resistance which results from activation of the MYB30 signaling pathway.

The kit in accordance with the present invention may include an additional component other than the above substances, such as the exogenous gene which contains an MYB30-related gene and the reagent. The exogenous gene containing an MYB30-related gene, and the additional component may be provided in an appropriate volume and/or in an appropriate form in one container (for example, bottle, plate, tube, or dish), or provided in separate containers, respectively. The kit in accordance with the present invention may further include an instrument, a culture medium, and/or the like for growing a plant. In addition, in order to provide use of the kit for improving biomass productivity per unit area of a plant under a high-density planting condition, the kit in accordance with the present invention preferably includes instruction manuals which describe procedures for use of the kit for improving biomass productivity per unit area of a plant under a high-density planting condition, or instruction manuals which describe procedures for use of the kit for producing a plant which has an improved productivity per unit area under a high-density planting condition. The “instruction manuals” may be written or printed on paper or other medium or alternatively, may be stored in an electronic medium such as a magnetic tape, a computer-readable disk or tape, or a CD-ROM. The kit in accordance with the present invention may be used for forming the above-described composition including the exogenous gene which contains an MYB30-related gene. Further, the kit may separately include substances to be contained in the above-described composition, or include the above-described composition separately from the additional component.

[4: Marker of Plant Body Preferable for High-Density Planting]

As described above, an increase in expression level or activation level of an MYB30-related gene in a plant body serves as an index for finding out that the plant body has an improved productivity per unit area under a high-density planting condition. In other words, the MYB30-related gene serves as a marker which can be used for screening a plant body which has an improved productivity per unit area under a high-density planting condition.

In other words, the present invention provides a method for screening, by using an MYB30-related gene as a marker, a plant body which has an improved productivity per unit area under a high-density planting condition.

In one embodiment, in order to screen a plant body which has an improved productivity per unit area under a high-density planting condition, a screening method in accordance with the present invention includes the steps of: comparing, with a reference value, an expression level of an MYB30-related gene or an expression level of a protein encoded by the MYB30-related gene; and selecting an individual whose expression level of the MYB30-related gene or of the protein encoded by the MYB30-related gene is higher than the reference value. In another embodiment, in order to screen a plant body which has an improved productivity per unit area under a high-density planting condition, a screening method in accordance with the present invention includes the steps of: comparing, with a reference value, an activation level of a protein encoded by an MYB30-related gene; and selecting an individual whose activation level of the protein is higher than the reference value.

The reference value may be an expression level value or an activation level value which has been obtained in advance from a protein encoded by an MYB30-related gene, or an average value of expression level or activation level of a group used for screening.

As described above, an increase in expression level or activation level of an MYB30-related gene of a plant body is considered to be correlated with an improvement in disease resistance which results from activation of the MYB30 signaling pathway. Therefore, it is possible to find out whether a certain plant body is the plant body in accordance with the present invention, by selecting an individual having an improved disease resistance which results from activation of the MYB30 signaling pathway. In other words, the method for producing the plant body in accordance with the present invention may further include the step of confirming whether or not disease resistance which results from activation of the MYB30 signaling pathway is improved.

The plant body in accordance with the present invention has an activated MYB30 signaling pathway, and therefore has an improved disease resistance which results from activation of the MYB30 signaling pathway. Accordingly, it is possible to screen a plant body having an improved productivity per unit area under a high-density planting condition, by confirming whether or not disease resistance which results from activation of the MYB30 signaling pathway is improved. In other words, the screening method in accordance with the present invention may further include the step of selecting an individual having an improved disease resistance which results from activation of the MYB30 signaling pathway.

[5: Additional Use]

As shown in Examples described later, it is possible to screen a gene which causes an improvement in productivity per unit area of a plant under a high-density planting condition, by a procedure including the following steps: (a) first, seeds from a seed library of T-DNA insertion mutant plants are cultivated, so that first generation seeds are obtained; (b) then, the first generation seeds are cultivated, so that second generation seeds are obtained; (c) further, the second generation seeds are cultivated, so that third generation seeds are obtained; (d) a T-DNA insertion site is identified in genomic DNA from the seeds; and (e) a target gene is identified, which target gene has an open reading frame located within 10 kb of the T-DNA insertion site. In this case, the seeds in at least one of the steps (a) to (c) above should be cultivated under a high-density planting condition and seeds should be obtained from a well-grown individual(s) among individuals thus cultivated.

Subsequently, a plant body is transformed with use of an exogenous gene which contains a gene obtained by screening in accordance with the above procedure. This makes it possible to prepare a transformed plant in accordance with the present invention. In preparation of the transformed plant, it is possible to additionally perform selecting an individual having an improved disease resistance which results from activation of the MYB30 signaling pathway.

As described above, the present invention provides a method for screening a gene which allows an improvement in productivity per unit area of a plant under a high-density planting condition, the method including the steps (a) to (e) above, wherein the seeds in at least one of the steps (a) to (c) are cultivated under a high-density planting condition and seeds are obtained from a well-grown individual(s) among individuals thus cultivated.

The gene screening method in accordance with the present invention may further include the step of (f) selecting an individual having an improved disease resistance which results from activation of the MYB30 signaling pathway.

The specific embodiments discussed in the foregoing detailed explanation of the present invention and Examples described as follows serve solely to illustrate the technical details of the present invention, which should not be narrowly interpreted within the limits of such concrete embodiments and examples, but rather may be applied in many variations within the spirit of the present invention, provided such variations do not exceed the scope of the patent claims set forth below.

Further, all the academic literatures and patent literatures cited in the present specification are incorporated in the present specification as references.

### EXAMPLES

The present invention is described as follows in more detail with reference to Examples. However, the present invention is not limited to the following Examples.

### Example 1

[1] Acquisition of MYB30 Gene

First, PCR primers (ATMYB30_F (HindIII) and ATMYB30_R (XbaI)) were designed and synthesized according to sequence information which was provided open to the public by TAIR (http://www.arabidopsis.org/home.html) so that a fragment containing an ORF region of a gene encoding AtMYB30 (AtMYB30 gene: At3g28910) would be amplified. Note that to an end of each of such primers, a restriction enzyme site (HindIII or XbaI) was added. The restriction enzyme site is a site necessary for introducing an expression vector.

[Chem. 3]
ATMYB30_F (HindIII):

(SEQ ID NO: 1)

5′-AAG CTT ATG GTG AGG CCT CCT TGT TGT G-3′
ATMYB30_R (XbaI):

(SEQ ID NO: 2)

5′-TCT AGA CCG GAT ATG AGC GAG CAT TTT TTG GTC-3′

Wild-type Arabidopsis thaliana, ecotype Col-0, was cultivated and harvested young leaves were ground in liquid nitrogen. Then, a DNA preparation kit (DNeasy Plant Mini Kit) manufactured by QIAGEN was used, so that DNA was prepared according to the standard protocol attached to the DNA preparation kit. The DNA thus prepared was used as a template for a PCR reaction which was performed by using enzyme KOD-Plus (manufactured by TOYOBO Co., Ltd.), primers ATMYB30_F (HindIII) and ATMYB30_R (XbaI). Table 1 shows liquid composition for the reaction, while Table 2 shows conditions of the reaction.

TABLE 1

| | | |
|---|---|---|
| Template (Genomic DNA) | 60 | ng |
| 10 × PCR Buffer for KOD-Plus-(Manuractured by TOYOBO) | 5 | μL |
| 2 mM dNTPs (Manuractured by TOYOBO) | 5 | μL |
| 25 mM MgSO₄ | 2 | μL |
| Each of Primers | 20 | pmol |
| KOD-Plus- | 1.0 | unit |
| Total Volume | 50 | μL |

TABLE 2

| | |
|---|---|
| #1 | 94° C. (2 min) |
| #2 | (94° C. (15 sec)/63° C. (30 sec)/68° C. (1 min)) × 25 cycles |

A PCR amplification product was subjected to electrophoresis with use of 2% agarose gel (TAE buffer), and then fragments of the PCR amplification product was stained with ethidium bromide. Thereafter, gel containing an intended fragment was cut and then, the intended DNA fragment was eluted and purified by using QIAquick Gel Extraction Kit (manufactured by QIAGEN). To the DNA fragment thus obtained, adenine was added by using A-Addition Kit (manufactured by QIAGEN). Thereafter, amplified DNA to which adenine was added was ligated into a TA cloning vector, which was followed by transformation of competent cells (DH5α, Nippon Gene) with use of the vector after a ligation reaction. For the above procedures, pGEM-T Easy Vector System (manufactured by Promega Corporation) was used and the transformation was performed according the protocol attached to a corresponding kit. Then, a resulting transformation reaction solution was spread on an LB culture medium plate (containing 50 μg/mL of ampicillin), so that colonies appeared on the culture medium plate. These colonies were subjected to liquid culture in an LB liquid culture medium, so that bacterial cells were obtained. From the bacterial cells, plasmid DNA was prepared by using Plasmid Mini Kit (manufactured by QIAGEN). Thereafter, sequencing of a base sequence and sequence analysis were carried out, and a vector containing an ORF of the AtMYB30 gene was cloned.

[2] Preparation of Plant Expression Vector

A construct was prepared by inserting the fragment containing the ORF of the AtMYB30 gene into a plant expression vector pMAT137 containing a 35S promoter derived from cauliflower mosaic virus.

First, the cloned vector containing the AtMYB30 gene was digested with restriction enzymes HindIII and SacI. Further, pMAT137 was digested with restriction enzymes HindIII and SacI. Digestion products obtained as a result of digestion with the restriction enzymes were subjected to electrophoresis with use of 0.8% agarose gel, and then, an approximately 1.4 kbp fragment containing the ORF of the AtMYB30 gene and a pMAT137 fragment were separately extracted and purified from the gel, by using QIAquick Gel Extraction Kit (manufactured by QIAGEN).

Then, the pMAT137 fragment and the fragment, as a vector, containing the ORF of the AtMYB30 gene were mixed so that a vector: insert ratio will be 1:10. Thereafter, a ligation reaction was performed at 16° C. overnight with TaKaRa Ligation kit ver.2 (manufactured by Takara-Bio Inc.) equal in amount to a resulting vector-and-insert mixture. Then, according to the protocol attached to TaKaRa Ligation kit ver.2, competent cells (DH5α, Nippon Gene) were transformed with use of the vector after the ligation reaction. Subsequently, a resulting transformation reaction solution was spread on an LB agar culture medium (containing 12.5 μg/mL of kanamycin) and culturing was performed overnight, so that colonies appeared in the LB agar culture medium. These colonies were subjected to liquid culture in an LB liquid culture medium, so that bacterial cells were obtained. From the bacterial cells, plasmid DNA was prepared by using Plasmid Mini Kit (manufactured by QIAGEN). Thereafter, sequencing of a base sequence and sequence analysis were carried out, and a plant expression vector containing the ORF of the AtMYB30 gene was obtained.

[3] Gene Transfection into Arabidopsis thaliana by Agrobacterium Method

The plant expression vector prepared above was transfected into Agrobacterium tumefaciens LBA4404 strain by the electroporation method (Plant Molecular Biology Mannal, Second Edition, B. G. Stanton and A. S. Robbert, Kluwer Acdemic Publishers (1994)). Then, the Agrobacterium tumefaciens containing the plant expression vector thus transfected was transduced into the wild-type Arabidopsis thaliana, ecotype Col-0, by the infiltration method described by Clough et al. (Steven J. Clough and Andrew F. Bent (1998) The Plant Journal 16: 735-743).

Thereafter, a plurality of transformed plants was selected with use of a kanamycin-containing medium. The transformed plants thus selected were cultivated and their self-pollination was repeated, so that three kinds of T3 seeds or T4 seeds were obtained, which three kinds were named 18-1, 15-1, and 3-1, respectively.

[4] Confirmation of Gene Expression Level of Transformed Plant

A 26 cm×19.5 cm tray containing soil mixed with vermiculite was divided into 8 partitions, and for each partition, 100 (hundred) T3 seeds obtained above were measured and taken by a seed spoon and sown along one line per partition. Then, the seeds were cultivated for 4 weeks under the conditions of 22° C., 100 μmol/m²/sec, and 16-hour light period/8-hour dark period. Approximately 10 rosette leaves were harvested from plant individuals thus cultivated. Then, real-time PCR was performed to determine an expression level of the AtMYB30 gene in each of transformed plants and a wild-type plant (Col-0). Used as an internal standard was an expression level of 18S ribosomal RNA that is considered to be constitutively expressed in cells.

Then, total RNA was prepared from the rosette leaves harvested, by using RNeasy Plant Mini Kit (manufactured by QIAGEN). PrimeScript (Registered Trademark) RT reagent Kit (Perfect Real Time) (manufactured by Takara-Bio Inc.) was used to prepare cDNA from 1 μg of the total RNA. Table 3 shows liquid composition for the reaction, while Table 4 shows conditions of the reaction.

TABLE 3

| | | |
|---|---|---|
| total RNA | 1 | μg |
| 5 × PrimeScript Buffer | 4 | μL |
| Oligo dT Primer | 50 | pmol |
| Randam 6mers | 100 | pmol |
| PrimeScript RT enzyme Mix I | 1 | μL |
| Total Volume | 20 | μL |

TABLE 4

| | |
|---|---|
| STEP 1 | 37° C. (15 min) |
| STEP 2 | 85° C. (5 sec) |
| STEP 3 | 4° C. |

The real-time PCR was performed in accordance with the following reaction cycles, by using Power SYBR Green PCR Master Mix (manufactured by Applied Biosystems) and 7500 Real Time PCR System (manufactured by Applied Biosystems). Note that cDNA to be used as a template was diluted 5-fold when used for detection of AtMYB30, and diluted 500-fold when used for detection of 18S rRNA. Further, 10-fold serial dilutions at a concentration in a range of 0.0001 ng to 10 ng were prepared, as controls, by using the genome of the wild-type Arabidopsis thaliana Col-0 as a template. Table 5 shows liquid composition for the reaction, while Table 6 shows conditions of the reaction.

TABLE 5

| | |
|---|---|
| Template | 1 μL |
| Forward Primer | 10 pmol |
| Reverse Primer | 10 pmol |
| 2 × Power SYBR Green PCR Master Mix | 12 μL |
| Total Volume | 24 μL |

TABLE 6

| | |
|---|---|
| STEP 1 | 50° C. (2 min) |
| STEP 2 | 95° C. (10 min) |
| STEP 3 | (95° C. (15 sec)/60° C. (1 min)) × 40 cycles |
| STEP 4 | 95° C. (15 sec)/60° C. (1 min) → 95° C. (15 sec)/ |
| | 60° C. (15 sec) |

The following shows respective sequences of primers used for amplification of the AtMYB30 gene and the 18s rRNA.

[Chem. 4]
myb30
At3g28910F:

(SEQ ID NO: 3)

5′-GTG AAA AAC TCG CCG AAG AC-3′
At3g28910R:

(SEQ ID NO: 4)

5′-GCA CAC TCC TTC CCA TCA TC-3′
18S rRNA
At18S F:

(SEQ ID NO: 5)

5′-TCC TAG TAA GCG CGA GTC ATC-3′
At18S R:

(SEQ ID NO: 6)

5′-CGA ACA CTT CAC CGG ATC AT-3′

The expression levels of the AtMYB30 genes were calculated from determination results. Then, the expression levels of the wild type (col-0) and each of the transformed plants (3-1, 15-1, and 18-1) were compared with each other.

[5] Confirmation of Phenotypic Characteristics of Transformed Plants

In 38.44 cm² pots containing soil mixed with vermiculite, the T4 seeds prepared were sown in four sowing patterns. In the four sowing patterns, 1, 3, 8, and 16 seeds of the T4 seeds were sown, respectively, and 35 pots were prepared for each pattern. Then, these seeds were cultivated for 4 weeks under the conditions of 22° C., 100 μmol/m²/sec, and 16-hour light period/8-hour dark period. The 35 pots of each of the four patterns were put in a corresponding tray and managed. In each of the trays, the 35 pots were arranged in 7 lines×5 rows, and 15 pots around the center of a population were used for measurement. In addition to the transformed plants, the wild-type Arabidopsis thaliana (Col-0) was used as a control non-recombinant plant. After the above 4-week cultivation, the fresh weight (biomass quantity) of aerial part of each plant body was weighed by an electronic balance.

[6] Confirmation of Gene Expression Levels of Transformed Plants

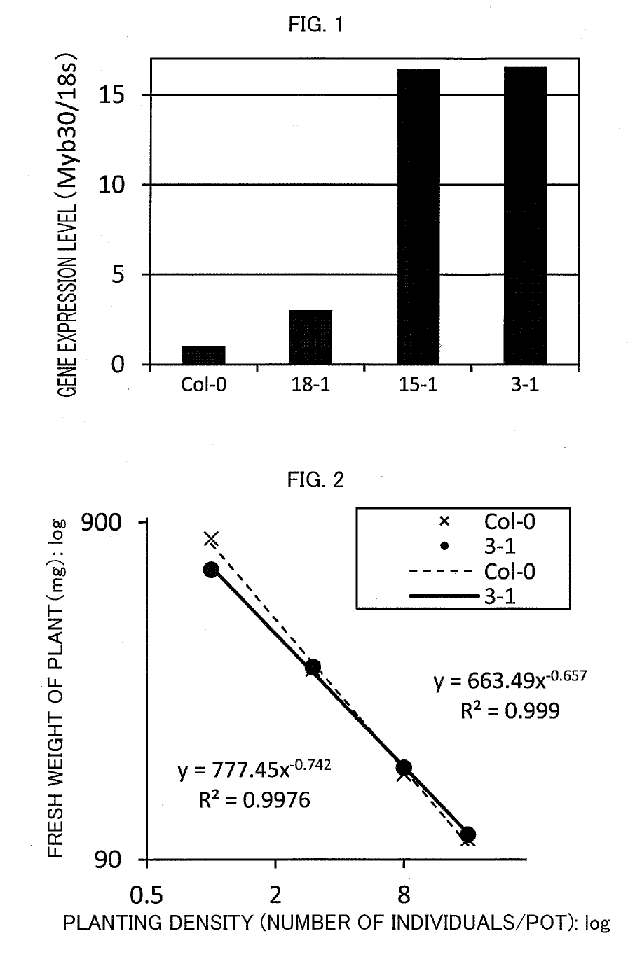

FIG. 1 shows the respective expression levels of the AtMYB30 genes of the transformed plants (18-1, 15-1, and 3-1) four weeks after sowing relative to the expression level of the AtMYB30 gene of the wild type (Col-0) four weeks after sowing. As a result, it was confirmed that more AtMYB30 genes were expressed in the transformed plants than in the wild-type plant. Further, the ascending order of the expression levels were as follows: Col-0<18-1<15-1<3-1.

[7] Phenotypic Characteristics of Transformed Plants

FIG. 2 shows, in a log-log graph, a relationship between the fresh weight of the aerial part of and planting density of each of the wild type (Col-0) and the transformed plant (3-1) into which the fragment containing the ORF of the AtMYB30 gene was introduced. In FIG. 2, dotted line indicates approximate line of the wild-type strain (Col-0), while solid line indicates approximate line of the transformed plant (3-1).

The weight of an individual plant decreases as the planting density increases. The relationship of the planting density and the plant individual is known to follow a rule called “−3/2 power law” and further, the slopes of the approximate lines in the log-log graph is known to be constant according to this rule. However, it was found that the slope of the approximate line of the transformed plant (3-1) in the log-log graph is low. Though the wild-type plant was higher in individual plant weight in low-density planting or optimal density planting than the transformed plant, the transformed plant was higher in individual plant weight under a high-density planting condition than the wild-type plant. This result shows that the transformed plant has a lower degree of decrease in individual plant weight which decrease is associated with an increase in planting density.

When the graph of the planting density and the fresh weight was expressed as Y=bX^{a}, where the planting density was X and the fresh weight was Y, the following mathematical expressions were consequently obtained as mathematical expressions of approximate curves in the graph.

WILD TYPE(Col-0): Y=777.45X^{-0.742}(R²=0.9976)

TRANSFORMED PLANT(18-1): Y=770.30X^{-0.722}(R²=0.9973)

TRANSFORMED PLANT(15-1): Y=706.53X^{-0.678}(R²=0.9948)

TRANSFORMED PLANT(3-1): Y=663.49X^{-0.657}(R²=0.999) [Chem. 5]

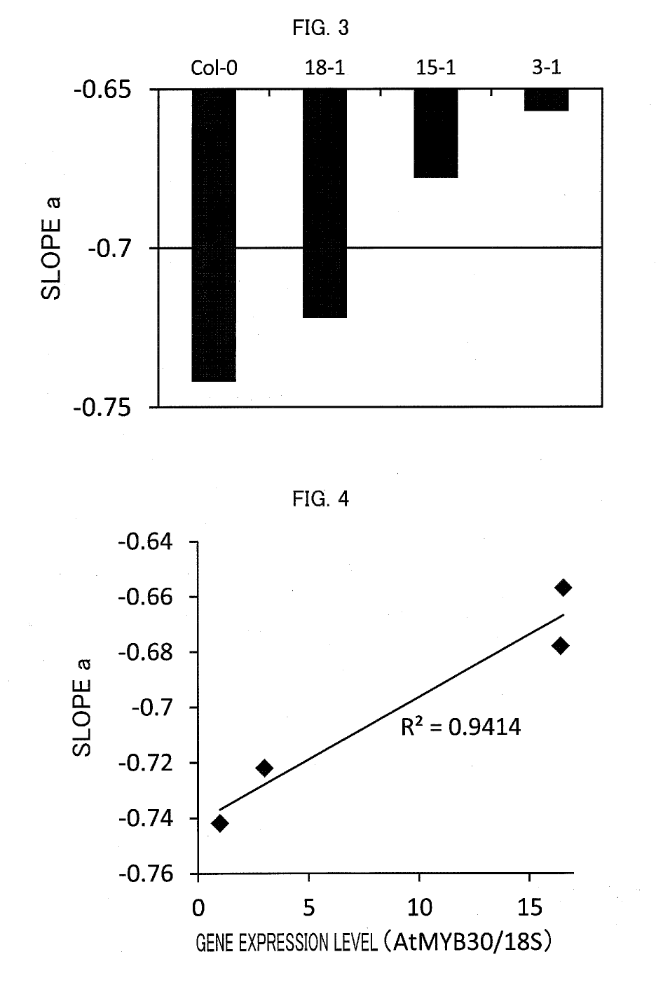

FIG. 3 is a chart for comparing power exponents a indicative of respective slopes in a graph of a wild-type strain and transformed plants. It was found from the chart that the slopes in the descending order are as follows: wild type (Col-0)>18-1>15-1>3-1.

FIG. 4 shows a correlation between (a) the expression levels of the AtMYB30 genes determined by the real-time PCR and (b) the slopes a. It is clear from this graph that the slope of the graph tends to be lower as the expression level of the AtMYB30 gene increases and therefore, an AtMYB30 transformant is an advantageous individual for high-density planting.

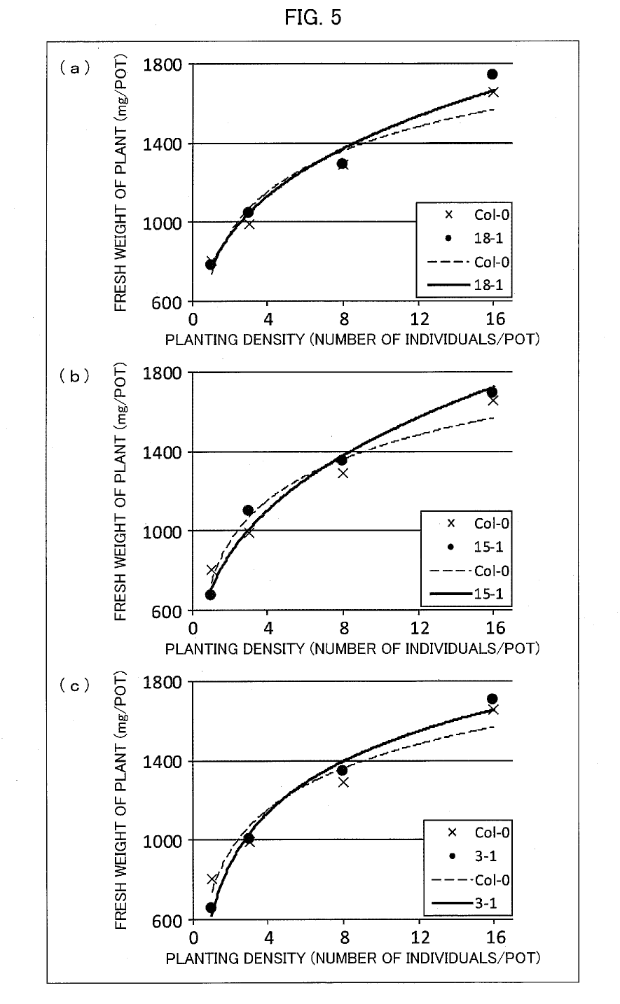

FIG. 5 shows results of comparison of a relationship between the wild type (Col-0) and each of the MYB30 transformed plants ((a) 18-1, (b) 15-1, and (c) 3-1), in regard to biomass yield biomass (fresh weight of aerial part) per pot and planting density. Plotted coordinate marks each indicate a measurement average value, while dotted line and solid line indicate approximate lines. As compared to the wild-type plant, all the transformed plants were higher in biomass quantity per pot under a high-density planting condition. This shows that productivity per unit area can be improved by causing overexpression of the AtMYB30 gene in a plant.

[8] Gene Increasing Plant Biomass Quantity Per Unit Area in High-Density Planting

Seeds of Arabidopsis thaliana mutants (Activation-tag T-DNA lines: Weigel T-DNA lines, 20072 lines in total) were purchased from Nottingham Arabidopsis Stock Centre (NASC). For seeds used in Example 1, see Weigel, D. et al. (2000) Plant Physiol. 122: 1003-1013.

Then, Weigel T-DNA lines were used for selecting strains suitable for high-density planting. In this selection, first, in each 26 cm×19.5 cm tray containing soil mixed with vermiculite, 20 seeds were sown (approximately 2000 seeds in total were sown). For cultivation, a CO₂ chamber (LOW TEMPERATURE O₂/CO₂ INCUBATOR MODEL-9200: WAKENYAKU) was used. In the CO₂ chamber, the seeds were cultured for 4 weeks at a CO₂ concentration of 1% (10,000 ppm), at 22° C., and under illumination at 200 μmol/m²/sec (cycle of 16-hour light period/8-hour dark period). Then, well-grown individuals were selected (first selection) and the individuals thus selected were further cultivated, so that respective seeds of the individuals were obtained.

Furthermore, second selection was performed. In the second selection, a 26 cm×19.5 cm tray containing soil mixed with vermiculite was divided into 8 partitions, and for each partition, 100 plant seeds obtained in the first selection were measured and taken by a seed spoon and sown along one line per partition. Then, these plant seeds were cultured for 4 weeks at a CO₂ concentration of 1% (10,000 ppm), at 22° C., and under illumination at 200 μmol/m²/sec (cycle of 16-hour light period/8-hour dark period), in a CO₂ chamber (LOW TEMPERATURE O₂/CO₂ INCUBATOR MODEL-9200: WAKENYAKU). Then, well-grown individuals were selected. The individuals thus selected were cultivated, so that respective seeds of the individuals were obtained.

Subsequently, young leaves were harvested from the individuals obtained by cultivation of the seeds obtained by selection as above, and the young leaves were ground in liquid nitrogen. Then, the DNA preparation kit (DNeasy Plant Mini Kit) manufactured by QIAGEN was used, so that genomic DNA was prepared according to the standard protocol attached to the DNA preparation kit.

Thereafter, a T-DNA insertion site of the genomic DNA thus prepared was determined by TAIL-PCR. In this determination, first, 3 kinds of specific primers TL1, TL2 and TL3 were designed so as to correspond to a portion in the vicinity of a T-DNA sequence (T-DNA left border) of an activation tagging vector (pSKI015: GenBank accession No. AF187951) which is used in Weigel T-DNA lines.

Each of the above specific primers TL1, TL2 and TL3 was used together with a given primer P1, for performing TAIL-PCR (Kou Shimamoto, and Takuji Sasaki (editing supervisor), New Edition, “Shokubutsu No PCR Jikken Purotokoru” (Protocols of PCR Experiments for Plants), 1997, pp. 83 to 89, Shujunsha Co., Ltd., Tokyo; Liu, Y. G. et al. (1995) The Plant Journal 8: 457-463). Further, the following PCR reaction liquid composition and PCR reaction conditions were also used for performing the TAIL-PCR. As a result of the TAIL-PCR, the genomic DNA adjacent to the T-DNA was amplified.

The following shows respective concrete sequences of the primers TL1, TL2, TL3 and P1.

[Chem. 6]
TL1:

(SEQ ID NO: 7)

5′-TGC TTT CGC CAT TAA ATA GCG ACG G-3′
TL2:

(SEQ ID NO: 8)

5′-CGC TGC GGA CAT CTA CAT TTT TG-3′
TL3:

(SEQ ID NO: 9)

5′-TCC CGG ACA TGA AGC CAT TTA C-3′
P1:

(SEQ ID NO: 10)

5′-NGT CGA SWG ANA WGA A-3′

Note that in the sequence of P1, n represents a, g, c or t (locations: 1 and 11), s represents g or c (location: 7), and w represents a or t (locations: 8 and 13).

Table 7 shows liquid composition for a first PCR reaction, while Table 8 shows conditions of the first PCR reaction.

TABLE 7

| | | |
|---|---|---|
| Template (Genomic DNA) | 10 | ng |
| 10 × PCR Buffer (manufactured by Takara-Bio) | 2 | μL |
| 2.5 mM dNTPs (manufactured by Takara-Bio) | 1.6 | μL |
| First Specific Primer (TL1) | 0.5 | pmol |
| Given Primer (P1) | 100 | pmol |
| TaKaRa Ex Taq (manufactured by Takara-Bio) | 1.0 | unit |
| Total Volume | 20 | μL |

TABLE 8

| | |
|---|---|
| #1 | 94° C. (30 sec)/95° C. (30 sec) |
| #2 | (94° C. (30 sec)/65° C. (30 sec)/72° C. (1 min)) × 5 cycles |
| #3 | 94° C. (30 sec)/25° C. (1 min) → up to 72° C. in 3 min/ |
| | 72° C. (3 min) |
| #4 | 94° C. (15 sec)/65° C. (30 sec)/72° C. (1 min) |
| | 94° C. (15 sec)/68° C. (30 sec)/72° C. (1 min) |
| | (94° C. (15 sec)/44° C. (30 sec)/72° C. (1 min)) × 15 cycles |
| #5 | 72° C. (3 min) |

Table 9 shows liquid composition for a second PCR reaction, while Table 10 shows conditions of the second PCR reaction.

TABLE 9

| | | |
|---|---|---|
| Template (First PCR Product Fiftyfold-Diluted) | 1 | μL |
| 10 × PCR Buffer (manufactured by Takara-Bio) | 2 | μL |
| 2.5 mM dNTPs (manufactured by Takara-Bio) | 1.5 | μL |
| Second Specific Primer (TL2) | 5 | pmol |
| Given Primer (P1) | 100 | pmol |
| TaKaRa Ex Taq (manufactured by Takara-Bio) | 0.8 | unit |
| Total Volume | 20 | μL |

TABLE 10

| | |
|---|---|
| #6 | 94° C. (15 sec)/64° C. (30 sec)/72° C. (1 min) |
| | 94° C. (15 sec)/64° C. (30 sec)/72° C. (1 min) |
| | (94° C. (15 sec)/44° C. (30 sec)/72° C. (1 min)) × 12 cycles |
| #5 | 72° C. (5 min) |

Table 11 shows liquid composition for a third PCR reaction, while Table 12 shows conditions of the third PCR reaction.

TABLE 11

| | | |
|---|---|---|
| Template (Second PCR Product Fiftyfold-Diluted) | 1 | μL |
| 10 × PCR Buffer (manufactured by Takara-Bio) | 5 | μL |
| 2.5 mM dNTPs (manufactured by Takara-Bio) | 0.5 | μL |
| Third Specific Primer (TL3) | 10 | pmol |
| Given Primer (P1) | 100 | pmol |
| TaKaRa Ex Taq (manufactured by Takara-Bio) | 1.5 | unit |
| Total Volume | 50 | μL |

TABLE 12

| | |
|---|---|
| #7 | (94° C. (30 sec)/44° C. (30 sec)/72° C. (1 min)) × 20 cycles |
| #5 | 72° C. (5 min) |

Next, after reaction solutions respectively obtained in the second PCR reaction and the third PCR reaction were subjected to agarose gel electrophoresis, the presence or absence of amplification and reaction specificity were confirmed. Further, the specific primer TL3 and BigDye Terminator Cycle Sequencing Kit Ver.3.1 (manufactured by Applied Biosystems) were used for sequencing of a base sequence of an amplification product in the third PCR reaction. The sequencing of a base sequence was performed by using ABI PRISM 3100 Genetic Analyzer (manufactured by Applied Biosystems). As a result, three pieces (SEQ ID NOs: 12, 14 and 22) of sequence information were obtained from three plant bodies from among selected plant bodies.

The sequence information thus obtained was searched for in BLAST of the Arabidopsis Information Resource (TAIR: http://www.arabidopsis.org/). As a result, it was found that in each of the three pieces of sequence information, an open reading frame (ORF) gene of At3g28910 (which is the third chromosome of Arabidopsis thaliana) was present within 10 kb of the T-DNA insertion site.

Further, several different plant body lines obtained in the above screening were similarly analyzed. As a result, it was found that a BAK1 gene (At4g33430) and a PLA₂α gene (At2g06925) were present within 10 kb of a T-DNA insertion site of each of the plant body lines.

[9] Results

In regard to the AtMYB30 transformant advantageous for high-density planting, it was found that productivity per unit area is improved as an expression level of the AtMYB30 gene increases. This indicates that determination of the expression level of AtMYB30 makes it possible to screen a plant body which is advantageous for high-density planting and which has an improved productivity per unit area. In other words, AtMYB30 can be used as a marker relevant to suitability for high-density planting and to productivity per unit area.

Further, it was confirmed from the result of screening with use of activation tag lines (Activation-tag T-DNA lines) of the Arabidopsis thaliana that a plant body whose AtMYB30 is activated is advantageous for high-density planting. This suggested that PLA₂α exhibits, in the signaling pathway regulated by AtMYB30, a function similar to that of AtMYB30 in terms of high-density planting, which PLA₂α is a molecule (MYB30-related gene) present downstream of BAK1 and AtMYB30 that are molecules capable of positively regulating the function or expression level of AtMYB30.

### Example 2

Many transcription factors having a high sequence identity with an amino acid sequence of AtMYB30 were found by an NCBI protein Blast search, for the purpose of confirmation of effects of orthologues of an AtMYB30 gene. Among the transcription factors thus found, a GmMYB74 gene derived from Glycine max, which is a major crop of Leguminosae family plants, was selected as a homologous transcription factor of the AtMYB30 gene, and effects of this homologous transcription factor was confirmed. Note that amino acid sequences of GmMYB74 and AtMYB30 show 53% sequence identity with each other.

Both the AtMYB30 gene and the GmMYB74 gene are transcription factors each of which has an MYB domain (R2R3 type). The amino acid sequence (SEQ ID NO: 123) of the MYB domain of AtMYB30 and the amino acid sequence (SEQ ID NO: 124) of the MYB domain of GmMYB74 show 92.3% sequence identity with each other. Accordingly, the amino acid sequences of the MYB domains of AtMYB30 and GmMYB74 have an extremely high sequence identity with each other.

A gene artificial synthesis service provided by GenScript was utilized for artificial synthesis of a sequence (SEQ ID NO: 119) which contains a full-length gene (GmMYB74 gene; SEQ ID NO: 68) encoding GmMYB74. Though Example 1 used a pMAT vector, use of the pMAT vector was not suitable for sequence analysis of an introduced gene because a vector size became too large. Accordingly, Example 2 used a plant expression vector containing a cauliflower mosaic virus 35S promoter, that is, a pGreen II vector (John Innes Center, England). Into this pGreen II vector, a fragment (SEQ ID NO: 120) was inserted. This fragment was obtained by end-blunting of a NotI site (start codon side) and an Hpal site (stop codon side) which were added in the above gene synthesis. The pGreen II vector is a general vector which is known to be suitably usable for transformation of plants such as plants of Brassicaceae, wheat and barley. T4 DNA Polymerase (Takara-Bio) was used for end-blunting, while Rapid DNA Dophos & Ligation kit (Roche) was used for an intended ligation reaction. After the ligation reaction, the vector was used for transformation of competent cells (DH5α, Nippon Gene). The competent cells thus transformed was amplified in an LB agar culture medium (containing 12.5 μg/mL of kanamycin), so that bacterial cells were obtained. Thereafter, plasmid DNA was prepared from the bacterial cells by using QIAprep Spin Miniprep Kit (manufactured by QIAGEN), so that a plant expression vector containing an ORF (SEQ ID NO: 68) of the GmMYB74 gene was obtained. Further, the sequence of an inserted gene in the plant expression vector thus obtained was confirmed.

The plant expression vector containing the GmMYB74 gene was transfected as in Example 1 into Agrobacterium (GV3101 strain), together with pSoup as a helper plasmid. Then, a resulting plant expression vector was transfected into the wild type Arabidopsis thaliana, ecotype Col-0, as in Example 1.

Screening with hygromycin and self-pollination were repeated to give T3 seeds of a strain (#3-2 strain) which expresses the GmMYB74 gene at a high level. Further, it was confirmed that the GmMYB74 gene was homologously inserted into the T3 seeds.

In 38.44 cm² pots containing soil mixed with vermiculite, the #3-2 strain seeds were sown in four sowing patterns. In the four sowing patterns, 1, 3, 8, and 16 seeds of the T4 seeds were sown, respectively, and 25 pots were prepared for each pattern. Then, these seeds were cultivated for 4 weeks under the conditions of 22° C., 100 μmol/m²/sec, and 16-hour light period/8-hour dark period. The 25 pots of each of the four patterns were put in a corresponding tray and managed. In each of the trays, the 25 pots were arranged in 5 lines×5 rows, and 6 to 9 pots around the center of a population were used for measurement. In addition to the transformed plants, the wild-type Arabidopsis thaliana (Col-0) was used as a control non-recombinant plant. After the above 4-week cultivation, the fresh weight (biomass quantity) of aerial part of each plant body was weighed by an electronic balance.

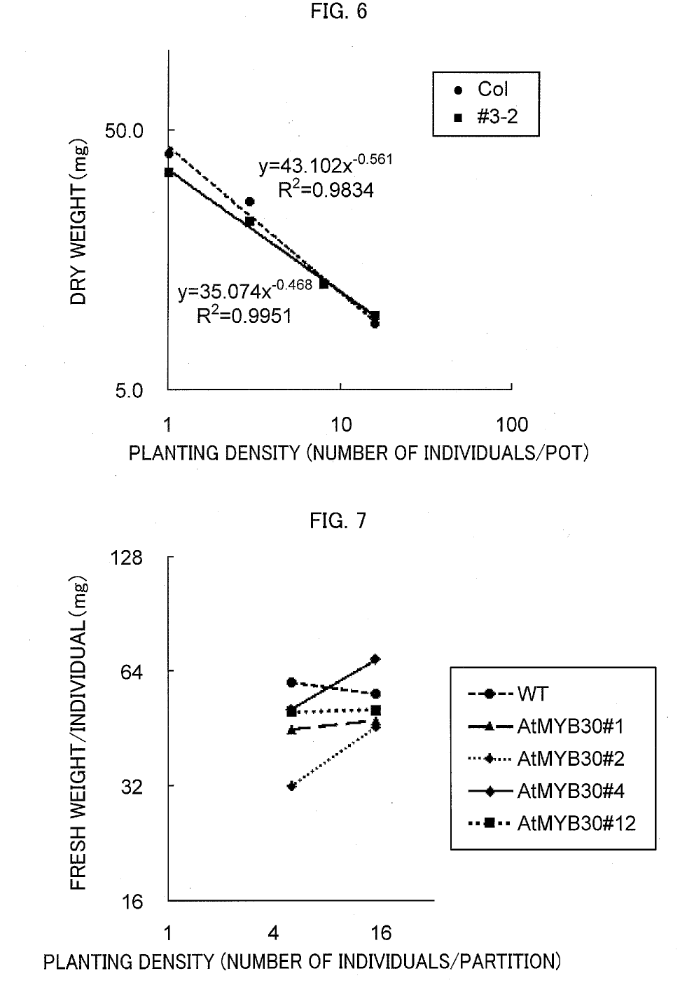

FIG. 6 shows, in a log-log graph, a relationship between dry weight of aerial part of and planting density of each of the wild type (Col-0) and the GmMYB74 transformed plant (#3-2 strain). In FIG. 6, dotted line indicates approximate line of the wild-type strain (Col-0), while solid line indicates approximate line of the transformed plant (#3-2 strain).

As described above, the weight of an individual plant decreases as the planting density increases. The relationship of the planting density and the plant individual is known to follow a rule called “−3/2 power law” and further, the slopes of the approximate lines in the log-log graph is known to be constant according to this rule. However, as in Example 1, it was found that the slope of the approximate line of the transformed plant (#3-2 strain) in the log-log graph is low. Though the wild-type plant was higher in individual plant weight in low-density planting or optimal density planting than the transformed plant, the transformed plant was higher in individual plant weight under a high-density planting condition than the wild-type plant.

These results show that the gene encoding Glycine max MYB74, which is an AtMYB30 homologous transcription factor in Glycine max, reduces, in the similar manner as the AtMYB30 gene, a degree of decrease in individual plant weight, which decrease is associated with an increase in planting density. In other words, the AtMYB30 homologous transcription factor is usable for the present invention.

### Example 3

The AtMYB30 gene obtained in Example 1 was inserted into a pGreen II vector for plant expression. For ligation with the pGreen II vector, a SalI site and a NotI site were added to respective terminuses of the AtMYB30 gene by using primers SalI-AtMYB30_f and NotI-AtMYB30_r.

The following shows respective concrete sequences of the primers SalI-AtMYB30_f and NotI-AtMYB30_r.

[Chem. 7]
SalI-AtMYB30_f:

(SEQ ID NO 121)

5′-ATT AGT CGA CAT GGT GAG GCC TCC TTG-3′
NotI-AtMYB30_r:

(SEQ ID NO 122)

5′-TTA TGC GGC CGC TCA GAA GAA ATT AGT GTT-3′

PCR products, which are obtained by using the above primers, and pGreen II were processed with restriction enzymes (SalI, and NotI), and digestion products obtained by digestion with these restriction enzymes each were subjected to agarose gel electrophoresis. Then, a fragment containing an ORF of the AtMYB30 gene and a fragment of pGreenII were each purified from a resulting gel by using QIAquick Gel Extraction Kit (manufactured by QIAGEN). Thereafter, the fragment containing the ORF of the AtMYB30 gene and the fragment of pGreenII were mixed with each other. Further, a litigation reaction of a predetermined volume was performed at 16° C. for not less than 30 minutes, by using Rapid NA Dophos & Ligation kit (Roche). By using a resulting vector after the ligation reaction, competent cells (DH5α, Nippon Gene) were transformed according to the protocol attached to the Rapid NA Dophos & Ligation kit. Next, a resulting transformation reaction solution was spread on an LB agar culture medium (containing 12.5 μg/mL of kanamycin) and cultured overnight. Then, colonies having appeared on the LB culture medium were subjected to liquid culture in an LB liquid culture medium, so that bacterial cells were obtained. From the bacterial cells, plasmid DNA was prepared by using QIAprep Spin Miniprep Kit (manufactured by QIAGEN), so that a plant expression vector containing the ORF of the AtMYB30 gene was obtained. Further, the sequence of this vector was confirmed.

The plant expression vector thus obtained was used to transform wild-type Oryza sativa (Nipponbare) callus. A plurality of transformed plants was selected with use of a hygromycin-containing culture medium. Then, transformed Oryza sativa (TO) obtained as a result of redifferentiation was cultivated, so that T1 seeds were obtained.

Four pots (9 cm in diameter) were each divided into 4 partitions. Then, 5 seeds or 15 seeds of the T1 seeds were sown in corresponding partitions. Then, the seeds thus sown were cultivated for 2 weeks under the conditions of 25° C., 200 μmol/m²/sec, and 14-hour light period/10-hour dark period. The wile-type Oryza sativa (Nipponbare) was used as a non-transformed plant for control partitions. After 4-seek cultivation, the fresh weight (biomass quantity) of aerial part of each plant body was weighed by an electronic balance.

FIG. 7 shows results of comparison between the wild-type Oryza sativa and the transformed Oryza sativa, in regard to a relationship between yield of biomass (fresh weight of aerial part) per pot and planting density.

In the case of the wild-type plant (WT), a fresh weight per individual was smaller in the partition where 15 seeds had been sown than in the partition where 5 seeds had been sown. In other words, it is clear that in the partition where 15 seeds had been sown, competition of growth occurs. Meanwhile, in the case of the transformed Oryza sativa (AtMYB30#1, AtMYB30#2, AtMYB30#4, and AtMYB30#12) in which an expression level of AtMYB30 was high, the fresh weight per individual was larger in the partition where 15 seeds had been sown than in the partition where 5 seeds had been sown. This means that, even under the condition where 15 seeds had been sown in one partition under which condition competition of growth occurred in the case of the wild-type plant (WT), the fresh weight per individual increased in the case of the transformed Oryza sativa in which an expression level of AtMYB30 was high. This indicates that no competition of growth occurred in the case of the transformed Oryza sativa and that the transformed Oryza sativa in which an expression level of AtMYB30 was high can more advantageously grow under a high-density planting condition than the wild-type plant.

As described above, introduction of the AtMYB30 gene into Oryza sativa which expresses an AtMYB30 homologous transcription factor makes it possible to produce a transformed Oryza sativa having higher biomass productivity per unit area under a high-density planting condition. Further, the function of a dicotyledon-derived gene is found in monocotyledons. These support that various types of plants can be used in the present invention.

### INDUSTRIAL APPLICABILITY

The present invention makes it possible to increase plant biomass yield. Therefore, the present invention is applicable not only to agriculture and forestry but also to a wide range of industries such as food industry and energy industry.

### SEQUENCE LISTING

TJ15186_sequence.txt

1. A method for producing plant biomass, comprising the step of cultivating a plant body in which an MYB30 signaling pathway is activated, the plant body being cultivated under a high-density planting condition.

2. The method as set forth in claim 1, wherein the plant body is a transformed plant obtained by transformation with an exogenous gene which contains an MYB30-related gene.

3. The method as set forth in claim 2, wherein in the exogenous gene, the MYB30-related gene is operably connected to an inducible promoter which regulates expression timing.

4. The method as set forth in claim 2, wherein the MYB30-related gene is a gene encoding a protein selected from the group consisting of AtMYB30, BAK1 and PLA₂α.

5. The method as set forth in claim 1, further comprising the step of collecting biomass after cultivation of the plant body.

6. A kit for improving biomass productivity per unit area of a plant under a high-density planting condition, the kit comprising an exogenous gene which contains an MYB30-related gene.

7. The kit as set forth in claim 6, further comprising a reagent for determining the presence or absence of disease resistance which results from activation of an MYB30 signaling pathway.

8. (canceled)

9. (canceled)

10. (canceled)

11. A method for screening a plant body having an improved productivity per unit area under a high-density planting condition, the method comprising the steps of:
comparing, with a reference value, an expression level of an MYB30-related gene or an expression level of a protein encoded by the MYB30-related gene; and
selecting an individual whose expression level of the MYB30-related gene or of the protein encoded by the MYB30-related gene is higher than the reference value.

12. A method for screening a plant body having an improved productivity per unit area under a high-density planting condition, the method comprising the steps of:
comparing, with a reference value, an activation level of a protein encoded by an MYB30-related gene; and
selecting an individual whose activation level of the protein is higher than the reference value.

13. The method as set forth in claim 11, further comprising the step of selecting an individual having an improved disease resistance which results from activation of an MYB30 signaling pathway.

14. The method as set forth in claim 12, further comprising the step of selecting an individual having an improved disease resistance which results from activation of an MYB30 signaling pathway.
